(19) United States
(12) Patent Application Publication
(10) Pub. No.: US 2026/0324277 A1
(43) Pub. Date: Oct. 15, 2026

(54) COMPOUND AND ORGANIC LIGHT-EMITTING DEVICE COMPRISING SAME

(71) Applicant: LG CHEM, LTD, Seoul (KR)

(72) Inventors: Sujeong GEUM, Daejeon (KR); Jiye JEON, Daejeon (KR); Woo Jin CHO, Daejeon (KR); Ji Young CHOI, Daejeon (KR); Jae Seung HA, Daejeon (KR); Jongsoo SONG, Daejeon (KR); Woochul LEE, Daejeon (KR)

(21) Appl. No.: 19/481,583

(22) PCT Filed: Nov. 6, 2024
(86) PCT No.: PCT/KR2024/017424

§ 371 Date: Nov. 5, 2025

(30) Foreign Application Priority Data
Nov. 6, 2023 (KR) 10-2023-0151979

(51) Int. Cl. H10K 85/60; C07B 59/00; C07D 307/77; C07D 407/10; C09K 11/02; C09K 11/06; H10K 50/12; H10K 101/00

(52) U.S. Cl. CPC H10K 85/622; C07B 59/002; C07D 307/77; C07D 407/10; C09K 11/02; C09K 11/06; H10K 85/626; H10K 85/6574; H10K 85/658; C09K 2211/1007; C09K 2211/1018; H10K 50/12; H10K 2101/90

(57) ABSTRACT

A compound of the following Chemical Formula 1, and an organic light-emitting device containing the compound in at least one organic material layer between the anode and the cathode:

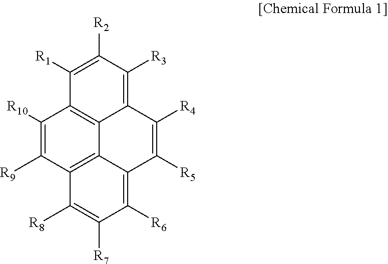

where one to four of R4, R5, R9 and R10 is independently connected to L of Chemical Formula 2:

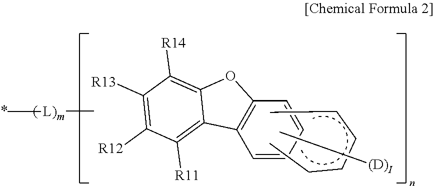

and L is connected to any one of R11 to R14 of Chemical Formula 2, the remainder of R4. R5, R9 and R10 not connected to L, RI to R3, R6 to R8, and the remainder of R11 to R14 not connected to L, are identical or different from each other, and are independently hydrogen, deuterium, a substituted or unsubstituted tetrahydronaphthalenyl group, a substituted or unsubstituted silyl group, or a substituted or unsubstituted aryl group. * indicates the position of bonding, and the other substituents are as described in the specification.

This application is a National Stage Application of International Application No. PCT/KR2024/017424 filed on Nov. 6, 2024, which claims the benefit of Korean Patent Application No. 10-2023-0151979 filed on Nov. 6, 2023 in the Korean Intellectual Property Office, the entire contents of which are herein incorporated by reference.

### TECHNICAL FIELD

The present disclosure relates to compounds and organic light-emitting devices containing them.

### BACKGROUND ART

Generally, the phenomenon of organic light emission refers to a process wherein electrical energy is converted to light energy using an organic material. An organic light-emitting device that utilize the organic light emission phenomenon typically has a structure that includes an anode, a cathode, and an organic layer therebetween. Here, the organic layer is often composed of a multilayer structure made of different materials to enhance the efficiency and stability of the organic light-emitting device. For example, it can consist of a hole injection layer, a hole transport layer, a light-emitting layer, an electron transport layer, an electron injection layer, etc. In the structure of such an organic light-emitting device, when a voltage is applied between the two electrodes, holes are injected at the anode and electrons are injected at the cathode into the organic layer. Excitons are formed when the injected holes and electrons meet with each other. Light is emitted when these excitons return to the ground state.

Development of new materials for the organic light-emitting devices continues to be required.

### BRIEF DESCRIPTION

### Technical Problem

The present disclosure provides compounds and organic light-emitting devices containing them.

### Technical Solution

An exemplary embodiment of the present disclosure provides a compound of the following Chemical Formula 1.

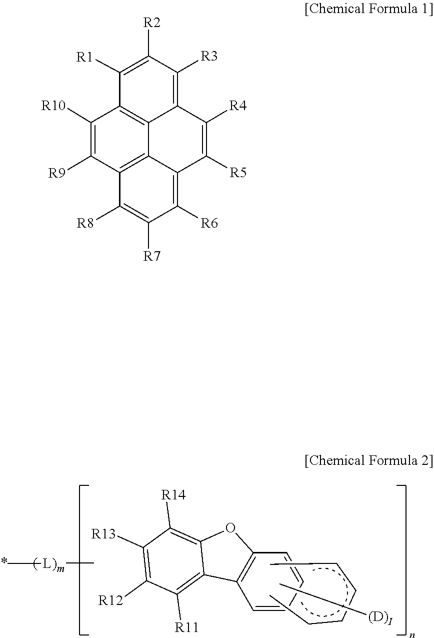

In Chemical Formulas 1 and 2,
one to four of R4, R5, R9 and R10 is independently connected to L of Chemical Formula 2, and L is connected to any one of R11 to R14 of Chemical Formula 2,
the remainder of R4, R5, R9 and R10 not connected to L, R1 to R3, R6 to R8, and the remainder of R11 to R14 not connected to L, which are identical or different, are independently hydrogen, deuterium, a substituted or unsubstituted tetrahydronaphthalenyl group, a substituted or unsubstituted silyl group, or a substituted or unsubstituted aryl group,
L is a single bond, a substituted or unsubstituted arylene group, or a substituted or unsubstituted divalent heterocyclic group,
n is an integer from 1 to 4, and the structures within the parentheses are identical or different if n is 2 or greater,
m is an integer from 1 to 4, and two or more Ls are identical or different if m is 2 or greater,
1 is the number of deuterium, which is an integer from 0 to 6, and
*indicates the position of bonding.

In addition, an exemplary embodiment of the present disclosure provides an organic light-emitting device including an anode; a cathode; and one or more organic layer provided between the anode and the cathode, wherein the one or more of the organic layer contains the compound of Chemical Formula 1.

### Advantageous Effects

The compounds described in the present disclosure can be used as materials for an organic layer of an organic light-emitting device.

Since the compounds according to at least one exemplary embodiment of the present disclosure have a higher triplet energy than conventional anthracene-based compounds, the triplet quenching of compounds (dopants or additional hosts) used together can be reduced and, therefore, the efficiency and lifetime characteristics of organic light-emitting devices can be improved. Particularly, in the case of 2-EML devices used together with anthracene-based compounds, the mechanisms of exciton formation and triplet-triplet fusion greatly contribute to the improved lifespan of the devices.

### BRIEF DESCRIPTION OF THE DRAWINGS

FIG. 1 shows the structure of an exemplary organic light-emitting device in which a substrate 1, an anode 2, an organic layer 60, and a cathode 10 are stacked sequentially. In such a structure, a compound of the present disclosure can be contained in an organic layer 60.

FIG. 2 shows the structure of an exemplary organic light-emitting device in which a substrate 1, an anode 2, a light-emitting layer 6, and a cathode 10 are stacked sequentially. In such a structure, a compound of the present disclosure can be contained in the light-emitting layer 6.

FIG. 3 shows an exemplary organic light-emitting device in which a substrate 1, an anode 2, a hole injection layer 3, a hole transport layer 4, an electron blocking (hole control) layer 5, a light-emitting layer 6, a hole blocking (electron control) layer 7, an electron transport layer 8, an electron injection layer 9, and a cathode 10 are stacked sequentially.

FIG. 4 shows an exemplary organic light-emitting device in which a substrate 1, an anode 2, a hole injection and transport layer 34, a light-emitting layer 6, an electron transport layer 8, an electron injection layer 9, and a cathode 10 are stacked sequentially.

FIG. 5 shows an exemplary organic light-emitting device in which a substrate 1, an anode 2, a hole injection layer 3, a hole transport layer 4, an electron blocking (hole control) layer 5, a light-emitting layer 6, a hole blocking (electron control) layer 7, an electron transport layer 8, and a cathode 10 are stacked sequentially.

FIG. 6 shows an exemplary organic light-emitting device in which a substrate 1, an anode 2, a hole injection layer 3, a hole transport layer 4, an electron blocking (hole control) layer 5, a first light-emitting layer 6-1, a second light-emitting layer 6-2, a hole blocking (electron control) layer 7, an electron transport layer 8, and a cathode 10 are stacked sequentially.

FIG. 7 shows an exemplary organic light-emitting device in which a substrate 1, an anode 2, a hole injection layer 3, a hole transport layer 4, an electron blocking (hole control) layer 5, a first light-emitting layer 6-1, a second light-emitting layer 6-2, a third light-emitting layer 6-3, a hole blocking (electron control) layer 7, an electron transport layer 8, and a cathode 10 are stacked sequentially.

### DESCRIPTION OF MAIN ELEMENTS

1: substrate
2: anode
3: hole injection layer
4: hole transport layer
34: hole injection and transport layer
5: hole control layer
6: light-emitting layer
6-1: first light-emitting layer
6-2: second light-emitting layer
6-3: third light-emitting layer
7: electron control layer
8: electron transport layer
9: electron injection layer
10: cathode
60: organic layer

### DETAILED DESCRIPTION

Hereinafter, the present disclosure is described in more detail.

In the present disclosure, when a certain part is said to “include” a certain component, it means that, unless otherwise stated, it does not exclude other components but may include additional components.

In the present disclosure, when a member is said to be located “on” another member, it includes not only the case where the member is in contact with the another member but also the case where a still another member exists between the two members.

In the present disclosure,

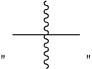

indicates a position that is bonded to a chemical formula or a compound.

Examples of the substituents of the present disclosure are described below, but are not limited thereto.

The term “substitution” refers to a process whereby a hydrogen atom bonded to the carbon atom of a compound is replaced by another substituent. The position of substitution is not limited as long as it is the position where the hydrogen atom is replaced. In cases where there are two or more substitutions, the two or more substituents can be identical or different.

In the present disclosure, the term “substituted or unsubstituted” refers to being substituted with one or more substituent selected from a group consisting of deuterium; a halogen group; a nitrile group (—CN); a nitro group; a hydroxyl group; an alkyl group; a cycloalkyl group; an alkoxy group; a phosphine oxide group; an aryloxy group; an alkylthioxy group; an arylthioxy group; an alkylsulfoxy group; an arylsulfoxy group; an alkenyl group; a silyl group; a boron group; an amine group; an aryl group; or a heterocyclic group, being substituted with a substituent formed as two or more the substituents described above are connected, or having no substituent at all. For example, “a substituent with two or more substituents connected” can be a biphenyl group. That is, the biphenyl group can be an aryl group, or may be interpreted as a substituent with two phenyl groups connected.

In the present disclosure, the term “substituted or unsubstituted” refers to being substituted with one or more substituent selected from a group consisting of deuterium; a halogen group; a nitrile group; a nitro group; a hydroxyl group; an amino group; an alkoxy group; an aryloxy group; an alkyl group; a cycloalkyl group; an aryl group; and a heterocyclic group, being substituted with a substituent formed as two or more the substituents described above are connected, or having no substituent at all.

In the present disclosure, the term “substituted or unsubstituted” refers to being substituted with one or more substituent selected from a group consisting of deuterium; a halogen group; a nitrile group; an alkyl group; an aryl group; and a heterocyclic group, being substituted with a substituent formed as two or more the substituents described above are connected, or having no substituent at all.

The examples of the above substitutes are described below, but are not limited thereto.

In the present disclosure, the examples of the halogen group include fluorine (—F), chlorine (—Cl), bromine (—Br), or iodine (—I).

In the present disclosure, the silyl group can be represented by Chemical Formula —SiYₐY_{b}Y_{c}, wherein each of Yar Y_{b} and Y_{c} may be hydrogen; a substituted or unsubstituted alkyl group; or: a substituted or unsubstituted aryl group. Specifically, the silyl group ma y be trimethylsilyl, triethylsilyl, t-butyldimethylsilyl, vinyldimethylsilyl, propyldimethylsilyl, triphenylsilyl, diphenylsilyl, phenylsilyl, etc., although not being limited thereto.

In the present disclosure, the alkyl group may be linear or branched, and the number of carbon atoms may be specifically 1 to 60, although not being specially limited thereto. According to an exemplary embodiment, the number of carbon atoms in the alkyl group is 1 to 30. According to another exemplary embodiment, the number of carbon atoms in the alkyl group is 1 to 20. According to another exemplary embodiment, the number of carbon atoms in the alkyl group is 1 to 10. Specific examples of alkyl group include a methyl group, an ethyl group, a propyl group, a n-propyl group, an isopropyl group, a butyl group, a n-butyl group, an isobutyl group, a tert-butyl group, a pentyl group, a n-pentyl group, a hexyl group, a n-hexyl group, a heptyl group, a n-heptyl group, an octyl group, a n-octyl group, etc., although not being limited thereto.

In the present disclosure, the foregoing description of the alkyl group is applicable to the arylalkyl group, except that it is substituted with an aryl group.

In the present disclosure, the alkoxy group may be linear, branched or cyclic. The number of carbon atoms in the alkoxy group may be specifically 1 to 20, although not being specially limited thereto. Specific examples may include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, etc. although not being limited thereto.

The alkyl groups, alkoxy groups, and other substituents containing alkyl moieties described in the present disclosure encompass both linear and branched forms.

An alkoxy group is expressed as —OR, wherein R is an alkyl group. Meanwhile, in the aryloxy group, R is an aryl group.

In the present disclosure, the foregoing description of the alkoxy and aryloxy groups is applicable to the alkylthioxy and arylthioxy group, respectively, except that the O in the alkoxy and the O in aryloxy groups are S.

In the present disclosure, the alkenyl group may be linear or branched, and the number of carbon atoms is specifically 2 to 40, although not being specially limited thereto. According to an exemplary embodiment, the number of carbon atoms in the alkenyl group is 2 to 20. According to another exemplary embodiment, the number of carbon atoms in the alkenyl group is 2 to 10. According to another exemplary embodiment, the number of carbon atoms in the alkenyl group is 2 to 6. Specific examples include a vinyl group, a 1-propenyl group, an isopropenyl group, a 1-butenyl group, a 2-butenyl group, a 3-butenyl group, a 1-pentenyl group, a 2-penentyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group, etc., although not being limited thereto.

In the present disclosure, the alkynyl group is a substituent that contains a triple bond between carbon atoms. It can be linear or branched, and the number of carbon atoms is specifically 2 to 40, although not being specially limited thereto. According to an exemplary embodiment, the number of carbon atoms in the alkynyl group is 2 to 20. According to another exemplary embodiment, the number of carbon atoms in the alkynyl group is 2 to 10.

In the present disclosure, the number of carbon atoms in the cycloalkyl group is specifically 3 to 60, although not being specially limited thereto. In an exemplary embodiment, the carbon number of the cycloalkyl group is 3 to 30. According to another exemplary embodiment, the number of carbon atoms in the cycloalkyl group is 3 to 20. According to another exemplary embodiment, the number of carbon atoms in the cycloalkyl group is 3 to 6. Specific examples include a cyclopropyl group, a cyclobutyl group, a cyclopentyl group, a cyclohexyl group, a cycloheptyl group, a cyclooctyl group, etc., although not being limited thereto.

In the present disclosure, the amine group refers to —NH₂. The amine group can be substituted with an alkyl group, an aryl group, a heterocyclic group, an alkenyl group, a cycloalkyl group, a combination thereof, etc. The number of carbon atoms in the substituted amine group is specifically 1 to 30, although not being specially limited thereto. According to an exemplary embodiment, the number of carbon atoms in the amine group is 1 to 20. According to an exemplary embodiment, the number of carbon atoms in the amine group is 1 to 10. Specific examples of the substituted amine groups include a methylamine group, a dimethylamine group, an ethylamine group, a diethylamine group, a phenylamine group, a 9,9-dimethylfluorenylphenylamine group, a pyridylphenylamine group, a diphenylamine group, a phenylpyridylamine group, a naphthylamine group, a biphenylamine group, an anthracenylamine group, a dibenzofuranylphenylamine group, a 9-methylanthracenylamine group, a phenylnaphthylamine group, a ditolylamine group, a phenyltolylamine group, etc., although not being limited thereto.

In the present disclosure, the number of carbon atoms in the aryl group is specifically 6 to 60, although not being specially limited thereto, and it can be a monocyclic aryl group or a polycyclic aryl group. According to an exemplary embodiment, the number of carbon atoms in the aryl group is 6 to 30. According to an exemplary embodiment, the number of carbon atoms in the aryl group is 6 to 20. The aryl group may be a monocyclic aryl group such as a phenyl group, a biphenyl group, a terphenyl group, a quartphenyl group, etc., although not being limited thereto. The polycyclic aryl group may include a naphthyl group, an anthracenyl group, a phenanthrenyl group, a pyrenyl group, a perylenyl group, a triphenyl group, a chrysenyl group, a fluorenyl group, a triphenylenyl group, etc., although not being limited thereto.

In the present disclosure, the fluorenyl group can be substituted, and two substituents can combine to form a spiro structure. The spiro structure can be an aromatic hydrocarbon ring or an aliphatic hydrocarbon ring.

If the fluorene group is substituted, it may have the following structures, although not being limited thereto.

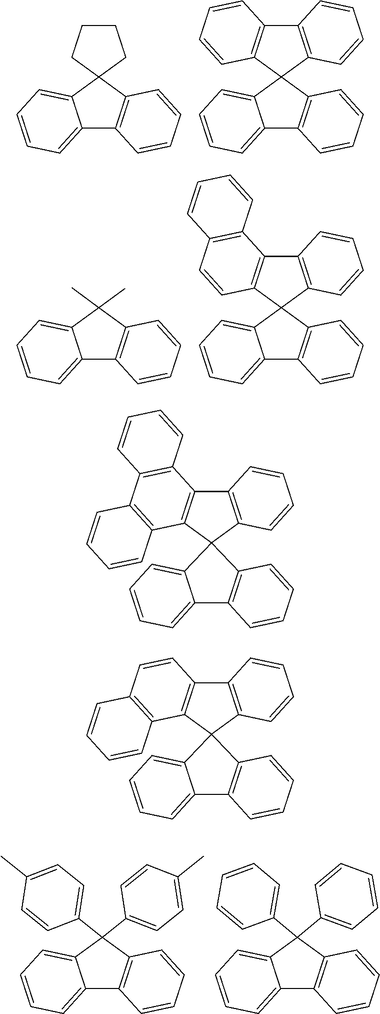

In addition, in the present disclosure, the aryl group may consist only of an aromatic ring, or the aromatic ring may be condensed with an aliphatic ring or it may be substituted with an aliphatic group. The aromatic ring condensed with an aliphatic ring or substituted with an aliphatic group may include a tetrahydronaphthyl group, etc., although not being limited thereto.

In the present disclosure, the foregoing description of the aryl group is applicable to the aryl group in the aryloxy group.

In the present disclosure, the foregoing description of the alkyl group is applicable to the alkyl group in the alkylthioxy group and the alkylsulfoxy group.

In the present disclosure, the foregoing description of the aryl group is applicable to the aryl group of the arylthioxy group and the arylsulfoxy group.

In the present disclosure, the heterocyclic group refers to a cyclic compound that contains one or more heteroatom selected from N, O, P, S, Si and Se. The number of carbon atoms in the heterocyclic group is specifically 2 to 60, although not being specially limited thereto. According to an exemplary embodiment, the number of carbon atoms in the heterocyclic group is 2 to 30. According to an exemplary embodiment, the number of carbon atoms in the heterocyclic group is 2 to 20. Examples of the heterocyclic group include a pyridine group, a pyrrole group, a pyrimidine group, a quinoline group, a pyridazinyl group, a furan group, a thiophene group, an imidazole group, a pyrazole group, a dibenzofuran group, a dibenzothiophene group, a carbazole group, a benzocarbazole group, a naphthobenzofuran group, a benzothiophene group, an indenocarbazole group, a triazinyl group, etc., although not being limited thereto.

In the present disclosure, the foregoing description of the heterocyclic group is applicable to the heteroaryl group, except that it is aromatic.

In the present disclosure, the foregoing description of the aryl group is applicable to the arylene group, except that it is divalent.

In the present disclosure, the foregoing description of the heteroaryl group is applicable to the heteroarylene group, except that it is divalent.

In the present disclosure, in the substituted or unsubstituted ring formed by binding of adjacent groups, the “ring” refers to a hydrocarbon ring; or a heterocycle.

The hydrocarbon ring may be an aromatic ring, an aliphatic ring, or an aromatic or aliphatic condensed ring, and may be selected from the examples of the cycloalkyl group or the aryl group.

In the present disclosure, the formation of a ring by binding of adjacent groups means that a substituted or unsubstituted aliphatic hydrocarbon ring; a substituted or unsubstituted aromatic hydrocarbon ring; a substituted or unsubstituted aliphatic heterocycle; a substituted or unsubstituted aromatic heterocycle; or a condensed ring thereof is formed by binding of adjacent groups. The hydrocarbon ring refers to a ring consisting solely of carbon and hydrogen atoms. The heterocycle refers to a ring that contains one or more element selected from N, O, P, S, Si, Se, etc. In the present disclosure, the aliphatic hydrocarbon ring, the aromatic hydrocarbon ring, the aliphatic heterocycle, and the aromatic heterocycle may be monocyclic or polycyclic.

In the present disclosure, the aliphatic hydrocarbon ring refers to a non-aromatic ring composed solely of carbon and hydrogen atoms, and may include a cycloalkane ring. The foregoing description of the cycloalkyl group is applicable to the cycloalkane ring, except that it is not monovalent. Examples of the aliphatic hydrocarbon ring include cyclopropane, cyclobutane, cyclobutene, cyclopentane, cyclopentene, cyclohexane, cyclohexene, 1,4-cyclohexadiene, cycloheptane, cycloheptene, cyclooctane, cyclooctene, etc., although not being limited thereto.

In the present disclosure, the aromatic hydrocarbon ring refers to an aromatic ring composed solely of carbon and hydrogen atoms. Examples of the aromatic hydrocarbon ring include benzene, naphthalene, anthracene, phenanthrene, perylene, fluoranthene, triphenylene, phenalene, pyrene, tetracene, chrysene, pentacene, fluorene, indene, acenaphthylene, benzofluorene, spirofluorene, etc., although not being limited thereto. In the present disclosure, the aromatic hydrocarbon ring can be interpreted as having the same meaning as the aryl group.

In the present disclosure, the aliphatic heterocycle refers to an aliphatic ring that contains one or more heteroatom. Examples of the aliphatic heterocycle include oxirane, tetrahydrofuran, 1,4-dioxane, pyrrolidine, piperidine, morpholine, oxepane, azocane, thiocane, etc., although not being limited thereto.

In the present disclosure, the aromatic heterocycle refers to an aromatic ring that contains one or more heteroatom. Examples of the aromatic heterocycle include pyridine, pyrrole, pyrimidine, pyridazine, furan, thiophene, imidazole, pyrazole, oxazole, isoxazole, thiazole, isothiazole, triazole, oxadiazole, thiadiazole, dithiazole, tetrazole, pyran, thiopyran, diazine, oxazine, thiazine, dioxin, triazine, tetrazine, isoquinoline, quinoline, quinone, quinazoline, quinoxaline, naphthyridine, acridine, phenanthridine, diazanaphthalene, triazaindene, indole, indolizine, benzothiazole, benzoxazole, benzimidazole, benzothiophene, benzofuran, dibenzothiophene, dibenzofuran, carbazole, benzocarbazole, dibenzocarbazole, phenazine, imidazopyridine, phenoxazine, indolocarbazole, indenocarbazole, etc., although not being limited thereto.

In the present disclosure, the ring formed by bonding of adjacent groups may be one of the following structures.

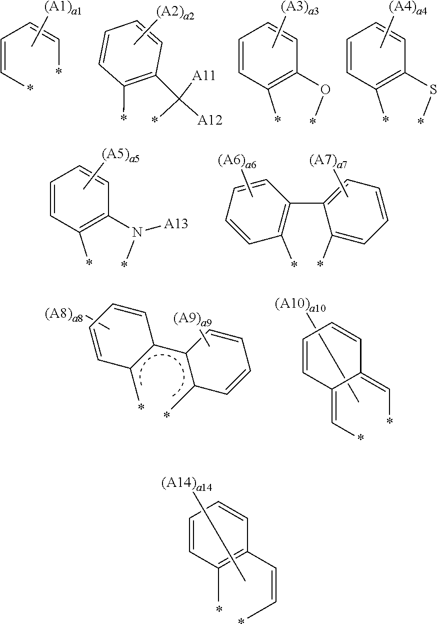

In the above structures,
each of A1 to A14 is independently hydrogen; deuterium; a halogen group; a cyano group; a substituted or unsubstituted alkyl group; a substituted or unsubstituted cycloalkyl group; a substituted or unsubstituted aryl group; or a substituted or unsubstituted heterocyclic group,
each of a1 to a9 is an integer from 1 to 4,
each of a10 and a14 is an integer from 1 to 6,
the two or more substituents within the parentheses are identical or different if each of a1 to a14 is 2 or greater, and
* indicates the position of substitution.

According to an exemplary embodiment of the present disclosure, A1 is hydrogen; or a substituted or unsubstituted aryl group.

According to an exemplary embodiment of the present disclosure, A1 is hydrogen; or a substituted or unsubstituted C₆₋₃₀ aryl group.

According to an exemplary embodiment of the present disclosure, A1 is hydrogen; or a substituted or unsubstituted phenyl group.

According to an exemplary embodiment of the present disclosure, A1 is a hydrogen or a phenyl group.

According to an exemplary embodiment of the present disclosure, A2 to A10 and A14 are hydrogen.

According to an exemplary embodiment of the present disclosure, each of A11 to A13 is independently a substituted or unsubstituted alkyl group; or a substituted or unsubstituted aryl group.

According to an exemplary embodiment of the present disclosure, each of A11 to A13 is independently a substituted or unsubstituted C₁₋₃₀ alkyl group; or a substituted or unsubstituted C₆₋₃₀ aryl group.

According to an exemplary embodiment of the present disclosure, A11 and A12 are methyl groups.

According to an exemplary embodiment of the present disclosure, A13 is a substituted or unsubstituted phenyl group, or a substituted or unsubstituted naphthyl group.

Hereinafter, the specific exemplary embodiments of the present disclosure will be described in detail. However, the exemplary embodiments of the present disclosure can be modified in various forms, and the scope of the present disclosure is not limited to the embodiments described below.

When the compound of Chemical Formula 1 is applied to an organic light-emitting device, an organic light-emitting device with high efficiency, low voltage, and/or long lifespan characteristics can be obtained.

Hereinafter, Chemical Formula 1 will be described in detail.

In the present disclosure, one of R4, R5, R9 and R10 is connected to L of Chemical Formula 2.

In the present disclosure, Chemical Formula 1 is any one of Chemical Formulas 1-1 to 1-4.

In the present disclosure, Chemical Formula 1 is Chemical Formula 1-1.

In the present disclosure, Chemical Formula 1 is Chemical Formula 1-2.

In the present disclosure, Chemical Formula 1 is Chemical Formula 1-3.

In the present disclosure, Chemical Formula 1 is Chemical Formula 1-4.

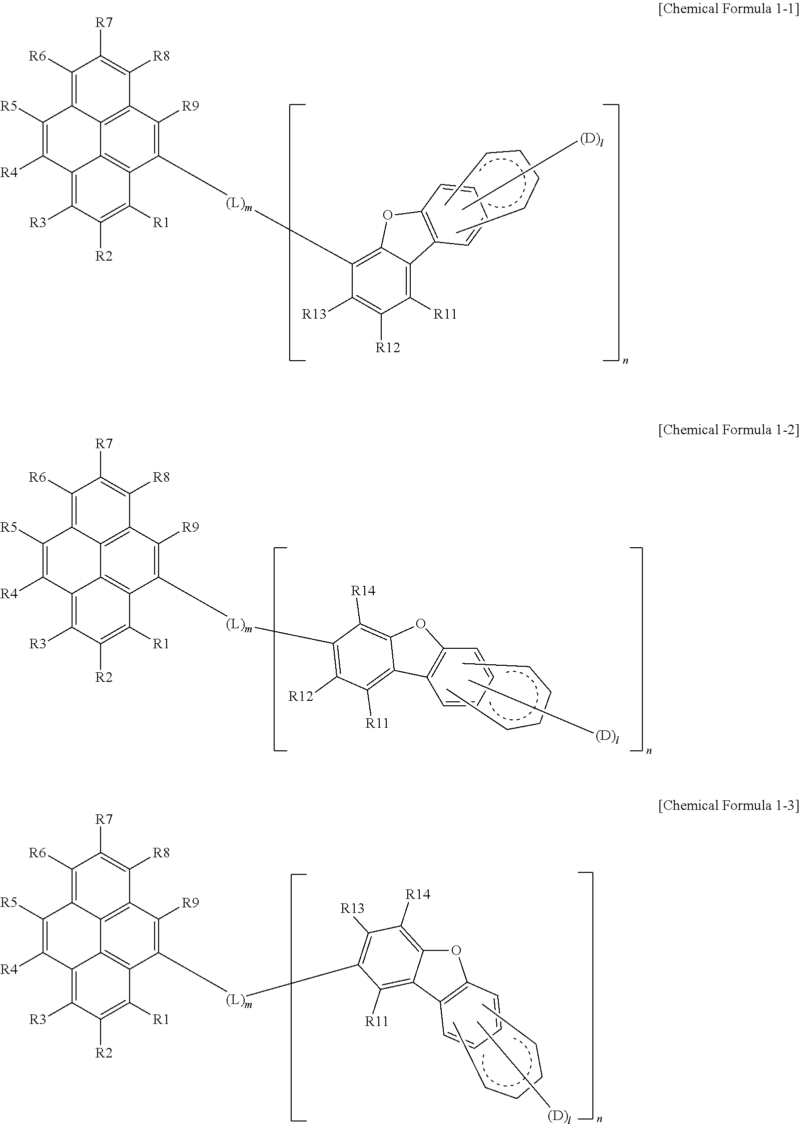

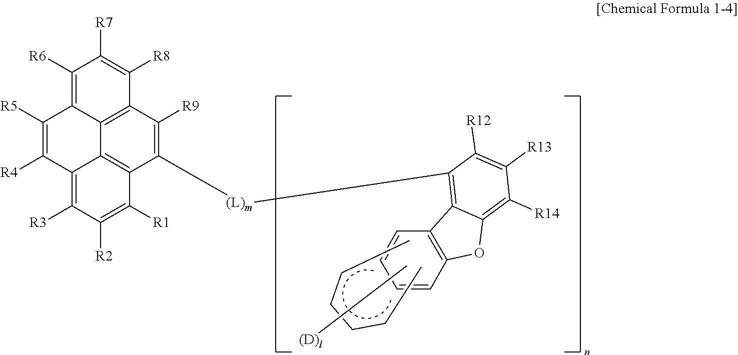

In Chemical Formulas 1-1 to 1-4, L, l, m and n are the same as defined in Chemical Formula 2, and
R1 to R9 and R11 to R14, which are identical or different, are independently hydrogen, deuterium, a substituted or unsubstituted tetrahydronaphthalenyl group, a substituted or unsubstituted silyl group, or a substituted or unsubstituted aryl group.

In the present disclosure, two of R4, R5, R9 and R10 are independently connected to L of Chemical Formula 2.

In the present disclosure, three of R4, R5, R9 and R10 are independently connected to L of Chemical Formula 2.

In the present disclosure, four of R4, R5, R9 and R10 are independently connected to L of Chemical Formula 2.

In the present disclosure, Chemical Formula 1 is any one of Chemical Formulas 1-A to 1-D.

In the present disclosure, Chemical Formula 1 is Chemical Formula 1-A.

In the present disclosure, Chemical Formula 1 is Chemical Formula 1-B.

In the present disclosure, Chemical Formula 1 is Chemical Formula 1-C.

In the present disclosure, Chemical Formula 1 is Chemical Formula 1-D.

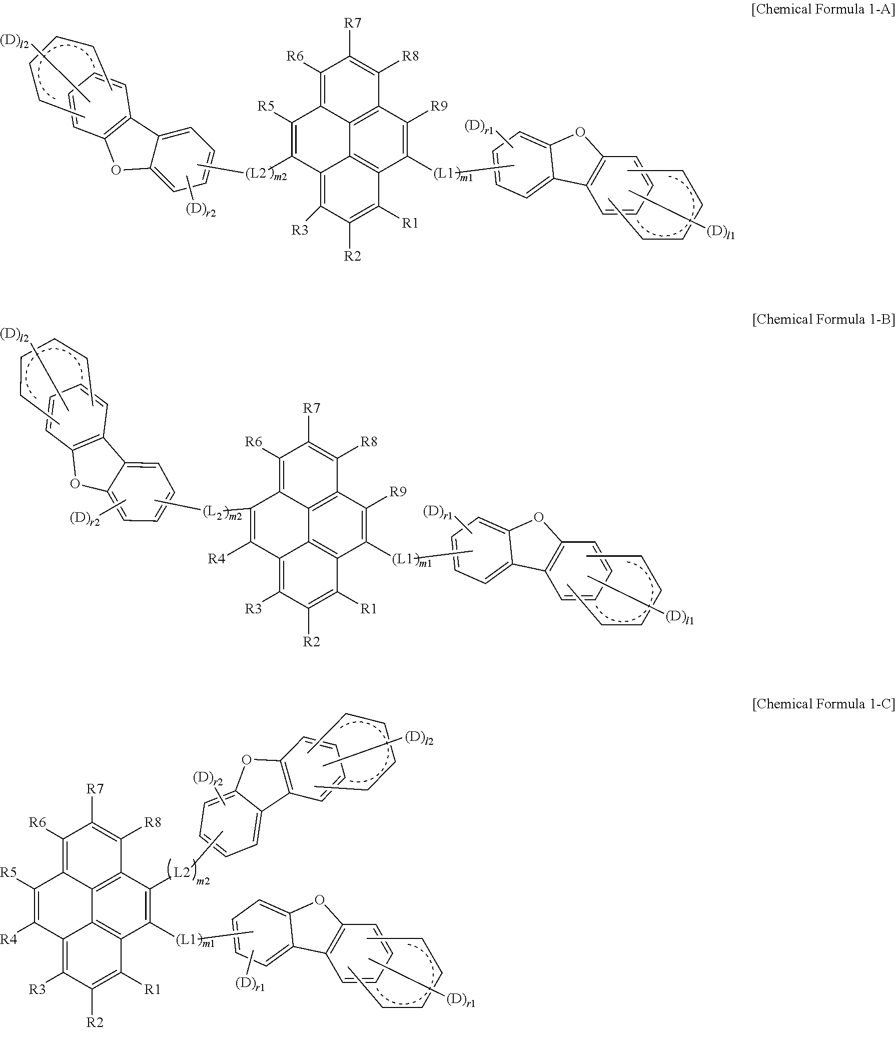

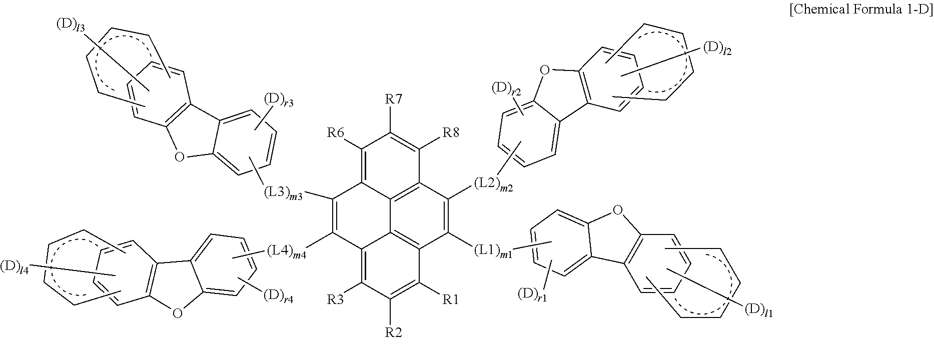

In Chemical Formulas 1-A to 1-D,

R1 to R9, which are identical or different, are independently hydrogen, deuterium, a substituted or unsubstituted tetrahydronaphthalenyl group, a substituted or unsubstituted silyl group, or a substituted or unsubstituted aryl group,

L1 to L4, which are identical or different, are independently a single bond, a substituted or unsubstituted arylene group, or a substituted or unsubstituted divalent heterocyclic group,
each of m1 to m4 is an integer from 1 to 4,
two or more L1s are identical or different if m1 is 2 or greater,
two or more L2s are identical or different if m2 is 2 or greater,
two or more L3s are identical or different if m3 is 2 or greater,
two or more L4s are identical or different if m4 is 2 or greater,
each of l1 to l4 is the number of deuterium, which is an integer from 0 to 6, and
each of r1 to r4 is the number of deuterium, which is an integer from 0 to 3.

In the present disclosure, Chemical Formula 2 is any one of Chemical Formulas 2-1 to 2-3.

In the present disclosure, Chemical Formula 2 is Chemical Formula 2-1.

In the present disclosure, Chemical Formula 2 is Chemical Formula 2-2.

In the present disclosure, Chemical Formula 2 is Chemical Formula 2-3.

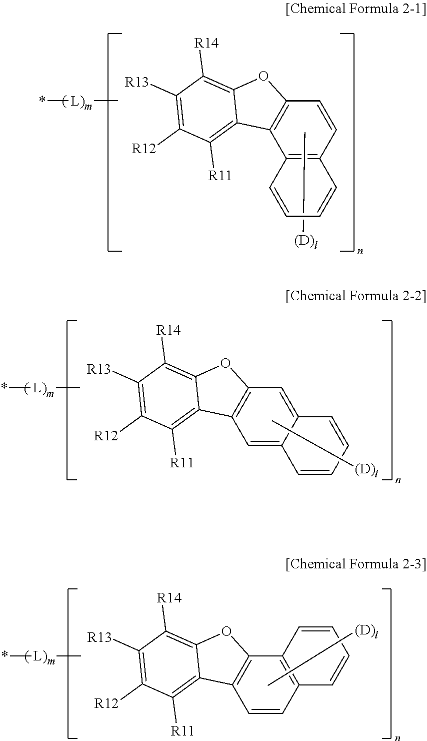

In Chemical Formulas 2-1 to 2-3,1, m, and n are the same as the definition in Chemical Formula 2,
L is a single bond, a substituted or unsubstituted arylene group, or a substituted or unsubstituted divalent heterocyclic group,
L is connected to any one of R11 to R14, and
each of the remainder of R11 to R14 not connected to L is hydrogen, deuterium, or a substituted or unsubstituted aryl group.

In an exemplary embodiment of the present disclosure, in Chemical Formulas 2-1 to 2-3, L is connected to R11 or R14, and each of the remainder of R11 or R14 not connected to L, R12 and R13 is hydrogen, deuterium, or a substituted or unsubstituted aryl group.

In the present disclosure, L is a single bond, an arylene group substituted or unsubstituted with deuterium, or a divalent heterocyclic group substituted or unsubstituted with deuterium.

In the present disclosure, L is a single bond, a phenylene group unsubstituted or substituted with deuterium, a biphenylene group substituted or unsubstituted with deuterium, a terphenylene group substituted or unsubstituted with deuterium, a naphthylenylene group unsubstituted or substituted with deuterium, a dibenzofuranylene group substituted or unsubstituted with deuterium, or a dibenzothiophenylene group substituted or unsubstituted with deuterium.

In the present disclosure, n is 1.

In the present disclosure, the remainder of R4, R5, R9 and R10 not connected to L, R1 to R3, R6 to R8, and the remainder of R11 to R14 not connected to L, which are identical or different, are independently hydrogen, deuterium, a substituted or unsubstituted tetrahydronaphthalenyl group, a substituted or unsubstituted silyl group, or a substituted or unsubstituted aryl group.

In the present disclosure, the remainder of R4, R5, R9 and R10 not connected to L, R1 to R3, R6 to R8, and the remainder of R11 to R14 not connected to L, which are identical or different, are independently hydrogen; deuterium; a tetrahydronaphthalenyl group substituted or unsubstituted with an alkyl group; a silyl group substituted or unsubstituted with a group selected from a group consisting of deuterium, an alkyl group and an aryl group; a phenyl group substituted or unsubstituted with a group selected from a group consisting of deuterium, a trialkylsilyl group, a triarylsilyl group, an alkyl group and an aryl group; a biphenyl group substituted or unsubstituted with a group selected from a group consisting of deuterium, a trialkylsilyl group, a triarylsilyl group, an alkyl group and an aryl group; or a naphthyl group substituted or unsubstituted with a group selected from a group consisting of deuterium, an alkyl group and an aryl group.

In the present disclosure, the remainder of R4, R5, R9 and R10 not connected to L, R1 to R3, R6 to R8, and the remainder of R11 to R14 not connected to L, which are identical or different, are independently hydrogen; deuterium; a tetrahydronaphthalenyl group substituted with a group selected from a group consisting of deuterium and a methyl group; a silyl group substituted or unsubstituted with a group selected from a group consisting of deuterium, a methyl group and a phenyl group; a phenyl group substituted or unsubstituted with a group selected from a group consisting of deuterium, a trimethylsilyl group, a triphenylsilyl group, an alkyl group and an aryl group; a biphenyl group substituted or unsubstituted with a group selected from a group consisting of deuterium, a trimethylsilyl group, a triphenylsilyl group, an alkyl group and an aryl group; or a naphthyl group substituted or unsubstituted with a group selected from a group consisting of deuterium, an alkyl group and an aryl group.

In the present disclosure, the compound of Chemical Formula 1 is any one of the following structures.

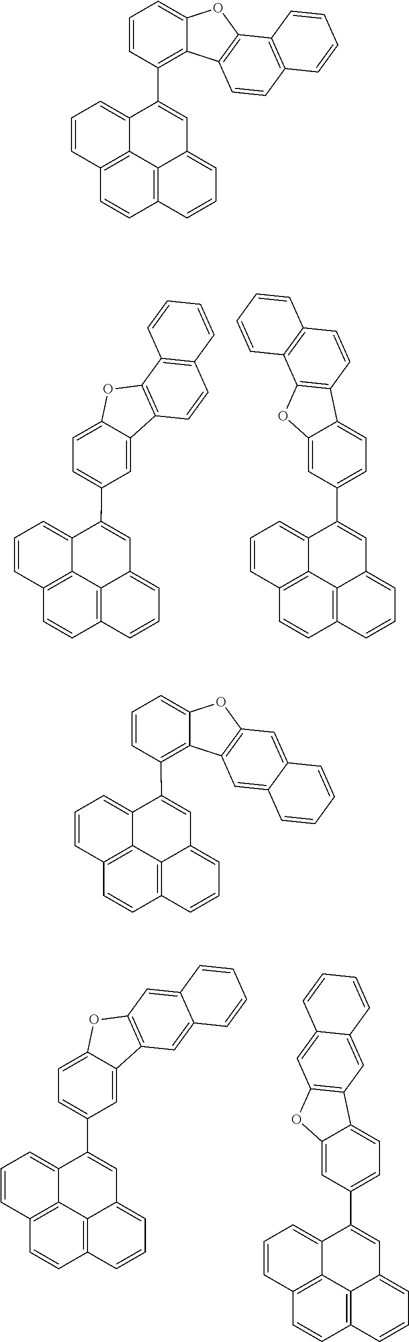

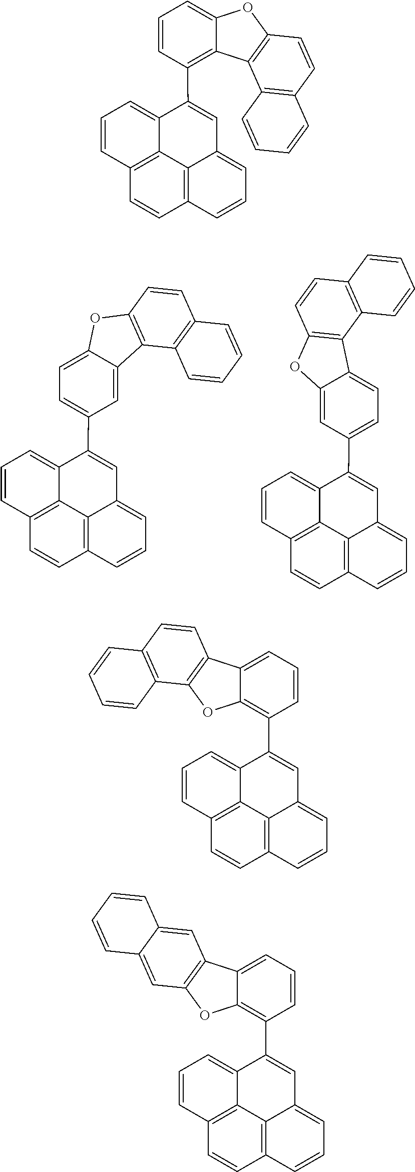 [SMILES: c1ccc2c(c1)ccc1oc3cccc(-c4cc5cccc6ccc7cccc4c7c65)c3c12 ; c1ccc2c(c1)ccc1oc3ccc(-c4cc5cccc6ccc7cccc4c7c65)cc3c12 ; c1ccc2c(c1)ccc1oc3cc(-c4cc5cccc6ccc7cccc4c7c65)ccc3c12 ; c1ccc2c(c1)ccc1c3cccc(-c4cc5cccc6ccc7cccc4c7c65)c3oc21 ; c1ccc2cc3c(cc2c1)oc1c(-c2cc4cccc5ccc6cccc2c6c54)cccc13]

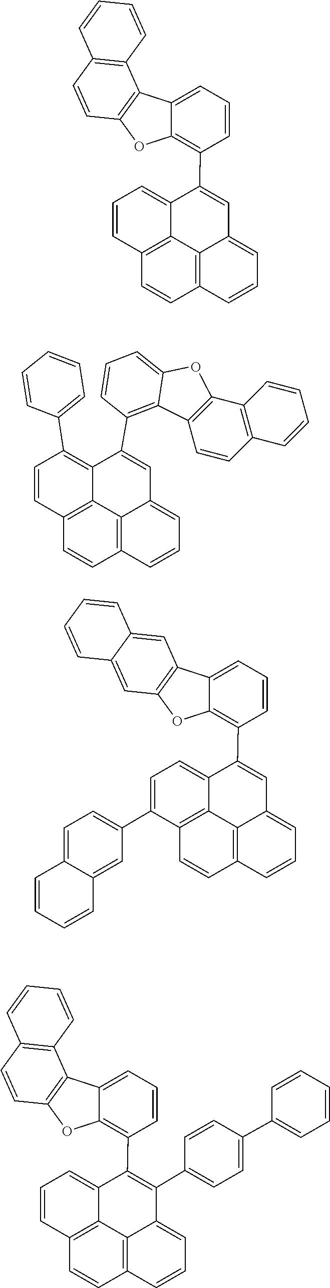

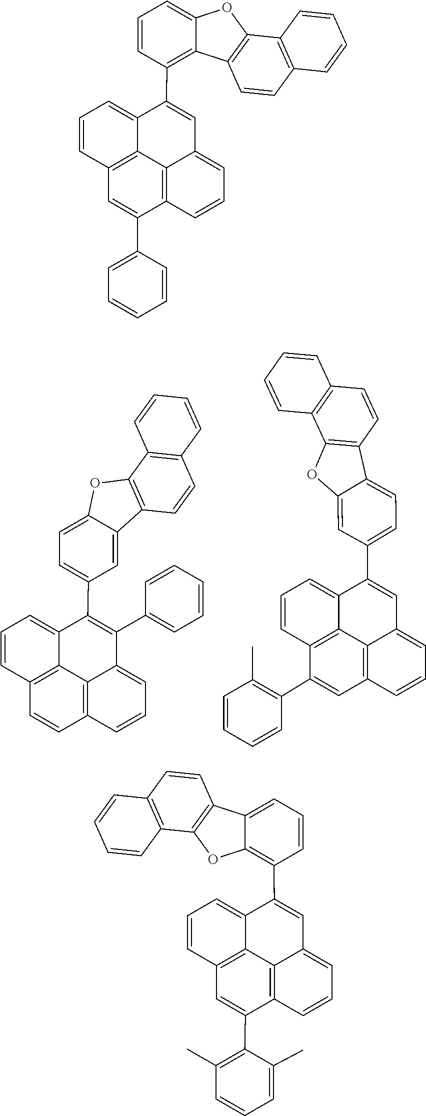 [SMILES: c1ccc(-c2cc3cccc4c(-c5cccc6oc7c8ccccc8ccc7c56)cc5cccc2c5c34)cc1 ; c1ccc(-c2c(-c3ccc4oc5c6ccccc6ccc5c4c3)c3cccc4ccc5cccc2c5c43)cc1 ; Cc1ccccc1-c1cc2cccc3cc(-c4ccc5c(c4)oc4c6ccccc6ccc54)c4cccc1c4c32 ; Cc1cccc(C)c1-c1cc2cccc3c(-c4cccc5c4oc4c6ccccc6ccc54)cc4cccc1c4c23]

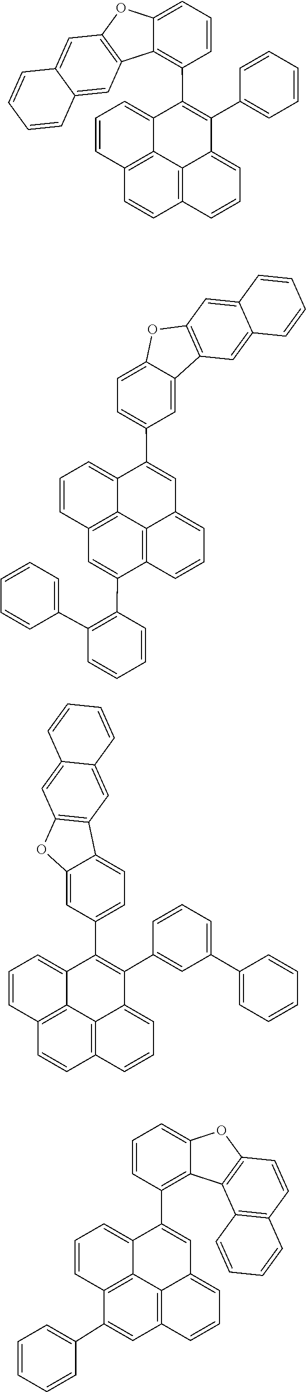

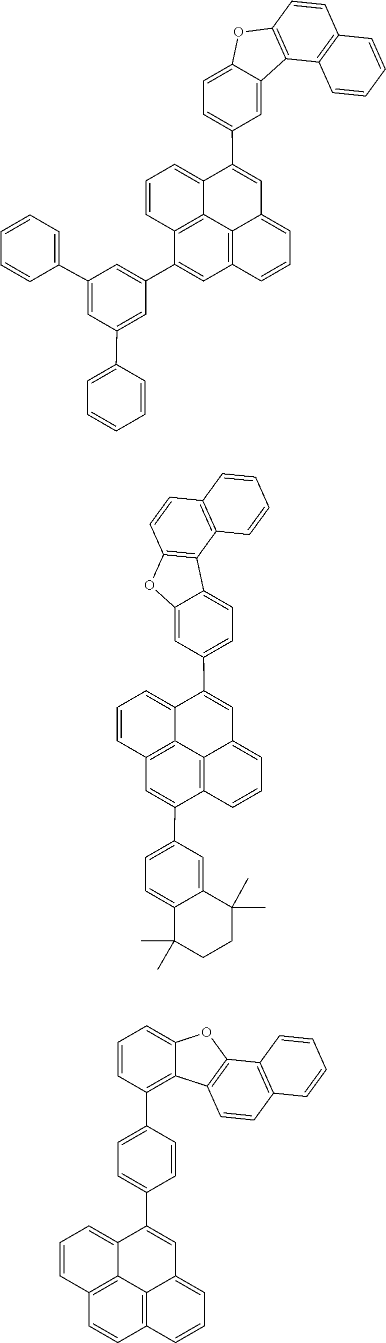 [SMILES: c1ccc(-c2cc(-c3ccccc3)cc(-c3cc4cccc5cc(-c6ccc7oc8ccc9ccccc9c8c7c6)c6cccc3c6c45)c2)cc1 ; CC1(C)CCC(C)(C)c2cc(-c3cc4cccc5c(-c6ccc7c(c6)oc6ccc8ccccc8c67)cc6cccc3c6c45)ccc21 ; c1ccc2c(c1)ccc1c2oc2cccc(-c3ccc(-c4cc5cccc6ccc7cccc4c7c65)cc3)c21]

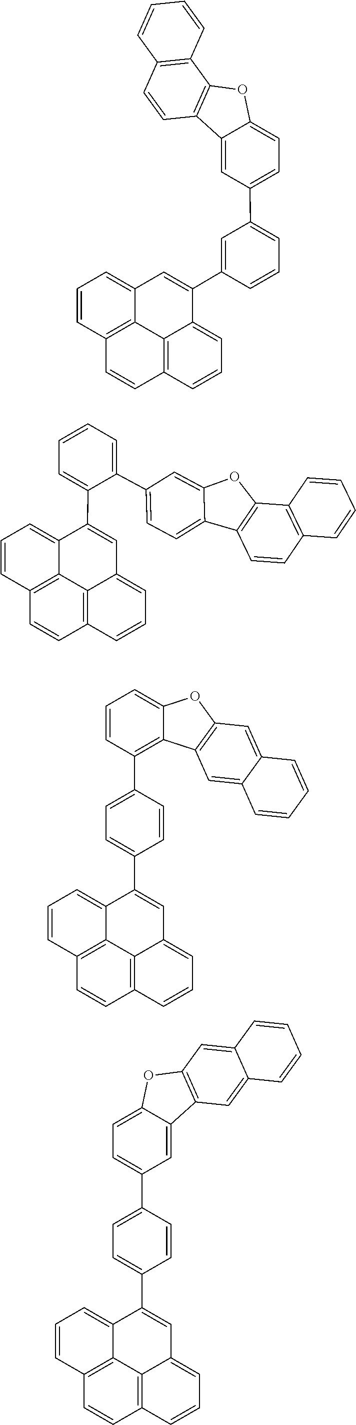 [SMILES: c1cc(-c2ccc3oc4c5ccccc5ccc4c3c2)cc(-c2cc3cccc4ccc5cccc2c5c43)c1 ; c1ccc(-c2cc3cccc4ccc5cccc2c5c43)c(-c2ccc3c(c2)oc2c4ccccc4ccc32)c1 ; c1ccc2cc3c(cc2c1)oc1cccc(-c2ccc(-c4cc5cccc6ccc7cccc4c7c65)cc2)c13 ; c1ccc2cc3c(cc2c1)oc1ccc(-c2ccc(-c4cc5cccc6ccc7cccc4c7c65)cc2)cc13]

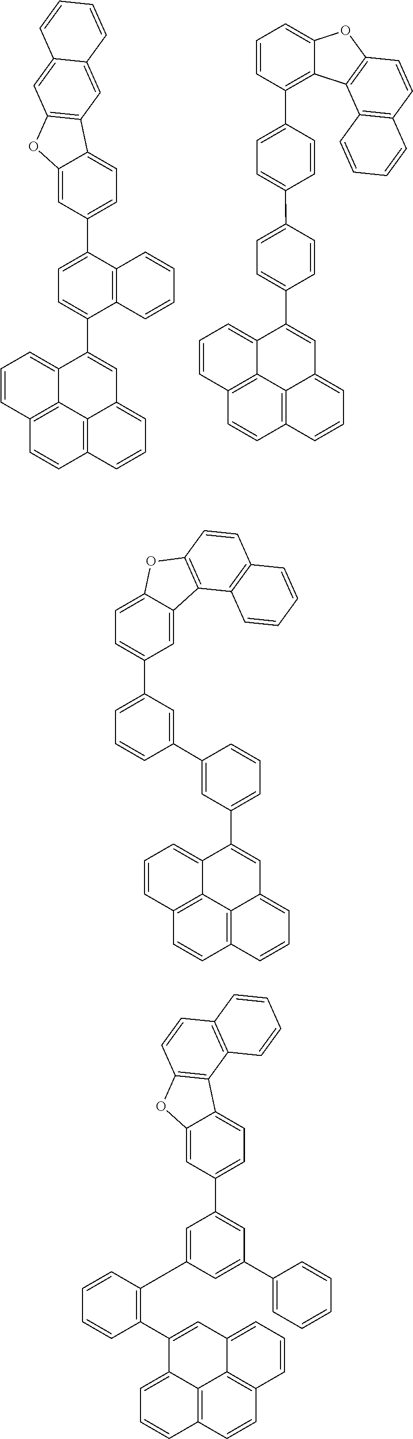

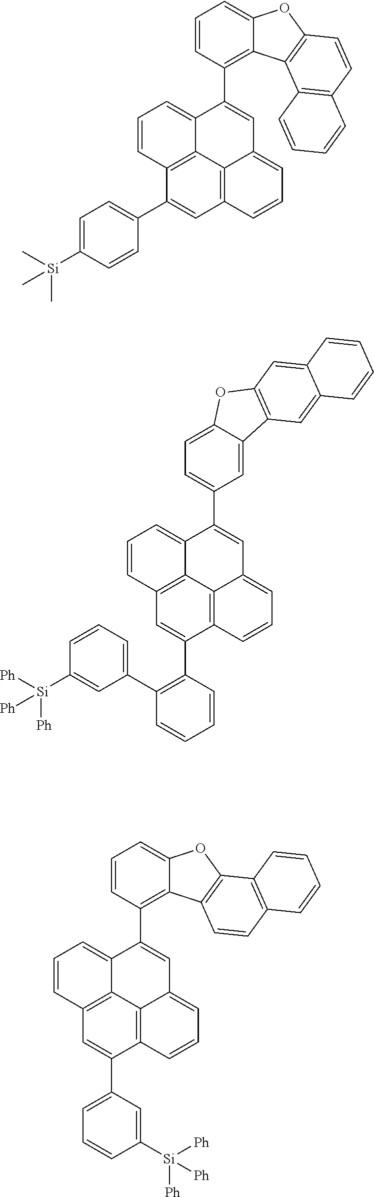 [SMILES: C[Si](C)(C)c1ccc(-c2cc3cccc4cc(-c5cccc6oc7ccc8ccccc8c7c56)c5cccc2c5c34)cc1 ; c1ccc([Si](c2ccccc2)(c2ccccc2)c2cccc(-c3ccccc3-c3cc4cccc5c(-c6ccc7oc8cc9ccccc9cc8c7c6)cc6cccc3c6c45)c2)cc1 ; c1ccc([Si](c2ccccc2)(c2ccccc2)c2cccc(-c3cc4cccc5c(-c6cccc7oc8c9ccccc9ccc8c67)cc6cccc3c6c45)c2)cc1]

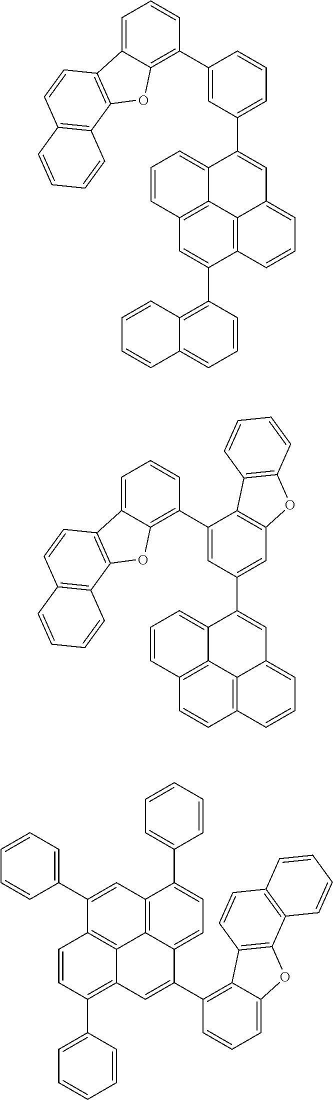 [SMILES: c1cc(-c2cc3cccc4c(-c5cccc6ccccc56)cc5cccc2c5c34)cc(-c2cccc3c2oc2c4ccccc4ccc32)c1 ; c1ccc2c(c1)ccc1c3cccc(-c4cc(-c5cc6cccc7ccc8cccc5c8c76)cc5oc6ccccc6c45)c3oc21 ; c1ccc(-c2cc3c(-c4ccccc4)ccc4c(-c5cccc6oc7c8ccccc8ccc7c56)cc5c(-c6ccccc6)ccc2c5c34)cc1]

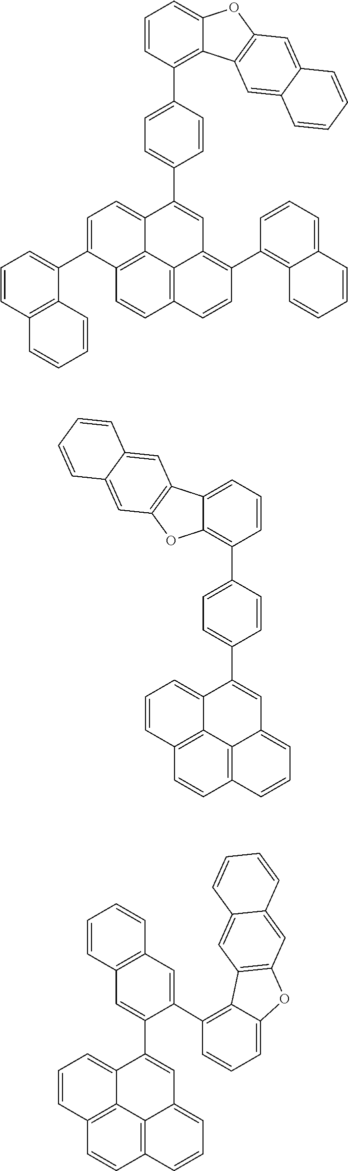 [SMILES: c1ccc2cc3c(cc2c1)oc1cccc(-c2ccc(-c4cc5c(-c6cccc7ccccc67)ccc6ccc7c(-c8cccc9ccccc89)ccc4c7c65)cc2)c13 ; c1ccc2cc3c(cc2c1)oc1c(-c2ccc(-c4cc5cccc6ccc7cccc4c7c65)cc2)cccc13 ; c1ccc2cc(-c3cccc4oc5cc6ccccc6cc5c34)c(-c3cc4cccc5ccc6cccc3c6c54)cc2c1]

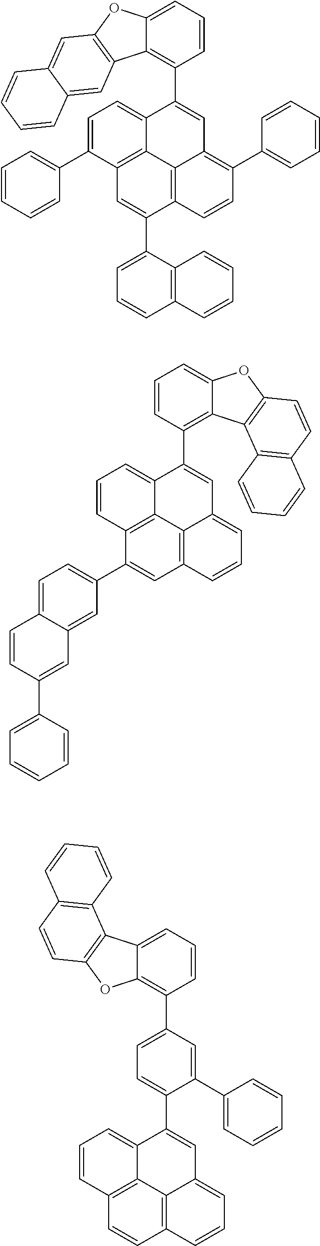 [SMILES: c1ccc(-c2ccc3c(-c4cccc5ccccc45)cc4c(-c5ccccc5)ccc5c(-c6cccc7oc8cc9ccccc9cc8c67)cc2c3c45)cc1 ; c1ccc(-c2ccc3ccc(-c4cc5cccc6cc(-c7cccc8oc9ccc%10ccccc%10c9c78)c7cccc4c7c56)cc3c2)cc1 ; c1ccc(-c2cc(-c3cccc4c3oc3ccc5ccccc5c34)ccc2-c2cc3cccc4ccc5cccc2c5c43)cc1]

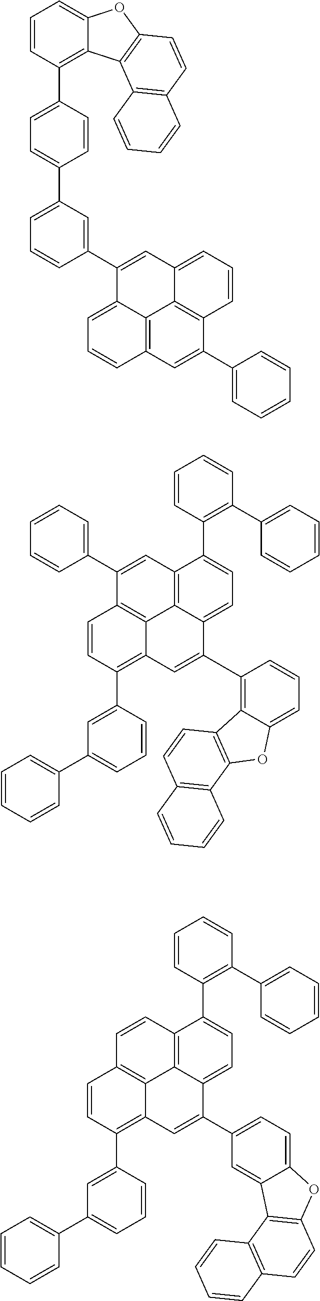 [SMILES: c1ccc(-c2cc3cccc4c(-c5cccc(-c6ccc(-c7cccc8oc9ccc%10ccccc%10c9c78)cc6)c5)cc5cccc2c5c34)cc1 ; c1ccc(-c2cccc(-c3ccc4c(-c5ccccc5)cc5c(-c6ccccc6-c6ccccc6)ccc6c(-c7cccc8oc9c%10ccccc%10ccc9c78)cc3c4c56)c2)cc1 ; c1ccc(-c2cccc(-c3ccc4ccc5c(-c6ccccc6-c6ccccc6)ccc6c(-c7ccc8oc9ccc%10ccccc%10c9c8c7)cc3c4c65)c2)cc1]

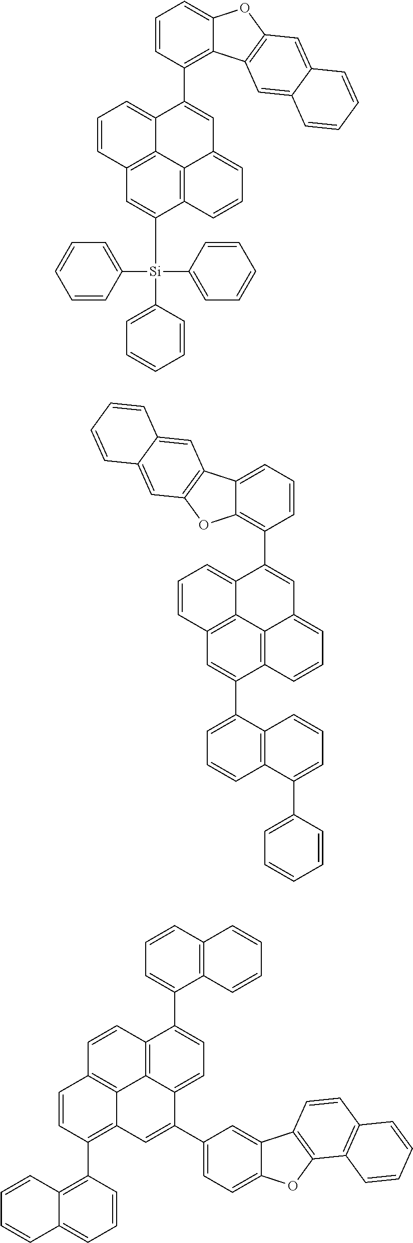

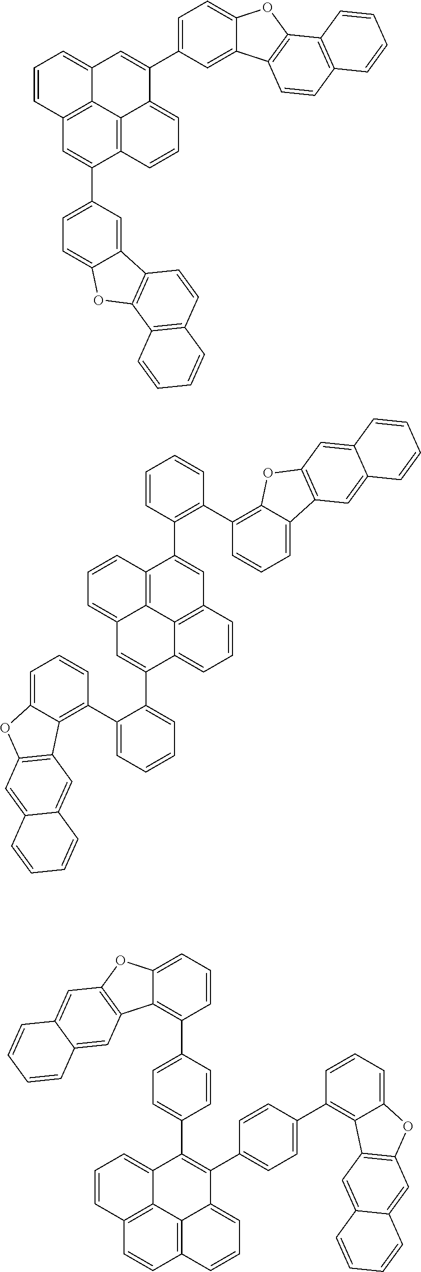 [SMILES: c1ccc2c(c1)ccc1c3cc(-c4cc5cccc6cc(-c7ccc8oc9c%10ccccc%10ccc9c8c7)c7cccc4c7c56)ccc3oc21 ; c1ccc(-c2cccc3c2oc2cc4ccccc4cc23)c(-c2cc3cccc4c(-c5ccccc5-c5cccc6oc7cc8ccccc8cc7c56)cc5cccc2c5c34)c1 ; c1ccc2cc3c(cc2c1)oc1cccc(-c2ccc(-c4c(-c5ccc(-c6cccc7oc8cc9ccccc9cc8c67)cc5)c5cccc6ccc7cccc4c7c65)cc2)c13]

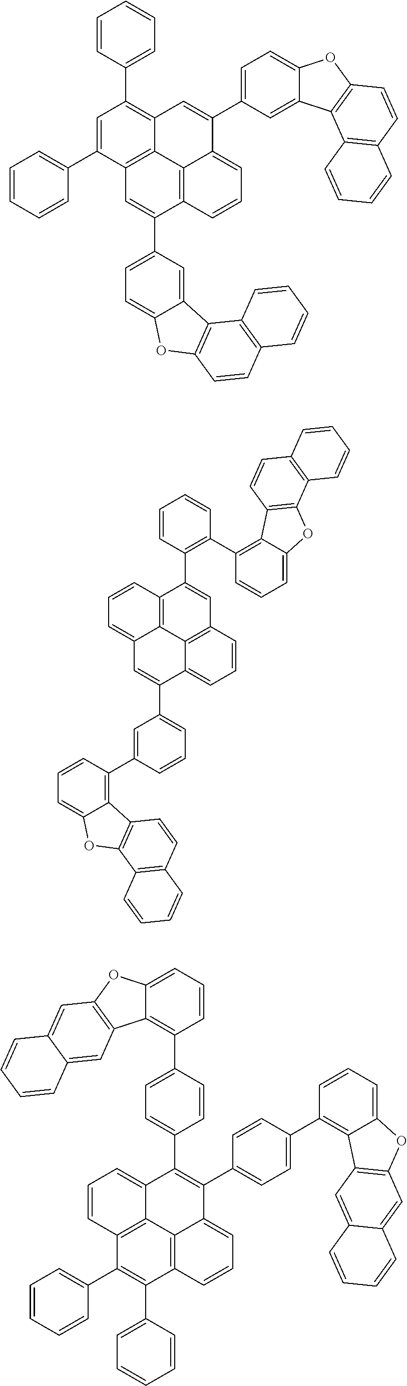 [SMILES: c1ccc(-c2cc(-c3ccccc3)c3cc(-c4ccc5oc6ccc7ccccc7c6c5c4)c4cccc5c(-c6ccc7oc8ccc9ccccc9c8c7c6)cc2c3c54)cc1 ; c1cc(-c2cc3cccc4c(-c5ccccc5-c5cccc6oc7c8ccccc8ccc7c56)cc5cccc2c5c34)cc(-c2cccc3oc4c5ccccc5ccc4c23)c1 ; c1ccc(-c2c(-c3ccccc3)c3cccc4c(-c5ccc(-c6cccc7oc8cc9ccccc9cc8c67)cc5)c(-c5ccc(-c6cccc7oc8cc9ccccc9cc8c67)cc5)c5cccc2c5c34)cc1]

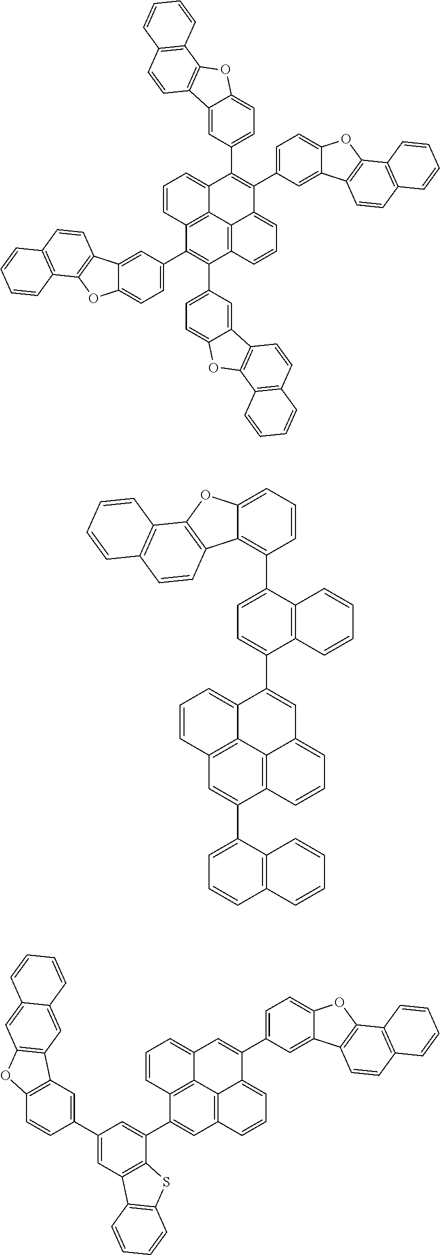 [SMILES: c1ccc2c(c1)ccc1c3cc(-c4c(-c5ccc6oc7c8ccccc8ccc7c6c5)c5cccc6c(-c7ccc8oc9c%10ccccc%10ccc9c8c7)c(-c7ccc8oc9c%10ccccc%10ccc9c8c7)c7cccc4c7c56)ccc3oc21 ; c1ccc2c(-c3cc4cccc5c(-c6ccc(-c7cccc8oc9c%10ccccc%10ccc9c78)c7ccccc67)cc6cccc3c6c45)cccc2c1 ; c1ccc2cc3c(cc2c1)oc1ccc(-c2cc(-c4cc5cccc6c(-c7ccc8oc9c%10ccccc%10ccc9c8c7)cc7cccc4c7c56)c4sc5ccccc5c4c2)cc13]

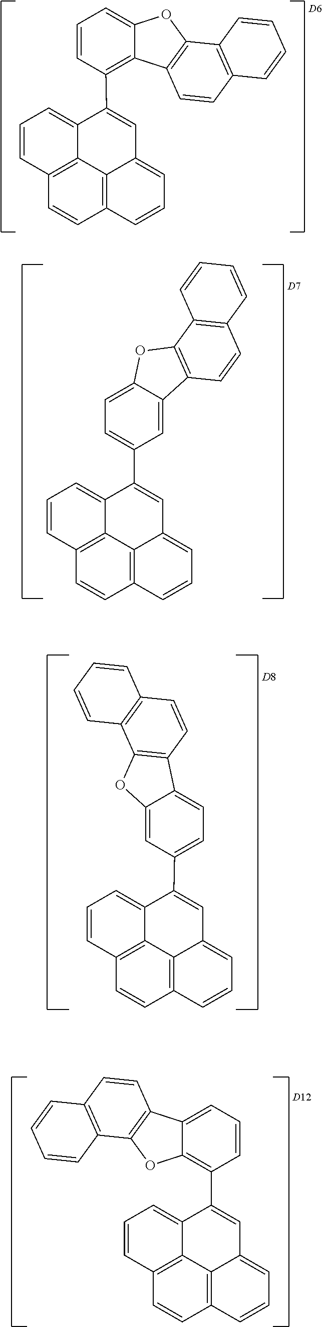

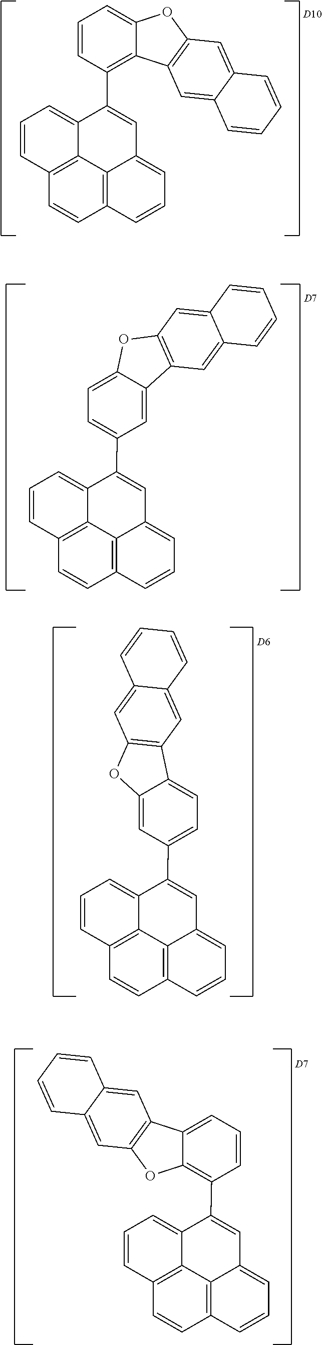

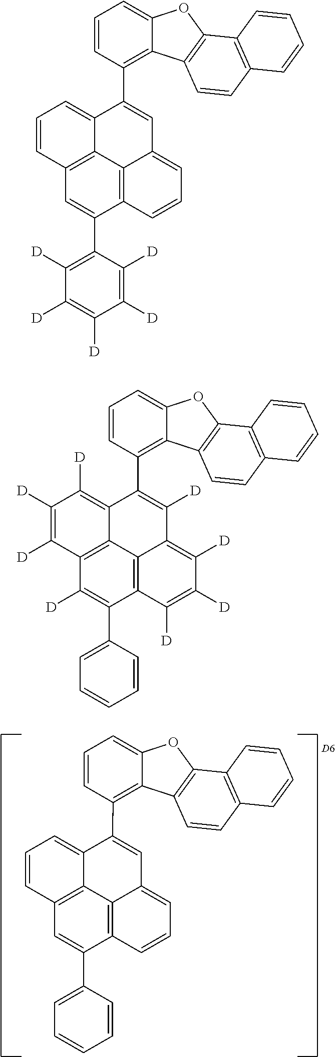

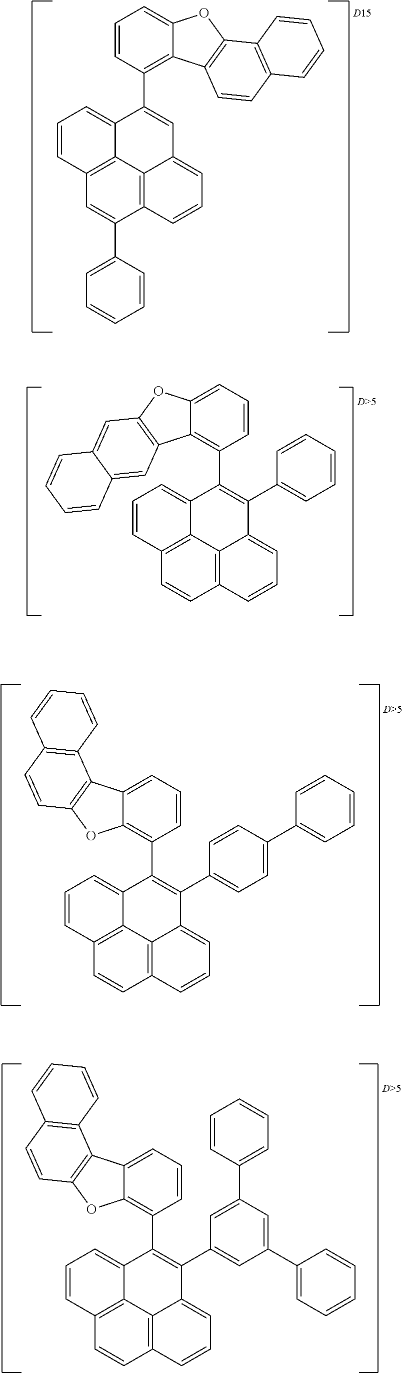

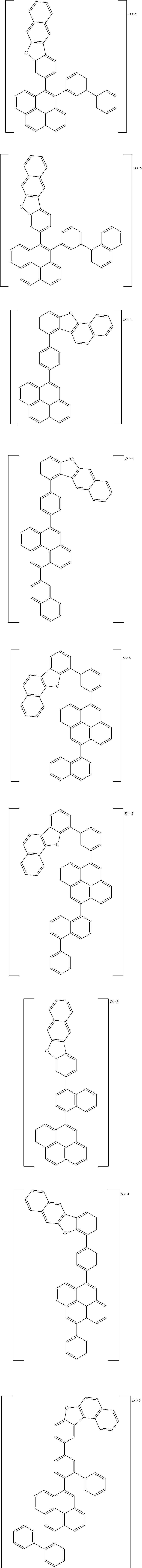

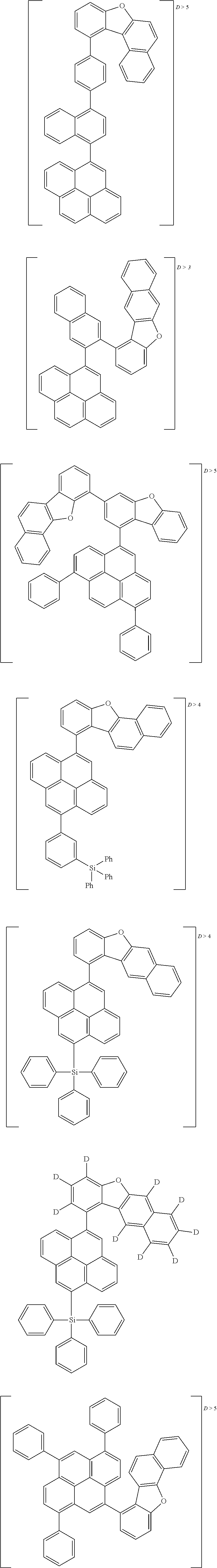

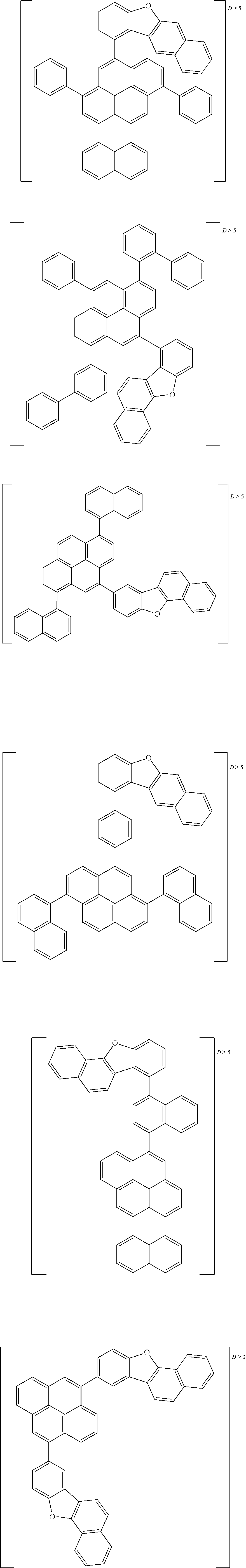

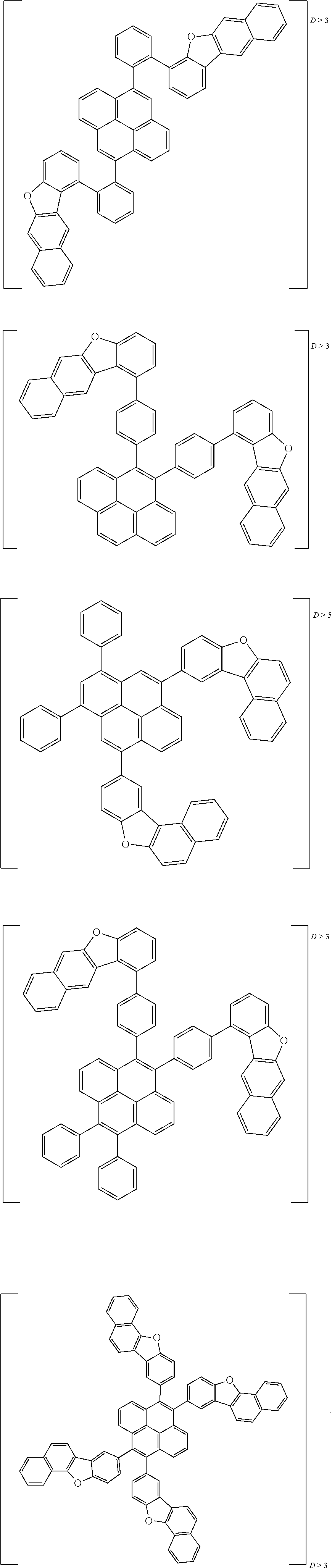

DN refers to a compound that has N deuterium atoms. That is to say, N is an integer representing the number of deuterium atoms. D>N means that the compound is substituted with deuterium atoms more than any arbitrary number N, which indicates the maximum number of deuterium that can be substituted within the structures within the parentheses.

The compound of Chemical Formula 1 according to an exemplary embodiment of the present disclosure can be prepared into a core structure in a manner as described in the preparation examples below. Other substitutes can be combined by methods known in the technical field, and the type, position or number of the substitutes can be changed according to the technology known in the technical field.

In the present disclosure, compounds with various energy band gaps can be synthesized by introducing various substituents into the core structure of the compound of Chemical Formula 1. In addition, in the present disclosure, the HOMO and LUMO energy levels of the compounds can be adjusted by introducing various substituents into the core structure.

In addition, the present disclosure provides an organic light-emitting device that contains the compound described above.

The organic light-emitting device according to the present disclosure includes an anode; a cathode; and one or more organic layer provided between the anode and the cathode, wherein the one or more of organic layer contains the compound of Chemical Formula 1.

The organic light-emitting device of the present disclosure can be prepared using materials and methods known in the art, except that at least one layer of the organic material layer contains the compound of Chemical Formula 1.

When the organic light-emitting includes a plurality of organic layers, the organic layers can be formed of identical or different materials.

For example, the organic light-emitting device according to the present disclosure may be prepared by using the PVD (physical vapor deposition) method such as sputtering or e-beam evaporation to deposit a metal, a conductive metal oxide or an alloy thereof on a substrate to form an anode, and then forming an organic layer that includes a hole injection layer, a hole transport layer, a light-emitting layer, an electron blocking (hole controlling) layer, an electron transport layer, and an electron injection layer, and then depositing a material that can be used as a cathode on top of that. In addition to this method, an organic light-emitting device can also be prepared by sequentially depositing a cathode material, an organic layer, and an anode material on a substrate.

In addition, when the organic layer of the organic light-emitting device contains two or more compounds, it can be deposited using a co-deposition or pre-mix method.

The organic layer of the organic light-emitting device in the present disclosure may consist of a single-layer structure, but it can also be composed of a multilayer structure wherein two or more organic layers are stacked. For example, the organic light-emitting device of the present disclosure may have a structure that includes one or more layer selected from a hole transport layer, a hole injection layer, an electron blocking (hole control) layer, a hole transport and injection layer, a light-emitting layer, an electron transport layer, an electron injection layer, a hole blocking (electron control) layer, and an electron transport and injection layer, as an organic layer. However, the structure of the organic light-emitting device in the present disclosure is not limited to thereto and may include fewer or more organic layers.

In addition, the organic layer can be prepared with fewer layers using various polymer materials through a solvent process such as spin coating, dip coating, doctor blading, screen printing, inkjet printing, thermal-transfer printing, etc., rather than the deposition method.

In the organic light-emitting device of the present disclosure, the organic layer includes a light-emitting layer, and the light-emitting layer contains the compound of Chemical Formula 1.

The light-emitting layer contains the compound of Chemical Formula 1 as a host of the light-emitting layer, and may contain an additional host and a dopant. The additional host may be contained in the organic layer containing the compound of Chemical Formula 1 mentioned above, and may also be contained in another adjacent organic layer.

In the organic light-emitting device of the present disclosure, the light-emitting layer may additionally contain an additional host and a dopant, and the total hosts and dopants may be contained at a weight ratio of 99:1 to 1:99, specifically 99:1 to 70:30, more specifically 99:1 to 85:15.

The organic light-emitting device of the present disclosure may further include one or more organic layer selected from a hole transport layer, a hole injection layer, an electron blocking (hole control) layer, an electron injection and transport layer, an electron transport layer, an electron injection layer, a hole blocking (electron control) layer, and a hole injection and transport layer.

The organic layer may further include one or more layer selected from a hole transport layer, a hole injection layer, an electron blocking (hole control) layer, an electron transport and injection layer, an electron transport layer, an electron injection layer, a hole blocking (electron control) layer, and a hole injection and transport layer.

In an exemplary embodiment of the present disclosure, the organic light-emitting device includes an anode; a cathode; and an organic layer of two or more layers provided between the anode and the cathode, wherein at least one of the two or more organic layers contains the compound of Chemical Formula 1.

In an exemplary embodiment of the present disclosure, the organic light-emitting device may be an organic light-emitting device with a normal type structure in which an anode, one or more organic layer, and a cathode are stacked sequentially on a substrate.

In an exemplary embodiment of the present disclosure, the organic light-emitting device may be an organic light-emitting device with an inverted type structure in which a cathode, one or more organic layer, and an anode are stacked sequentially on a substrate.

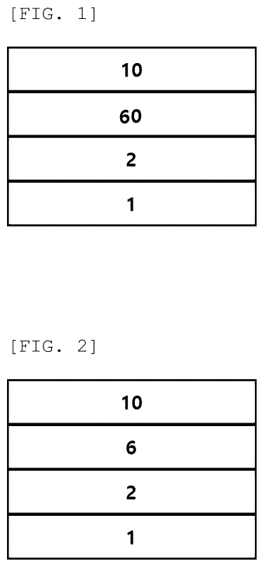

The organic light-emitting device in the present disclosure may have the structures shown in FIGS. 1 to 7, although not being limited thereto.

FIG. 1 shows the structure of an exemplary organic light-emitting device in which a substrate 1, an anode 2, an organic layer 60, and a cathode 10 are stacked sequentially. In such a structure, the compound of Chemical Formula 1 can be contained in the organic layer 60 of one or more layers.

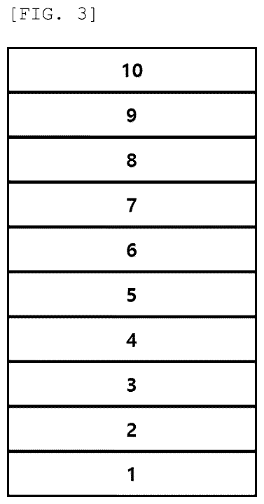

FIG. 2 shows the structure of an exemplary organic light-emitting device in which a substrate 1, an anode 2, a light-emitting layer 6, and a cathode 10 are stacked sequentially. In such a structure, the compound of Chemical Formula 1 can be contained in the light-emitting layer 6.

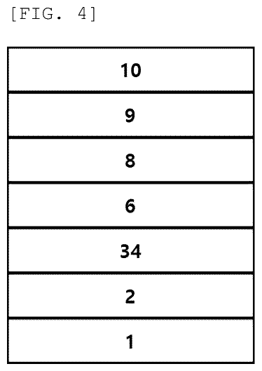

FIG. 3 shows the structure of an exemplary organic light-emitting device in which a substrate 1, an anode 2, a hole injection layer 3, a hole transport layer 4, an electron blocking (hole control) layer 5, a light-emitting layer 6, a hole blocking (electron control) layer 7, an electron transport layer 8, an electron injection layer 9, and a cathode 10 are stacked sequentially. In such a structure, the compound of Chemical Formula 1 can be contained in the light-emitting layer 6.

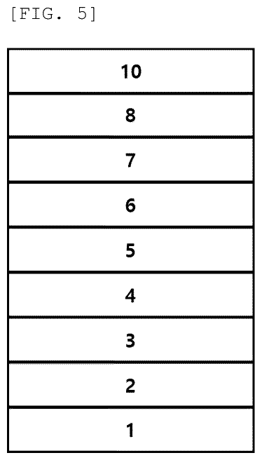

FIG. 4 shows the structure of an exemplary organic light-emitting device in which a substrate 1, an anode 2, a hole injection and transport layer 34, a light-emitting layer 6, an electron transport layer 8, an electron injection layer 9, and a cathode 10 are stacked sequentially. In such a structure, the compound of Chemical Formula 1 can be contained in the light-emitting layer 6.

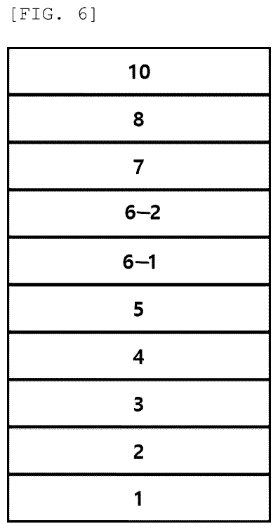

FIG. 5 shows the structure of an exemplary organic light-emitting device in which a substrate 1, an anode 2, a hole injection layer 3, a hole transport layer 4, an electron blocking (hole control) layer 5, a light-emitting layer 6, a hole blocking (electron control) layer 7, an electron transport layer 8, and a cathode 10 are stacked sequentially. In such a structure, the compound of Chemical Formula 1 can be contained in the light-emitting layer 6.

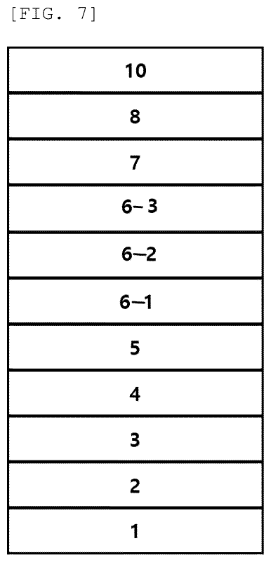

FIG. 6 shows the structure of an exemplary organic light-emitting device in which a substrate 1, an anode 2, a hole injection layer 3, a hole transport layer 4, an electron blocking (hole control) layer 5, a first light-emitting layer 6-1, a second light-emitting layer 6-2, a hole blocking (electron control) layer 7, an electron transport layer 8, and a cathode 10 are stacked sequentially. In such a structure, the compound of Chemical Formula 1 can be contained in the first light-emitting layer 6-1.

FIG. 7 shows the structure of an exemplary organic light-emitting device in which a substrate 1, an anode 2, a hole injection layer 3, a hole transport layer 4, an electron blocking (hole control) layer 5, a first light-emitting layer 6-1, a second light-emitting layer 6-2, a third light-emitting layer 6-3, a hole blocking (electron control) layer 7, an electron transport layer 8, and a cathode 10 are stacked sequentially. In such a structure, the compound of Chemical Formula 1 can be contained in the first light-emitting layer 6-1 and can also be further contained in the third light-emitting layer 6-3.

In an exemplary embodiment of the present disclosure, the electronic blocking (hole control) layer and the light-emitting layer may be provided to be adjacent to each other. For example, the electronic blocking (hole control) layer and the light-emitting layer may be provided to be in physical contact with each other.

In an exemplary embodiment of the present disclosure, the hole transport layer and the electron blocking (hole control) layer may be provided to be adjacent to each other. For example, the above-mentioned hole transport layer and electron blocking (hole control) layer may be provided to be in physical contact with each other.

The anode is an electrode which injects holes, and a material with a large work function is typically preferred as an anode material, so that hole injection into the organic layer can occur smoothly. Specific examples of the anode material that can be used in the present disclosure include metals such as vanadium, chromium, copper, zinc and gold, or their alloys; metal oxides such as zinc oxide, indium oxide, indium tin oxide (ITO) and indium zinc oxide (IZO); combinations of metals and oxides such as ZnO:Al or Snl₂:Sb; conductive polymers such as poly(3-methylthiophene), poly[3,4-(ethylene-1,2-dioxy)thiophene] (PEDOT), polypyrrole and polyaniline, etc., although not being limited thereto.

The cathode is an electrode which injects electrons, and a material with a small work function is typically preferred as a cathode material, so that electron injection into the organic layer can occur smoothly. Specific examples of the cathode material include metals such as magnesium, calcium, sodium, potassium, titanium, indium, yttrium, lithium, gadolinium, aluminum, silver, tin and lead or their alloys; multilayer-structured materials such as LiF/Al or LiO₂/Al, etc., although not being limited thereto.

The hole injection layer can facilitate the injection of holes from the anode to the light-emitting layer. As a hole injection material, a material that can receive holes well from the anode at low voltage, with the HOMO (highest occupied molecular orbital) of the hole injection material between the work function of the anode and the HOMO of the surrounding organic layer, is preferred. Specific examples of the hole injection material include metal porphyrins, oligothiophenes, arylamine-based compounds, hexanitrilehexaazatriphenylene-based compounds, quinacridone-based compounds, perylene-based compounds, benzonitrile-based compounds, anthraquinone, polyaniline- and polythiophene-based conductive polymers, etc., although not being limited thereto. Specifically, arylamine-based compounds and benzonitrile-based compounds may be used as the hole injection layer.

In the organic light-emitting device of the present disclosure, a carbazole-substituted arylamine-based compound and a halogen-substituted benzonitrile-based compound may be used in the hole injection layer, although not being limited thereto.

In an exemplary embodiment of the present disclosure, the hole injection layer contains a compound of Chemical Formula HI-1.

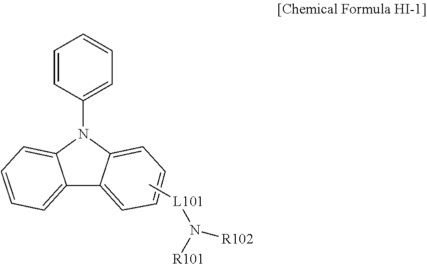

In Chemical Formula HI-1,
L101 is a direct bond; or a substituted or unsubstituted arylene group, and
R101 and R102, which are identical or different, are independently hydrogen; deuterium; a substituted or unsubstituted alkyl group; a substituted or unsubstituted amine group; a substituted or unsubstituted aryl group; or a substituted or unsubstituted heterocyclic group.

In an exemplary embodiment of the present disclosure, L101 is a substituted or unsubstituted phenylene group; or a substituted or unsubstituted naphthylene group.

In an exemplary embodiment of the present disclosure, L101 is a substituted or unsubstituted phenylene group.

In an exemplary embodiment of the present disclosure, L101 is a phenylene group.

In an exemplary embodiment of the present disclosure, R101 and R102, which are identical or different, are independently a substituted or unsubstituted aryl group.

In an exemplary embodiment of the present disclosure, R101 and R102, which are identical or different, are independently a substituted or unsubstituted monocyclic aryl group; or a substituted or unsubstituted polycyclic aryl group.

In an exemplary embodiment of the present disclosure, R101 and R102, which are identical or different, are independently a substituted or unsubstituted phenyl group; a substituted or unsubstituted biphenyl group; a substituted or unsubstituted terphenyl group; a substituted or unsubstituted naphthyl group; a substituted or unsubstituted anthracenyl group; a substituted or unsubstituted phenanthrenyl a substituted group; or unsubstituted triphenylene group; a substituted or unsubstituted pyrene group; or a substituted or unsubstituted fluorenyl group.

In an exemplary embodiment of the present disclosure, R101 and R102, which are identical or different, are independently a substituted or unsubstituted biphenyl group; or a substituted or unsubstituted fluorenyl group.

In an exemplary embodiment of the present disclosure, R101 and R102, which are identical or different, are independently a biphenyl group; or a fluorenyl group substituted with an alkyl group.

In an exemplary embodiment of the present disclosure, Chemical Formula HI-1 is represented by the following compounds.

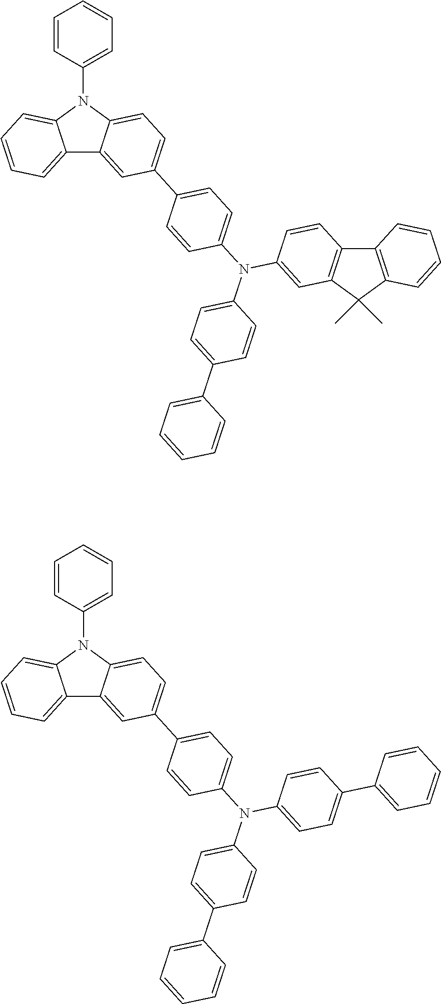 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccccc4)cc3)c3ccc(-c4ccc5c(c4)c4ccccc4n5-c4ccccc4)cc3)cc2)cc1]

In an exemplary embodiment of the present disclosure, the hole injection layer further contains a compound of Chemical Formula HI-2.

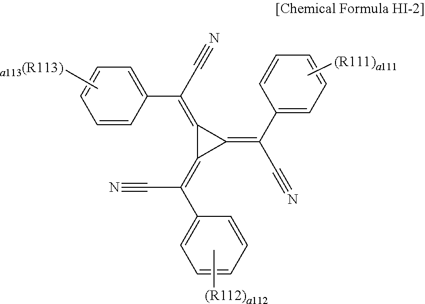

In Chemical Formula HI-2,
R111 to R113, which are identical or different, are independently hydrogen; a halogen group; a nitrile group; a substituted or unsubstituted alkyl group; a substituted or unsubstituted aryl group; or a substituted or unsubstituted heterocyclic group,
each of a111 to a113 is an integer from 1 to 5,
two or more R111s are identical or different if a111 is 2 or greater,
two or more R112s are identical or different if a112 is 2 or greater, and
two or more R113s are identical or different if a113 is 2 or greater.

In an exemplary embodiment of the present disclosure, R111 to R113, which are identical or different, are a halogen group; or a nitrile group.

In an exemplary embodiment of the present disclosure, R111 to R113, which are identical or different, are fluorine; or a nitrile group.

In an exemplary embodiment of the present disclosure, Chemical Formula HI-2 is represented by the following compound.

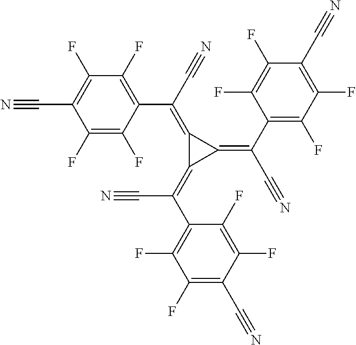 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

The hole transport layer can facilitate the transport of holes. As a hole transport material, a materials with high mobility for holes, which can receive holes from the anode or the hole injection layer and transfer them to the light-emitting layer, is suitable. Specific examples of the hole transport material include arylamine-based compounds, carbazole-based compounds, conductive polymers, block copolymers having both conjugated and non-conjugated segments, etc., although not being limited thereto.

In the organic light-emitting device of the present disclosure, a carbazole-based compound substituted with an arylamine group may be used in the hole transport layer, although not being limited thereto.

In the organic light-emitting device of the present disclosure, the hole transport layer contains the compound of Chemical Formula HI-1, which is described above.

An electron-blocking (hole control) layer may be provided between the hole transport layer and the light-emitting layer. The electronic blocking (hole control) layer is a layer which prevents electrons from flowing into the anode from the light-emitting layer and controls the flow of holes entering the light-emitting layer, thereby controlling the performance of the entire device. As an electronic blocking material (hole control material), a compound which has the ability to prevent the influx of electrons from the light-emitting layer to the anode and to control the flow of holes injected into the light-emitting layer or the light-emitting material, is preferred. In an exemplary embodiment, an arylamine-based organic material can be used in the electronic blocking (hole control) layer, although not being limited thereto.

In the organic light-emitting device of the present disclosure, the electron blocking (hole control) layer may contain an amine compound containing a carbazole group. For example, the compound may be one in which a carbazole group and an amine group are connected via an ortho-biphenylene group.

In the organic light-emitting device of the present disclosure, the electron blocking (hole control) layer may contain an amine compound containing phenanthrene. For example, it may be the following compound.

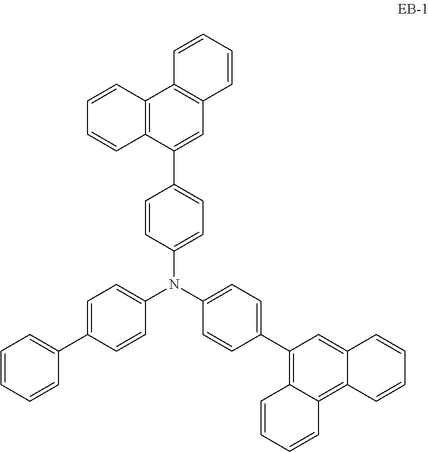 [SMILES: c1ccc(-c2ccc(N(c3ccc(-c4cc5ccccc5c5ccccc45)cc3)c3ccc(-c4cc5ccccc5c5ccccc45)cc3)cc2)cc1]

The light-emitting layer can emit red, green or blue light, and may be made of a phosphorescent material or a fluorescent material. As the light-emitting material, a material which can emit light in the visible light region by receiving holes and electrons from the hole transport layer and the electron transport layer, respectively, and combining them, and has good quantum efficiency for fluorescence or phosphorescence is preferred. Specific examples include 8-hydroxyquinoline aluminum complex (Alq₃); a carbazole-based compound; a dimerized styryl compound; BAlq; a 10-hydroxybenzoquinoline-metal a benzoxazole-, benzothiazole- or compound; benzimidazole-based compound; a poly(p-phenylene vinylene) (PPV)-based polymer; a spiro compound; polyfluorene, rubrene, etc., although not being limited thereto.

A host material of the light-emitting layer may be a condensed aromatic ring derivative, a heterocyclic compound, etc. Specifically, the condensed aromatic ring derivative may be an anthracene derivative, a pyrene derivative, a naphthalene derivative, a pentacene derivative, a phenanthrene compound, a fluoranthene compound, etc., and the heterocyclic compound may be a carbazole derivative, a dibenzofuran derivative, a ladder-type furan compound, a pyrimidine derivative, etc., although not being limited thereto.

In the organic light-emitting device of the present disclosure, the host may be a compound of Chemical Formula 1 or a carbazole derivative, although not being limited thereto.

In the organic light-emitting device of the present disclosure, the host may include the compound of Chemical Formula 1 as a second host, and a first host may be further included.

In the organic light-emitting device of the present disclosure, the first host may be a compound of Chemical Formula H.

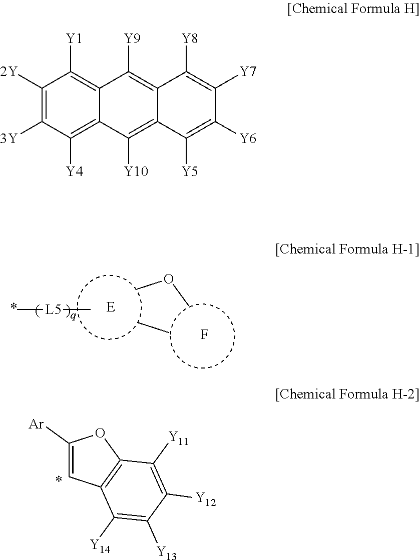

In Chemical Formula H,
one or more of Y1 to Y10 is bonded to the * site of Chemical Formulas H-1 or H-2, and the remainder of Y1 to Y10 not bonded to Chemical Formula H-1 or H-2, and Y11 to Y14, which are identical or different, are independently hydrogen, deuterium, a substituted or unsubstituted aryl group, a substituted or unsubstituted cycloalkyl group, a substituted or unsubstituted heteroaryl group, or a substituted or unsubstituted silyl group,
E and F, which are identical or different, are independently a substituted or unsubstituted aromatic hydrocarbon ring; or a substituted or unsubstituted aromatic heterocycle,
L5 is a direct bond, a substituted or unsubstituted arylene group, or a substituted or unsubstituted heteroarylene group,
q is an integer from 1 to 3, and
Ar is a substituted or unsubstituted aryl group, or a substituted or unsubstituted heteroaryl group.

In the organic light-emitting device of the present disclosure, the light-emitting layer may include a first light-emitting layer provided between the anode and the cathode; and a second light-emitting layer provided between the first light-emitting layer and the cathode, and in contact with the first light-emitting layer, wherein the first light-emitting layer may contain the compound of Chemical Formula 1, and the second light-emitting layer may contain the compound of Chemical Formula H.

In another organic light-emitting device of the present disclosure, the light-emitting layer may further include a third light-emitting layer provided between the second light-emitting layer and the cathode, and in contact with the second light-emitting layer, wherein the third light-emitting layer may contain the compound of Chemical Formula 1. Specifically, the light-emitting layer may include a first light-emitting layer provided between the anode and the cathode; a second light-emitting layer provided between the first light-emitting layer and the cathode and in contact with the first light-emitting layer; and a third light-emitting layer provided between the second light-emitting layer and the cathode and in contact with the second light-emitting layer, wherein the first light-emitting layer may contain the compound of Chemical Formula 1, the second light-emitting layer may contain the compound of Chemical Formula H, and the third light-emitting layer may contain the compound of Chemical Formula 1.

In the organic light-emitting device of the present disclosure, the compound of Chemical Formula H is any one of the following structures.

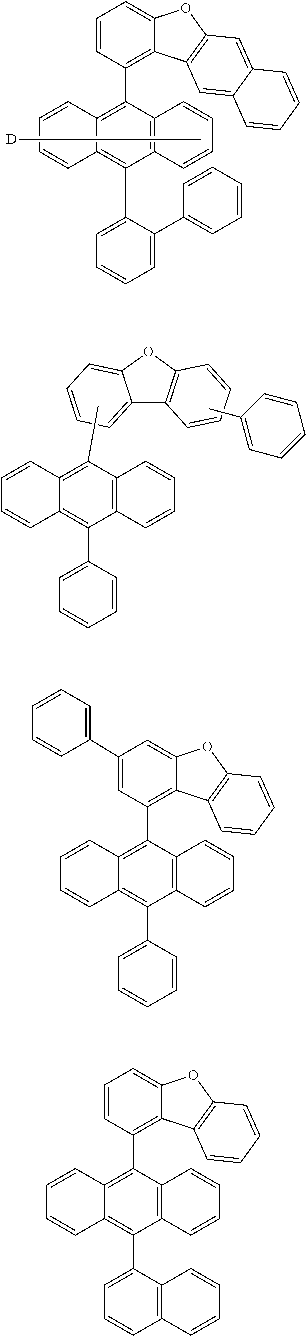

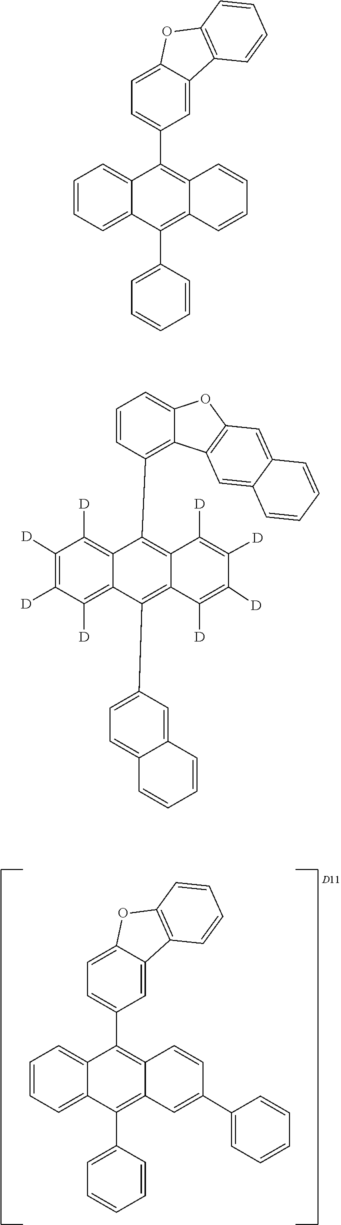

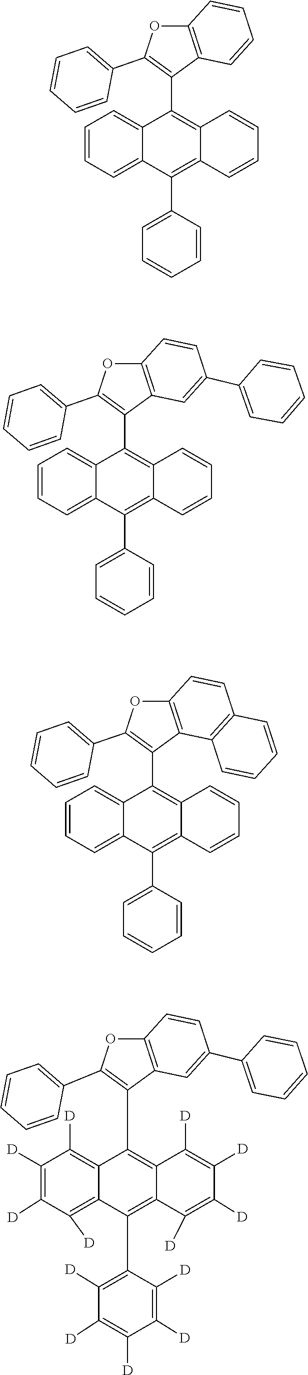 [SMILES: c1ccc(-c2oc3ccccc3c2-c2c3ccccc3c(-c3ccccc3)c3ccccc23)cc1 ; c1ccc(-c2ccc3oc(-c4ccccc4)c(-c4c5ccccc5c(-c5ccccc5)c5ccccc45)c3c2)cc1 ; c1ccc(-c2oc3ccc4ccccc4c3c2-c2c3ccccc3c(-c3ccccc3)c3ccccc23)cc1 ; [2H]c1c([2H])c([2H])c(-c2c3c([2H])c([2H])c([2H])c([2H])c3c(-c3c(-c4ccccc4)oc4ccc(-c5ccccc5)cc34)c3c([2H])c([2H])c([2H])c([2H])c23)c([2H])c1[2H]]

In the case where the light-emitting layer emits red light, a phosphorescent material such as PIQIr(acac) (bis(1-phenylisoquinoline) acetylacetonate iridium), PQIr (acac) (bis(1-phenylquinoline) acetylacetonate iridium), PQIr (tris(1-phenylquinoline) iridium) and PtOEP (octaethylporphyrin platinum) and a fluorescent material such as Alq₃ (tris(8-hydroxyquinolino)aluminum) may be used as a light-emitting dopant, although not being limited thereto. In the case where the light-emitting layer emits green light, a phosphorescent material such as Ir(ppy)₃ (tris(2-phenylpyridine) iridium) or a fluorescent material such as Alq₃ (tris(8-hydroxyquinolino)aluminum) may be used as a light-emitting dopant, although not being limited thereto. In the case where the light-emitting layer emits blue light, a phosphorescent material such as (4,6-F2ppy)₂Irpic or a fluorescent material such as spiro-DPVBi, spiro-6P, distyrylbenzene (DSB), distyrylarylene (DSA), a PFO-based polymer, and a PPV-based polymer may be used as a light-emitting dopant, although not being limited thereto.

In the organic light-emitting device of the present disclosure, the light-emitting dopant may include a boron-containing polycyclic compound, a Pt-containing compound, or an Ir complex, although not being limited thereto.

In the organic light-emitting device of the present disclosure, the light-emitting layer may contain the compound of Chemical Formula 1 as a host and may further contain a dopant.

In the organic light-emitting device of the present disclosure, the dopant may be a compound of Chemical Formula D or E.

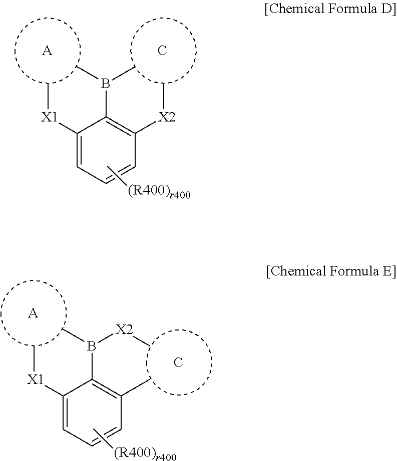

In Chemical Formulas D and E,
X1 and X2, which are identical or different, are independently CR′; or NR″, and at least one of X1 and X2 is NR″,
A and C, which are identical or different, are independently a substituted or unsubstituted hydrocarbon ring; or a substituted or unsubstituted heterocycle,
R400, R′ and R″, which are identical or different, are independently hydrogen; deuterium; a halogen group; a nitrile group; a substituted or unsubstituted alkyl group; a substituted or unsubstituted alkenyl group; a substituted or unsubstituted cycloalkyl group; a substituted or unsubstituted alkoxy group; a substituted or unsubstituted alkylthioxy group; a substituted or unsubstituted aryloxy group; a substituted or unsubstituted arylthioxy group; a substituted or unsubstituted silyl group; a substituted or unsubstituted amine group; a substituted or unsubstituted aryl group; or a substituted or unsubstituted heterocyclic group, or it is optionally bonded to an adjacent group to form a substituted or unsubstituted ring, and
r400 is an integer from 0 to 3, and the substituents in the parentheses are identical or different if r400 is 2 or greater.

In the organic light-emitting device of the present disclosure, A and C, which are identical or different, are independently a substituted or unsubstituted cycloalkane ring; a substituted or unsubstituted aromatic hydrocarbon ring; a substituted or unsubstituted heterocycle; or a combination thereof.

In the organic light-emitting device of the present disclosure, A and C, which are identical or different, are independently a substituted or unsubstituted 5-membered ring; a substituted or unsubstituted 6-membered ring; a substituted or unsubstituted polycyclic hydrocarbon ring; or a substituted or unsubstituted polycyclic heterocycle.

In the organic light-emitting device of the present disclosure, A and C, which are identical or different, are independently a substituted or unsubstituted benzene ring; a substituted or unsubstituted naphthalene ring; a substituted or unsubstituted tetrahydronaphthalene ring; a substituted or unsubstituted indene ring; a substituted or unsubstituted fluorene ring; a substituted or unsubstituted dibenzosilole ring; a substituted or unsubstituted dibenzofuran ring; or a substituted or unsubstituted dibenzothiophene ring.

In the organic light-emitting device of the present disclosure, A is a benzene ring substituted or unsubstituted with an alkyl group; or dibenzothiophene substituted or unsubstituted with an alkyl group.

In the organic light-emitting device of the present disclosure, A is a benzene ring substituted with a tert-butyl group; or dibenzothiophene substituted with a tert-butyl group.

In the organic light-emitting device of the present disclosure, C is a benzene ring substituted with an alkyl group; or a tetrahydronaphthalene ring substituted with an alkyl group.

In the organic light-emitting device of the present disclosure, C is a benzene ring substituted with a tert-butyl group; or a (1,1,4,4)-tetramethyl-(1, 2, 3, 4)-tetrahydronaphthalene ring.

In the organic light-emitting device of the present disclosure, the compound of Chemical Formula D or E has an adamantyl group or contains a ring of Structure 1.

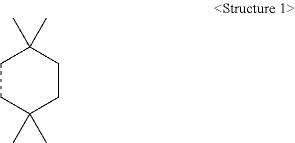

In the organic light-emitting device of the present disclosure, R400, R′ and R″, which are identical or different, are independently hydrogen; deuterium; a substituted or unsubstituted alkyl group; or a substituted or unsubstituted aryl group.

In the organic light-emitting device of the present disclosure, R400 is hydrogen; deuterium; or a C₁₋₁₀ alkyl group.

In the organic light-emitting device of the present disclosure, R400 is a methyl group; or a tert-butyl group.

In the organic light-emitting device of the present disclosure, R″ is a substituted or unsubstituted aryl group.

In the organic light-emitting device of the present disclosure, R″ is a substituted or unsubstituted phenyl group; or a substituted or unsubstituted tetrahydronaphthyl group.

In the organic light-emitting device of the present disclosure, R″ is a phenyl group substituted with a tert-butyl group; or a (1,1,4,4)-tetramethyl-(1, 2, 3, 4)-tetrahydronaphthyl group.

In the organic light-emitting device of the present disclosure, r400 is 1.

In the organic light-emitting device of the present disclosure, Chemical Formula D is Chemical Formula D-1 or Chemical Formula D-2.

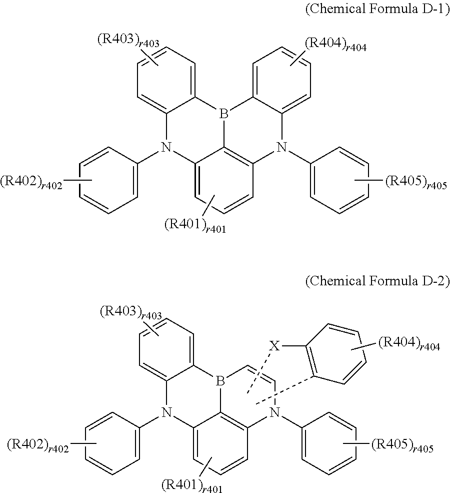

In Chemical Formulas D-1 and D-2,
X is O or S,
R401 to R405, which are identical or different, are independently hydrogen; deuterium; a halogen group; a nitrile group; a substituted or unsubstituted alkyl group; a substituted or unsubstituted alkenyl group; a substituted or unsubstituted cycloalkyl group; a substituted or unsubstituted alkoxy group; a substituted or unsubstituted alkylthioxy group; a substituted or unsubstituted aryloxy group; a substituted or unsubstituted arylthioxy group; a substituted or unsubstituted silyl group; a substituted or unsubstituted amine group; a substituted or unsubstituted aryl group; or a substituted or unsubstituted heterocyclic group, or it is optionally bonded to an adjacent group to form a substituted or unsubstituted cycloalkane ring; a substituted or unsubstituted aromatic hydrocarbon ring; or a substituted or unsubstituted heterocycle,
each of r401 to r405 is an integer from 0 to 3,
two or more R401s are identical or different if r401 is 2 or greater,
two or more R402s are identical or different if r402 is 2 or greater,
two or more R403s are identical or different if r403 is 2 or greater,
two or more R404s are identical or different if r404 is 2 or greater, and
two or more R405s are identical or different if r405 is 2 or greater.

In the organic light-emitting device of the present disclosure, Chemical Formula E is Chemical Formula E-1.

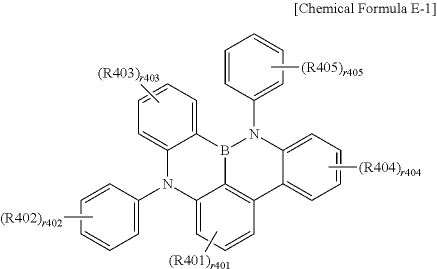

In Chemical Formula E-1,
R401 to R405, which are identical or different, are independently hydrogen; deuterium; a halogen group; a nitrile group; a substituted or unsubstituted alkyl group; a substituted or unsubstituted alkenyl group; a substituted or unsubstituted cycloalkyl group; a substituted or unsubstituted alkoxy group; a substituted or unsubstituted alkylthioxy group; a substituted or unsubstituted aryloxy group; a substituted or unsubstituted arylthioxy group; a substituted or unsubstituted silyl group; a substituted or unsubstituted amine group; a substituted or unsubstituted aryl group; or a substituted or unsubstituted heterocyclic group, or it is optionally bonded to an adjacent group to form a substituted or unsubstituted cycloalkane ring; a substituted or unsubstituted aromatic hydrocarbon ring; or a substituted or unsubstituted heterocycle,
each of r401 to r405 is an integer from 0 to 3,
two or more R401s are identical or different if r401 is 2 or greater,
two or more R402s are identical or different if r402 is 2 or greater,
two or more R403s are identical or different if r403 is 2 or greater,
two or more R404s are identical or different if r404 is 2 or greater, and
two or more R405s are identical or different if r405 is 2 or greater.

In the organic light-emitting device of the present disclosure, the compound of Chemical Formula D-1, D-2 or E-1 has an adamantyl group or contains a ring of Structure 1.

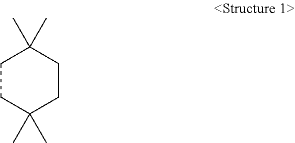

In the organic light-emitting device of the present disclosure, the dopant is any one of the following structures.

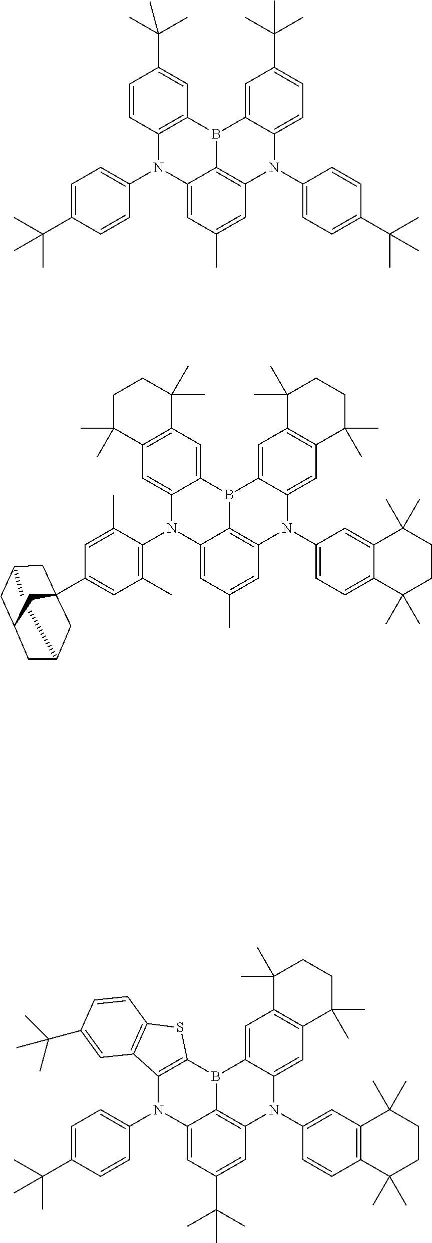

A hole blocking (electron control) layer may be provided between the electron transport layer and the light-emitting layer. The hole blocking (electron control) layer is a layer which blocks holes from flowing into the cathode from the light-emitting layer and controls the electrons flowing into the light-emitting layer, thereby controlling the performance of the entire device. As a hole blocking (electronic control) material, a compound having the ability to prevent the influx of holes from the light-emitting layer to the cathode and to control the electrons injected into the light-emitting layer or the light-emitting material, is preferred. As the hole blocking (electronic control) material, an appropriate material can be used depending on the composition of the organic layer used in the device. Specifically, an oxadiazole derivative, a triazole derivative, a phenanthroline derivative, BCP, an aluminum complex, etc. may be used, although not being limited thereto.

In an exemplary embodiment of the present disclosure, the hole blocking (electronic control) layer may contain a compound having a structure in which a nitrogen-containing ring is directly or indirectly connected to a spiro[fluorene-9,9′-xanthene] structure.

In an exemplary embodiment of the present disclosure, the electron control layer contains a compound of Chemical Formula HB-1.

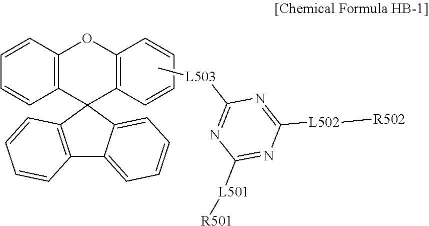

In Chemical Formula HB-1,
L501 to L503, which are identical or different, are independently a direct bond; or a substituted or unsubstituted arylene group, and
R501 and R502, which are identical or different, are independently hydrogen; deuterium; a substituted or unsubstituted alkyl group; a substituted or unsubstituted amine group; a substituted or unsubstituted aryl group; or substituted or unsubstituted heterocyclic group.

In an exemplary embodiment of the present disclosure, L501 to L503, which are identical or different, are independently a direct bond; a substituted or unsubstituted phenylene group; or a substituted or unsubstituted naphthylene group.

In an exemplary embodiment of the present disclosure, L501 to L503, which are identical or different, are independently a direct bond; or a substituted or unsubstituted phenylene group.

In an exemplary embodiment of the present disclosure, L501 to L503, which are identical or different, are independently a direct bond; or a phenylene group.

In an exemplary embodiment of the present disclosure, R501 and R502, which are identical or different, are independently a substituted or unsubstituted aryl group.

In an exemplary embodiment of the present disclosure, R501 and R502, which are identical or different, are independently a substituted or unsubstituted monocyclic aryl group; or a substituted or unsubstituted polycyclic aryl group.

In an exemplary embodiment of the present disclosure, R501 and R502, which are identical or different, are independently a substituted or unsubstituted phenyl group; a substituted or unsubstituted biphenyl group; a substituted or unsubstituted terphenyl group; a substituted or unsubstituted naphthyl group; a substituted or unsubstituted anthracenyl group; a substituted or unsubstituted phenanthrenyl group; a substituted or unsubstituted triphenylene group; a substituted or unsubstituted pyrene group; or a substituted or unsubstituted fluorenyl group.

In an exemplary embodiment of the present disclosure, R501 and R502, which are identical or different, are independently a substituted or unsubstituted phenyl group; or a substituted or unsubstituted naphthyl group.

In an exemplary embodiment of the present disclosure, R501 and R502, which are identical or different, are independently a phenyl group or a naphthyl group.

In an exemplary embodiment of the present disclosure, the compound of Chemical Formula HB-1 is the following compound.

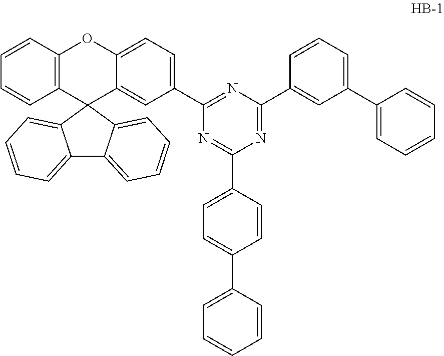 [SMILES: c1ccc(-c2ccc(-c3nc(-c4cccc(-c5ccccc5)c4)nc(-c4ccc5c(c4)C4(c6ccccc6O5)c5ccccc5-c5ccccc54)n3)cc2)cc1]

The electron transport layer may facilitate the transport of electrons. As an electron transport material, a material with high mobility for electrons, which can efficiently receive electrons injected from the cathode and transfer them to the light-emitting layer, is suitable. Specific examples include an Al complex of the aforementioned compound or 8-hydroxyquinoline; a complex including Alq3; an organic radical compound; a hydroxyflavone-metal complex, etc., although not being limited thereto.

In an exemplary embodiment of the present disclosure, the electron transport layer may contain a compound containing a nitrogen-containing ring, and may further contain an n-type dopant or an organometallic compound. According to an exemplary embodiment, the compound containing a nitrogen-containing ring may be a triazine-based compound, and the n-type dopant or organometallic compound may specifically be a lithium metal complex compound, e.g., 8-hydroxyquinolinatolithium, and the compound containing a nitrogen-containing ring and the n-type dopant (or organometallic compound) may be contained at a weight ratio of 2:8 to 8:2, for example, 4:6 to 6:4.

In an exemplary embodiment of the present disclosure, the electron transport layer contains a compound of Chemical Formula ET-1.

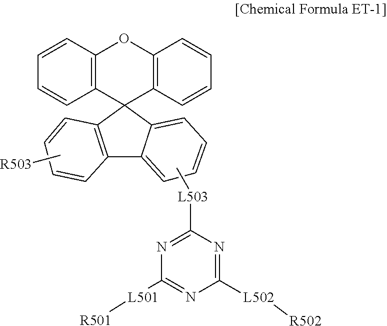

In Chemical Formula ET-1,
L501 to L503, which are identical or different, are independently a direct bond; or a substituted or unsubstituted arylene group, and
R501 and R502, which are identical or different, are independently hydrogen; deuterium; a substituted or unsubstituted alkyl group; a substituted or unsubstituted amine group; a substituted or unsubstituted aryl group; or a substituted or unsubstituted heterocyclic group, and
R503 is hydrogen; deuterium; a cyano group; or a substituted or unsubstituted alkyl group.

In an exemplary embodiment of the present disclosure, L501 to L503, which are identical or different, are independently a direct bond; a substituted or unsubstituted phenylene group; or a substituted or unsubstituted naphthylene group.

In an exemplary embodiment of the present disclosure, L501 to L503, which are identical or different, are independently a direct bond; or a substituted or unsubstituted phenylene group.

In an exemplary embodiment of the present disclosure, L501 to L503, which are identical or different, are independently a direct bond; or a phenylene group.

In an exemplary embodiment of the present disclosure, R501 and R502, which are identical or different, are independently a substituted or unsubstituted aryl group.

In an exemplary embodiment of the present disclosure, R501 and R502, which are identical or different, are independently a substituted or unsubstituted monocyclic aryl group; or a substituted or unsubstituted polycyclic aryl group.

In an exemplary embodiment of the present disclosure, R501 and R502, which are identical or different, are independently a substituted or unsubstituted phenyl group; a substituted or unsubstituted biphenyl group; a substituted or unsubstituted terphenyl group; a substituted or unsubstituted naphthyl group; a substituted or unsubstituted anthracenyl group; a substituted or unsubstituted phenanthrenyl group; a substituted or unsubstituted triphenylene group; a substituted or unsubstituted pyrene group; or a substituted or unsubstituted fluorenyl group.

In an exemplary embodiment of the present disclosure, R501 and R502, which are identical or different, are independently a substituted or unsubstituted phenyl group; or a substituted or unsubstituted naphthyl group.

In an exemplary embodiment of the present disclosure, R501 and R502, which are identical or different, are independently a phenyl group or a naphthyl group.

In an exemplary embodiment of the present disclosure, R503 is hydrogen; deuterium; or a cyano group.

In an exemplary embodiment of the present disclosure, R503 is a cyano group.

In an exemplary embodiment of the present disclosure, the compound of Chemical Formula ET-1 is the following compound.

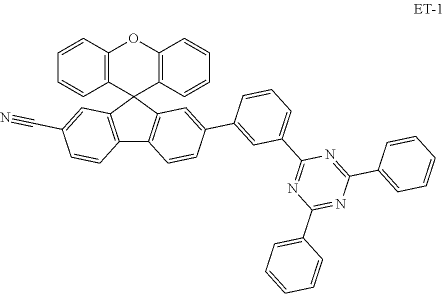 [SMILES: N#Cc1ccc2c(c1)C1(c3ccccc3Oc3ccccc31)c1cc(-c3cccc(-c4nc(-c5ccccc5)nc(-c5ccccc5)n4)c3)ccc1-2]

The electron injection layer may facilitate the injection of electrons. As an electron injection material, it has the ability to transport electrons, a compound with excellent thin film-forming ability, which has excellent electron injection effect from the cathode, has superior electron injection effect for the light-emitting layer or the light-emitting materials, prevents the movement of excitons generated in the light-emitting layer to the hole injection layer, is preferred. Specific examples include fluorenone, anthraquinodimethane, diphenoquinone, thiopyran dioxide, oxazole, oxadiazole, triazole, imidazole, perylenetetracarboxylic acid, fluorenylidene methane, anthrone, etc., and their derivatives, a metal complex compound, a nitrogen-containing five-membered ring derivative, etc., although not being limited thereto.

The metal complex compound includes 8-hydroxyquinolinatolithium, bis(8-hydroxyquinolinato) zinc, bis(8-hydroxyquinolinato) copper, bis(8-hydroxyquinolinato) manganese, tris(8-hydroxyquinolinato)aluminum, tris(2-methyl-8-hydroxyquinolinato)aluminum, tris(8-hydroxyquinolinato) gallium, bis(10-hydroxybenzo[h]quinolinato) beryllium, bis(10-hydroxybenzo[h]quinolinato) zinc, bis(2-methyl-8-quinolinato) chlorogallium, bis(2-methyl-8-quinolinate) (o-cresolato) gallium, bis(2-methyl-8-quinolinate) (1-naphtholato)aluminum, bis(2-methyl-8-quinolinate) (2-naphtholato) gallium, etc., although not being limited thereto.

In an exemplary embodiment of the present disclosure, the electron injection layer may contain lithium fluoride (LiF).

The organic light-emitting device according to the present disclosure may be of front emission type, rear emission type, or double-sided emission type depending on the material used.

Hereinafter, the present disclosure is described in detail through examples. However, the examples according to the present disclosure can be modified in various other forms, and the scope of this application should not be interpreted as being limited to the examples described below. The examples of the present disclosure are provided to more fully explain the present disclosure to those skilled in the art with an average level of knowledge.

### EXAMPLES

Hereinafter, the present specification will be described in more detail through examples. However, the following examples are only for illustrating the present disclosure and do not limit the present disclosure.

### EXAMPLES

### Synthesis Examples

### Synthesis Example 1

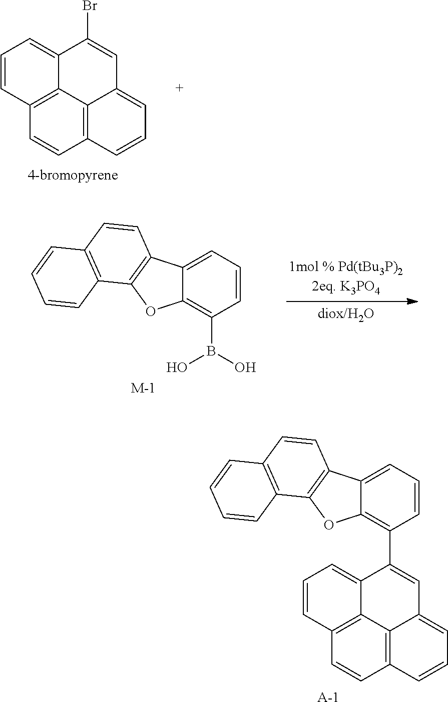

Under nitrogen atmosphere in a round-bottom flask, 4-bromopyrene (15 g, 53.35 mmol, leq.) and M-1 (15.4 g, 1.1 eq.) were dissolved in 300 mL of 1,4-dioxane and heated while stirring. Even if the compound was not completely dissolved, an aqueous solution of potassium phosphate (K₃PO₄) (22.7 g in 75 mL of H₂O, 2 eq.) was added. After adding bis(tri-tert-butylphosphine) palladium(0) (Pd(tBu₃P)₂) (0.3 g, 1 mol %), the mixture was stir heated for 4 hours while heating. After lowering temperature to room temperature and removing the aqueous layer, compound A-1 (14.1 g, 63%) was prepared by drying with anhydrous magnesium sulfate, concentrating under reduced pressure, and recrystallizing under toluene condition.

MS [M+H]⁺=419.

### Synthesis Example 2

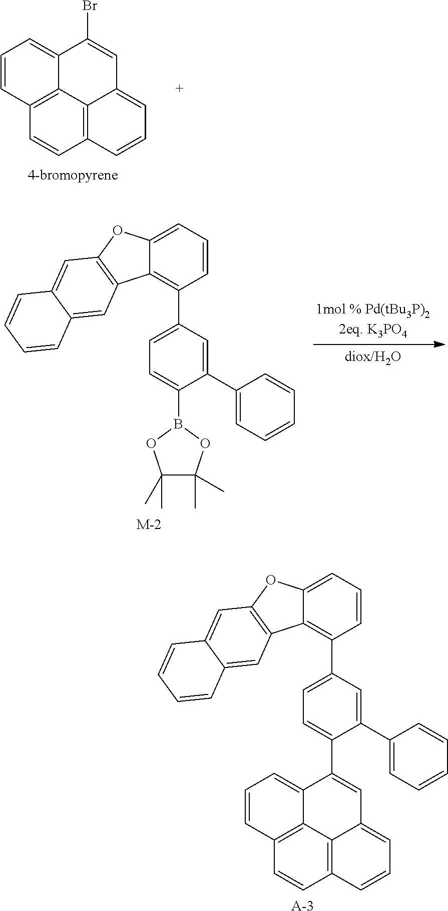

Compound A-3 was synthesized in the same manner as in Synthesis Example 1 using 4-bromopyrene (15 g, 1 eq.) and M-2 (1.1 eq.) as starting materials. Compound A-3 (15.5 g, 51%) was prepared by recrystallizing under toluene/ethyl acetate condition.

MS [M+H]⁺=571.

### Synthetic Example 3

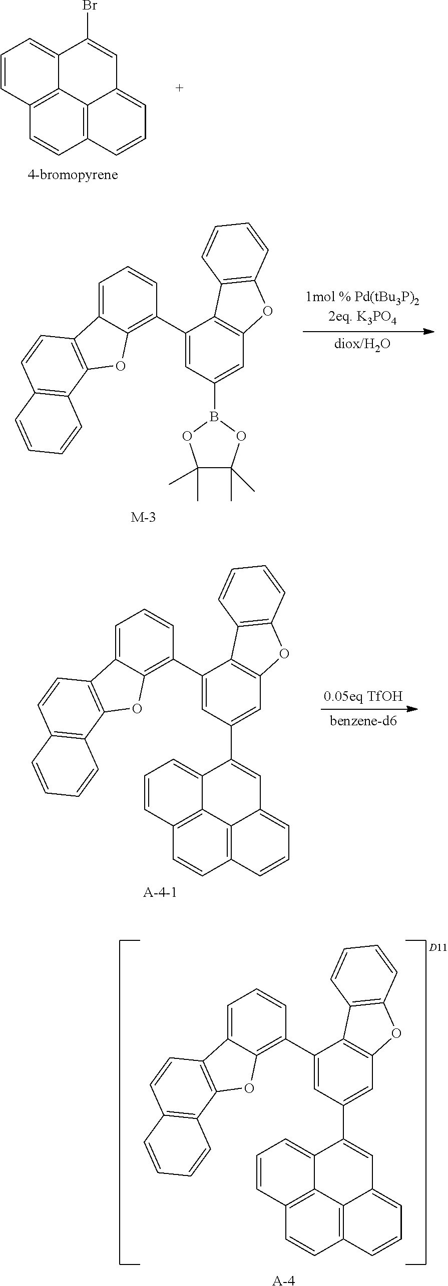

1) Compound A-4-1 was synthesized in the same manner as in Synthesis Example 1 using 4-bromopyrene (15 g, 1 eq.) and M-3 (1.1 eq.) as starting materials. Compound A-4-1 (16.8 g, 54%) was prepared by recrystallizing under toluene/ethyl acetate condition.

MS [M+H]⁺=585.

2) Compound A-4-1 (15 g, 25.6 mmol, 1 eq.) was dissolved in 300 mL of benzene-d6 while heating and stirring in a round-bottom flask under nitrogen atmosphere. When the compound was completely dissolved, trifluoromethanesulfonic acid (TfOH) (0.05 eq., 0.2 g) was added and the mixture was heated for 3 hours while stirring. After lowering temperature to room temperature and adding 50 mL of heavy water (D₂O), the mixture was stirred. After removing the aqueous layer, the residual acid was neutralized while slowly adding a saturated NaHCO₃ aqueous solution. The organic layer was collected, dried with anhydrous magnesium sulfate, concentrated under reduced pressure, and then recrystallized under toluene/ethanol and toluene conditions to prepare compound Å-4 (11 g, 72%).

MS [M+H]⁺=596.

### Synthesis Example 4

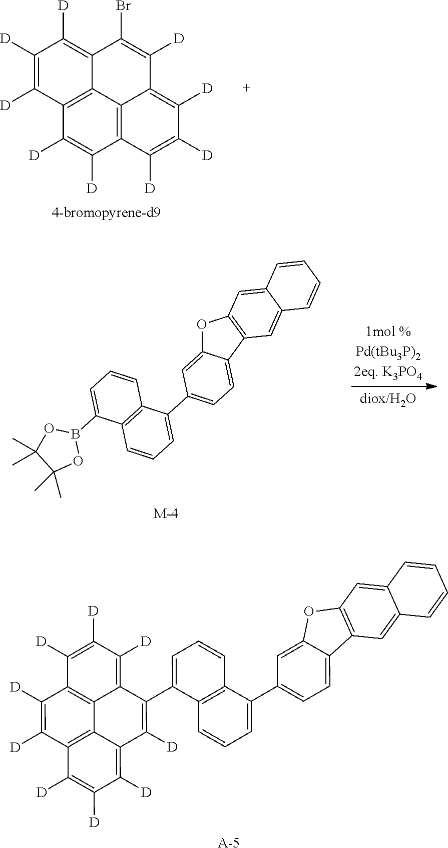 [SMILES: [2H]c1c([2H])c2c([2H])c([2H])c3c([2H])c([2H])c([2H])c4c(Br)c([2H])c(c1[2H])c2c34 ; CC1(C)OB(c2cccc3c(-c4ccc5c(c4)oc4cc6ccccc6cc45)cccc23)OC1(C)C ; "M-4" ; [2H]c1c([2H])c2c([2H])c([2H])c3c([2H])c([2H])c([2H])c4c(-c5cccc6c(-c7ccc8c(c7)oc7cc9ccccc9cc78)cccc56)c([2H])c(c1[2H])c2c34 ; "A-5"]

Compound A-5 was synthesized in the same manner as in Synthesis Example 1 using 4-bromopyrene-d9 (10 g, 1 eq.) and M-4 (1.1 eq.) as starting materials. Compound A-5 (14.9 g, 78%) was prepared by recrystallizing under toluene condition.

MS [M+H]⁺=554.

### Synthesis Example 5

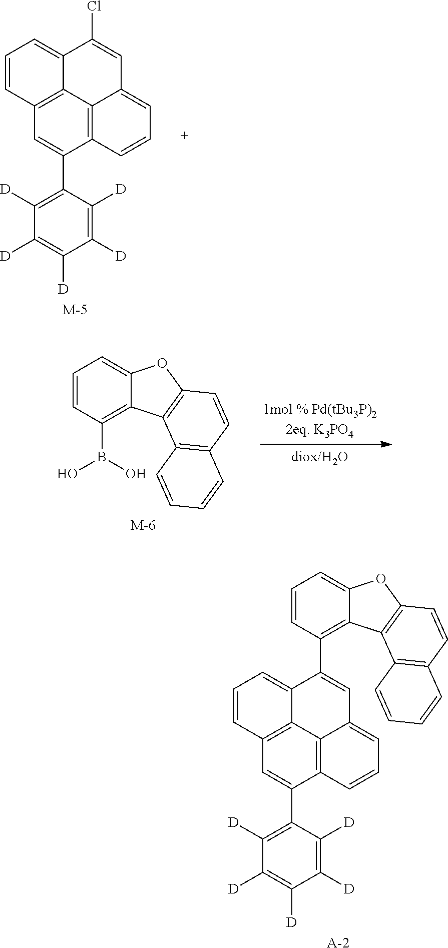

Compound A-2 was synthesized in the same manner as in Synthesis Example 1 using compound M-5 (10 g, 1 eq.) and M-6 (1.1 eq.) as starting materials. Compound A-2 (10.4 g, 66%) was prepared by recrystallizing under toluene/ethyl acetate condition.

MS [M+H]⁺=500.

### Synthesis Example 6

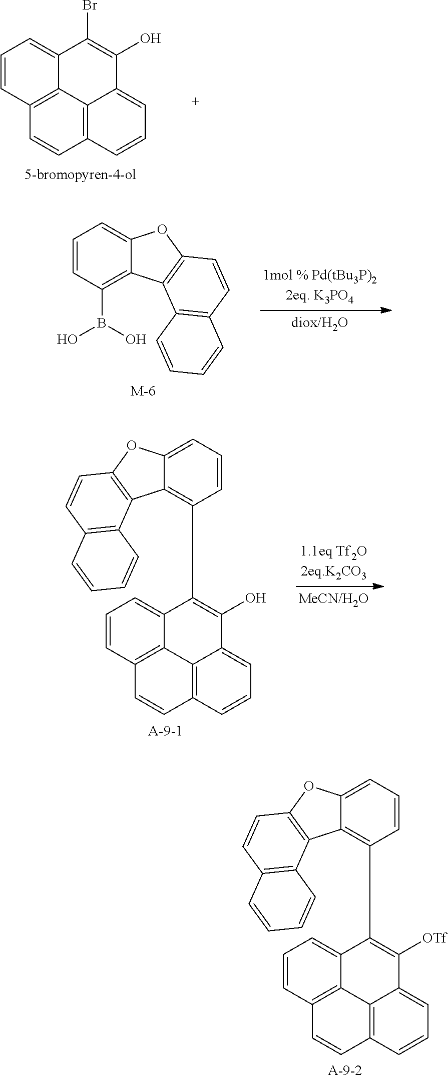

1) Compound A-9-1 was synthesized in the same manner as in Synthesis Example 1 using compound 5-bromopyren-4-ol (15 g, 1 eq.) and M-6 (1.1 eq) as starting materials. Compound A-9-1 (19.3 g, 88%) was prepared by recrystallizing under toluene/ethyl acetate condition.

MS [M+H]⁺=435.

2) Compound A-9-1 (19.3 g, 44.4 mmol, 1 eq.) and 400 mL of acetonitrile (MeCN) were added to a round-bottom flask and stirred under nitrogen atmosphere. After slowly adding a potassium carbonate aqueous solution, trifluoromethanesulfonic anhydride (Tf₂O) was added slowly. The mixture was stirred at room temperature for 4 hours. After adding water and ethanol and stirring further, the produced solid was filtered. The solid was dissolved in toluene and washed with water. After removing the aqueous layer and drying with anhydrous magnesium sulfate, compound Å-9-2 (20.9 g, 83%) was prepared by concentrating under reduced pressure and recrystallizing under toluene/ethanol condition.

### Synthesis Example 7

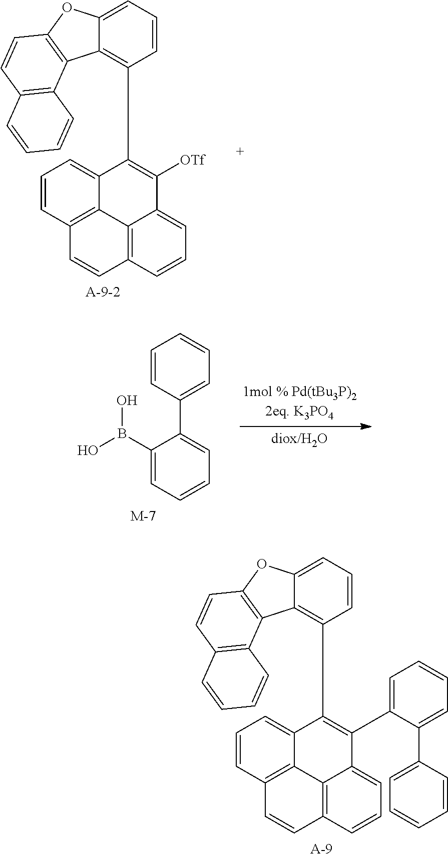

Compound A-9 was synthesized in the same manner as in Synthesis Example 1 using compound Å-9-2 (10 g, 1 eq.) and M-7 (1.1 eq.) as starting materials. Compound A-9 (4.8 g, 48%) was prepared by recrystallizing under toluene/ethyl acetate condition.

MS [M+H]⁺=571.

### Synthesis Example 8

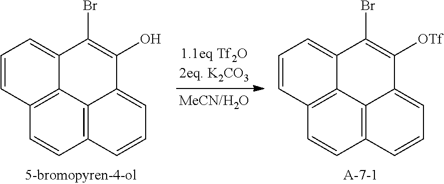 [SMILES: Oc1c(Br)c2cccc3ccc4cccc1c4c32 ; O=S(=O)(Oc1c(Br)c2cccc3ccc4cccc1c4c32)C(F)(F)F]

1) Compound A-7-1 was synthesized in the same manner as in Synthesis Example 6-2) using 5-bromopyren-4-ol (15 g, 1 eq.) as a starting material. Compound A-7-1 (17.8 g, 82%) was prepared by recrystallizing under toluene/ethanol condition.

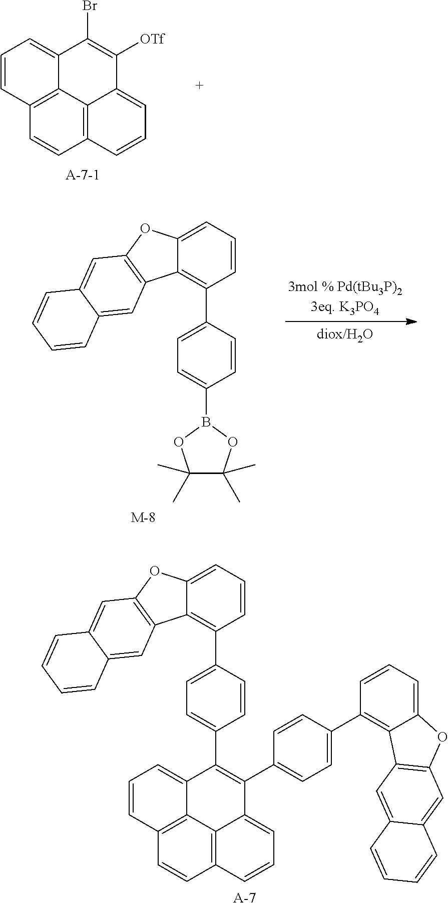

2) Compound A-7 was synthesized in the same manner as in Synthesis Example 1 using compound Å-7-1 (9 g, 1 eq.) and M-8 (2.1 eq.) as starting materials. Compound A-7 (7.3 g, 44%) was prepared by recrystallizing under toluene condition.

MS [M+H]⁺=787.

### Synthesis Example 9

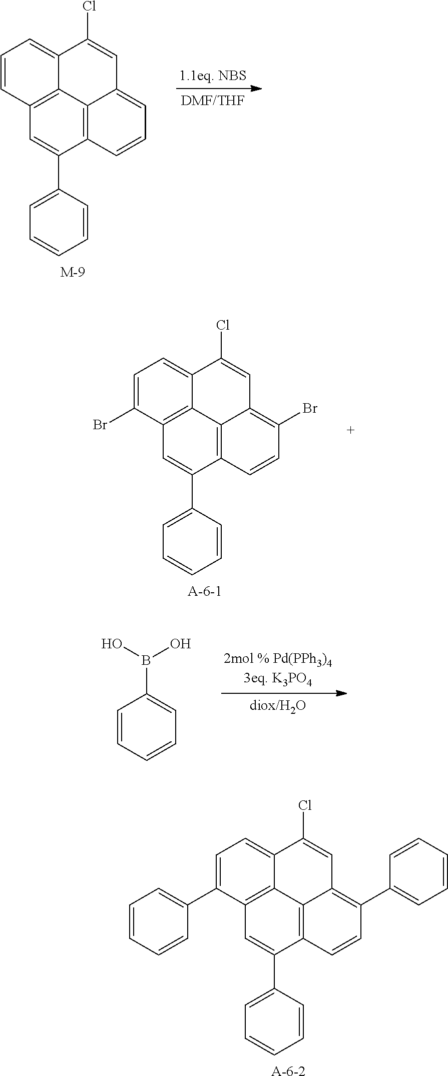

1) Compound M-9 (10 g, 31.9 mmol, 1 eq.) was dissolved in 150/50 mL of dimethylformamide (DMF)/tetrahydrofuran (THF) in a round-bottom flask under nitrogen atmosphere while stirring. After adding N-bromosuccinimide (NBS) (11.9 g, 2.1 eq.) at room temperature, the mixture was stirred at 60° C. for 10 hours. After the reaction was completed, the temperature was lowered to room temperature, and 100 ml of water and 400 mL of ethanol were added and the mixture was stirred for 30 minutes. After filtering, the produced solid was dissolved in toluene and washed with aq. Na₂S₂O₃ and aq. NaHCO₃. Then, the aqueous layer was removed. The organic layer was dried with anhydrous magnesium sulfate. Then, compound Å-6-1 (12 g, 80%) was prepared by concentrating under reduced pressure and recrystallizing under toluene condition.

MS [M+H]⁺=471.

2) Compound A-6-2 was synthesized in the same manner as in Synthesis Example 1 using compound Å-6-1 (12 g, 1 eq.) and phenylboronic acid (2.1 eq., 6.5 g) as starting materials and using tetrakis(triphenylphosphine)palladium(0) (Pd(PPh₃)₄) instead of bis(tri-tert-butylphosphine)palladium(0). Compound A-6-2 (9.2 g, 77%) was prepared by recrystallizing under toluene condition.

MS [M+H]⁺=466.

### Synthesis Example 11

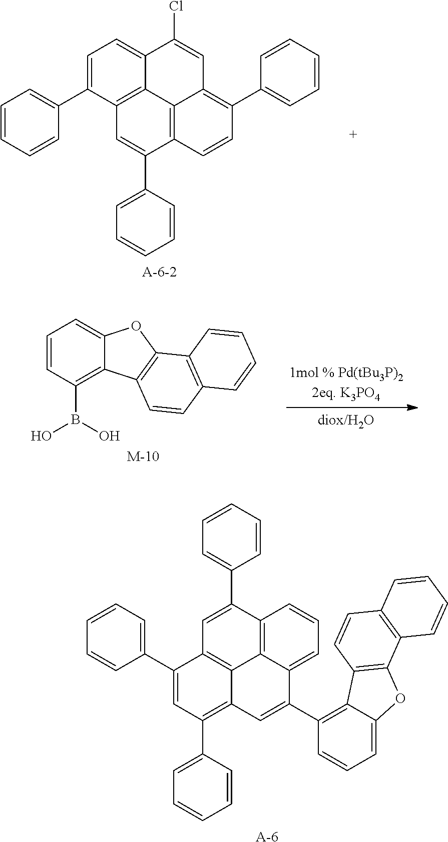

Compound A-6 was synthesized in the same manner as in Synthesis Example 1 using compound Å-6-2 (6 g, 1 eq.) and M-10 (1.1 eq.) as starting materials. Compound A-6 (5.6 g, 67%) was prepared by recrystallizing under toluene condition.

MS [M+H]⁺=648.

### Synthesis Example 12

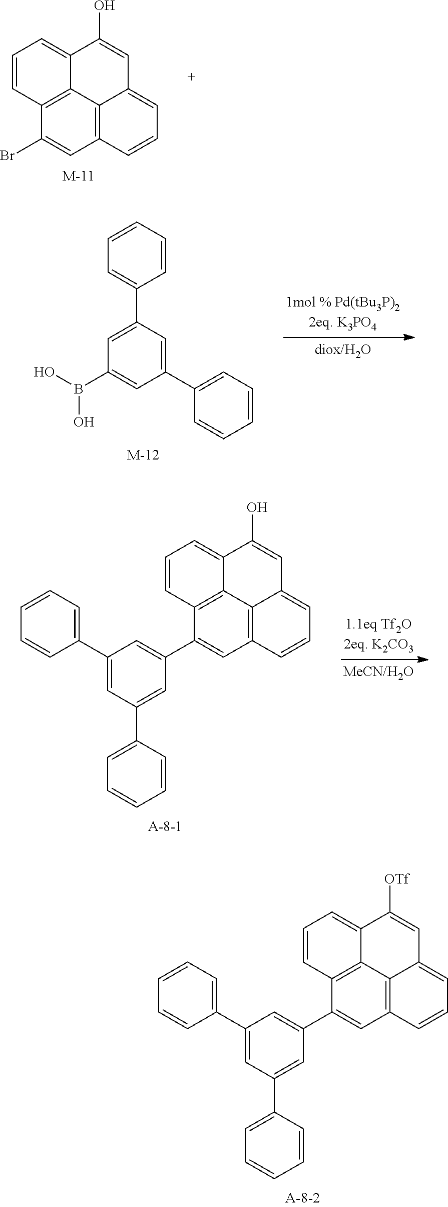

1) Compound A-8-1 was synthesized in the same manner as in Synthesis Example 1 using compound M-11 (15 g, 1 eq.) and M-12 (1.1 eq.) as starting materials. Compound A-8-1 (17.8 g, 79%) was prepared by recrystallizing under toluene condition.

MS [M+H]⁺=447.

2) Compound A-8-2 was synthesized in the same manner as in Synthesis Example 6-2) using compound Å-8-1 (17.8 g, 1 eq.) as a starting material. Compound A-8-2 (19.6 g, 85%) was prepared by recrystallizing under toluene/ethanol condition.

### Synthesis Example 13

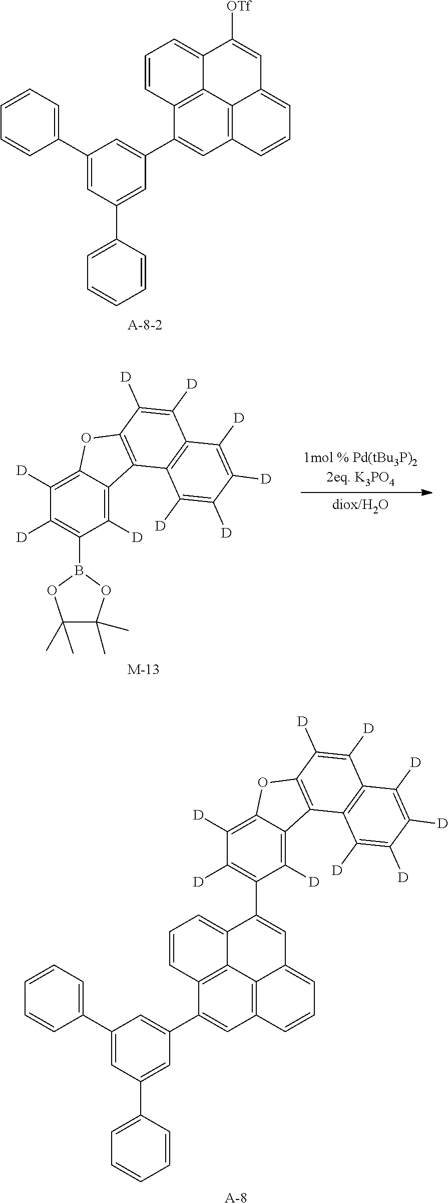

Compound A-8 was synthesized in the same manner as in Synthesis Example 1 using compound Å-8-2 (10 g, 1 eq.) and M-13 (1.1 eq.) as starting materials. Compound A-8 (5.1 g, 45%) was prepared by recrystallizing under toluene condition.

MS [M+H]⁺=655.

<Test Example 1> Confirmation of triplet depending on bonding position of pyrene

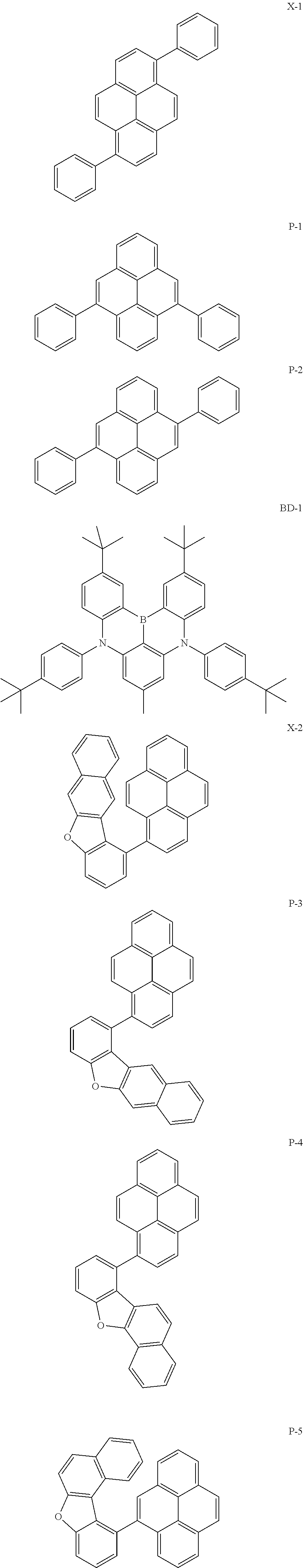

The energy levels of singlet and triplet states were calculated based on the HOMO and LUMO energy levels and absorption state of the compound using the TD-DFT (B3LYP) method and the 6-31G* basis method. The calculation result is as shown in Table 1 below.

TABLE 1

| | Comparative | Reference | Reference | Reference |
|---|---|---|---|---|
| | Example 1 | Example 1 | Example 2 | Example 3 |
| Compound | X-1 | P-1 | P-2 | BD-1 |
| HOMO (eV) | 5.15 | 5.23 | 5.22 | 4.57 |
| LUMO (eV) | 1.60 | 1.51 | 1.51 | 0.97 |
| S₁ (eV) | 3.36 | 3.57 | 3.55 | 3.07 |
| T₁ (eV) | 1.99 | 2.08 | 2.07 | 2.60 |
| | Comparative | | | |
| | Example 2 | Example 1 | Example 2 | Example 3 |
| Compound | X-2 | P-3 | P-4 | P-5 |
| HOMO (eV) | 5.36 | 5.39 | 5.35 | 5.33 |
| LUMO (eV) | 1.65 | 1.60 | 1.55 | 1.55 |
| S₁ (eV) | 3.35 | 3.41 | 3.50 | 3.41 |
| T₁ (eV) | 2.07 | 2.11 | 2.11 | 2.11 |

Looking at the experimental results when the functional group at the position 1 or 4 of pyrene was substituted and the positions 9 and 10 were further substituted, it can be seen that the triplet energy values are higher when substitutions were combined at the positions 4, 9 and 10. The triplet energy is lower than that of the blue dopant BD-1 used in blue light-emitting devices, but is sufficiently higher than that of an anthracene-based host material (T1,1.6-1.9 eV).

The higher triplet energy than that of the anthracene compound can reduce the triplet quenching of the dopant used together. In addition, the lower level than that of the dopant can prevent triplet exciton trapping by the dopant. As a result, the device's lifespan may be affected positively.

### <Test Example 2> Preparation of OLED

### Device Example 1-2

A substrate on which ITO/Ag/ITO were deposited to 70 Å/1000 Å/70 Å was cut to a size of 50 mm×50 mm×0.5 mm and ultrasonically cleaned with distilled water wherein a detergent was dissolved. The detergent used was the product of Fischer Co., and the distilled water was the distilled water filtered two times with a filter of Millipore Co. ITO was washed for 30 minutes, and then ultrasonically cleaned twice for 10 minutes with distilled water. After the washing with distilled water, the ITO was ultrasonically cleansed using isopropyl alcohol, acetone and methanol sequentially, and then dried.

A hole injection layer was formed on the anode by thermally vacuum-depositing HT-1 and HI-1 at a ratio of 9:1 to a thickness of 80 Å. A hole transport layer was formed thereon by vacuum-depositing a hole transport material, HT-1, to a thickness of 1000 Å, and then a hole control layer was formed using the compound EB-1 (50 Å).

Then, after depositing a first compound and a second compound, which satisfy the condition of Table 2, through co-evaporation at a specific weight ratio (4:6 wt %) to a thickness of 300 Å, 3 weight parts of the blue fluorescent dopant BD-1 of was co-deposited based on 100 weight parts of the first compound and the second compound to form a light-emitting layer. Then, after forming an electron control layer by depositing HB-1 to a thickness of 50 Å, an electron transport layer with a thickness of 280 Å was formed from a 7:3 mixture of the compounds ET-1 and Liq.

After forming a cathode with magnesium and silver (1:4) to a thickness of 150 Å, aluminum was deposited to 600 Å to complete a device. In the above process, the deposition rate of organic materials was maintained at 0.4 to 0.7 Å/sec, and the deposition rate of aluminum was maintained at 2 Å/sec. The degree of vacuum during the deposition was maintained at 2×10⁻⁷ to 5×10⁻⁶ torr.

The host used in the light-emitting layer is shown in Table 2 below. The operation voltage and luminous efficiency (EQE) of the organic light-emitting device prepared were measured at a current density of 10 mA/cm². The time at which the initial luminance was decreased to 95% (T95) was measured at a current density of 15 mA/cm² and was shown with respective to that of Comparative Example 3.

### Device Examples 1-3 to 1-5

Organic light-emitting devices were prepared in the same manner as in Device Example 1-2, except that the compounds listed in Table 2 were used instead of the first compound and the second compound of Device Example 1-2.

### Device Example 1-6

An organic light-emitting device was prepared in the same manner as in Device Example 1-2, except that the weight ratio of the first compound and the second compound described in Table 2 was changed instead of the first compound and the second compound of Device Example 1-2.

### Comparative Examples 4 to 6

Organic light-emitting devices were prepared in the same manner as in Device Example 1-2, except that the compounds listed in Table 2 were used instead of the first compound and the second compound of Device Example 1-2.

### Device Example 1-1 and Comparative Example 3

Organic light-emitting devices were prepared in the same manner as in Device Example 1-2, except that the single compounds listed in Table 2 were used instead of the first compound and the second compound of Device Example 1-2.

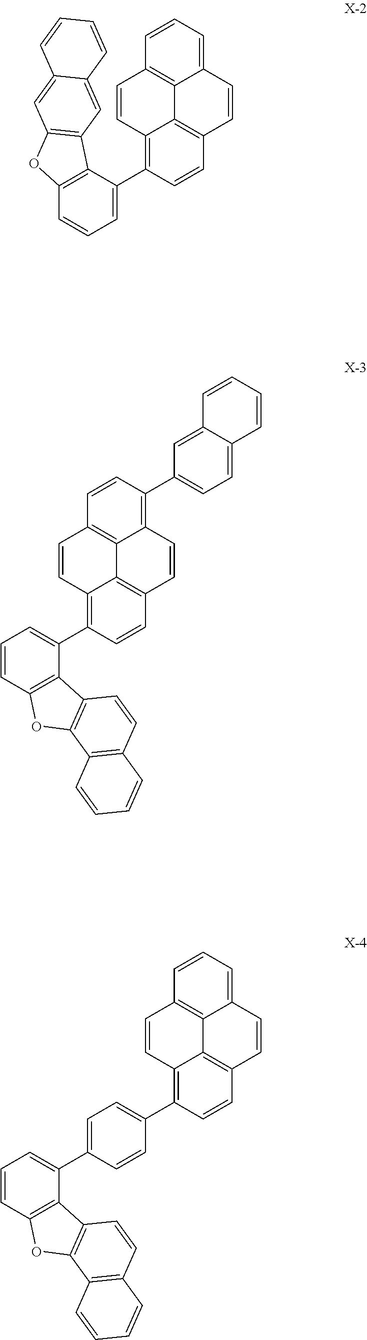 [SMILES: c1ccc2cc3c(cc2c1)oc1cccc(-c2ccc4ccc5cccc6ccc2c4c56)c13 ; c1ccc2cc(-c3ccc4ccc5c(-c6cccc7oc8c9ccccc9ccc8c67)ccc6ccc3c4c65)ccc2c1 ; c1ccc2c(c1)ccc1c2oc2cccc(-c3ccc(-c4ccc5ccc6cccc7ccc4c5c67)cc3)c21]

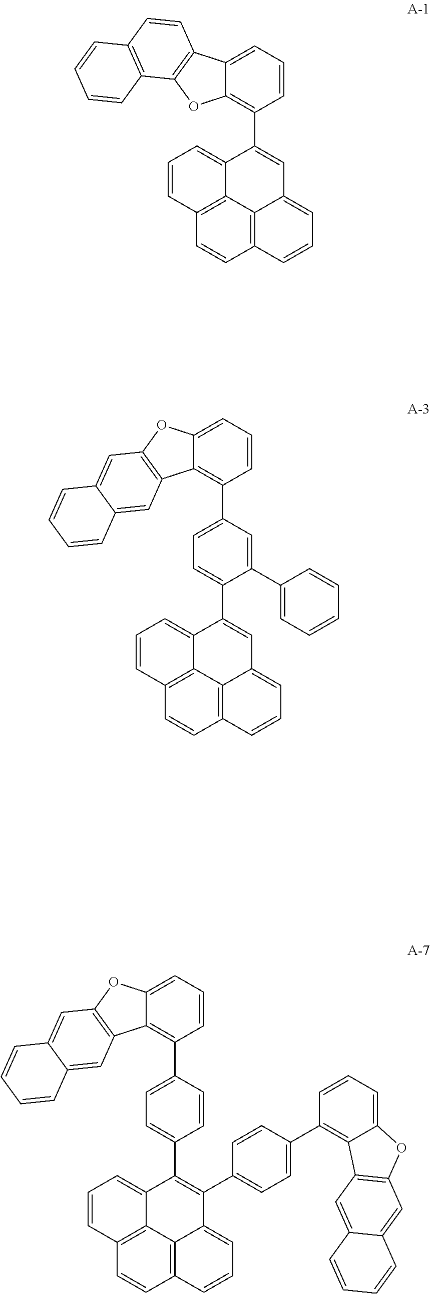 [SMILES: c1ccc2c(c1)ccc1c3cccc(-c4cc5cccc6ccc7cccc4c7c65)c3oc21 ; c1ccc(-c2cc(-c3cccc4oc5cc6ccccc6cc5c34)ccc2-c2cc3cccc4ccc5cccc2c5c43)cc1 ; c1ccc2cc3c(cc2c1)oc1cccc(-c2ccc(-c4c(-c5ccc(-c6cccc7oc8cc9ccccc9cc8c67)cc5)c5cccc6ccc7cccc4c7c65)cc2)c13]

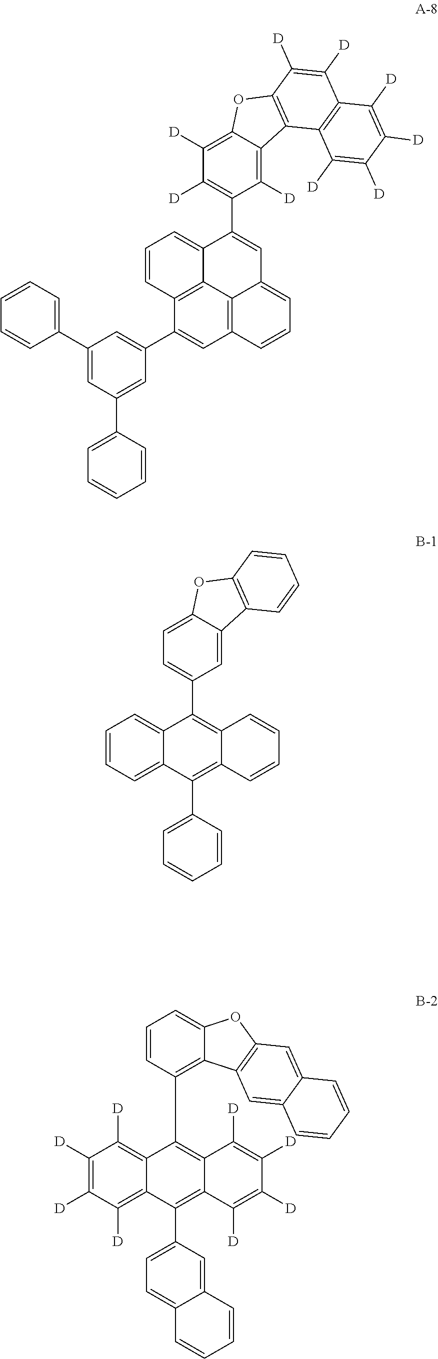 [SMILES: [2H]c1c(-c2cc3cccc4cc(-c5cc(-c6ccccc6)cc(-c6ccccc6)c5)c5cccc2c5c43)c([2H])c2c(oc3c([2H])c([2H])c4c([2H])c([2H])c([2H])c([2H])c4c32)c1[2H] ; c1ccc(-c2c3ccccc3c(-c3ccc4oc5ccccc5c4c3)c3ccccc23)cc1 ; [2H]c1c([2H])c([2H])c2c(-c3cccc4oc5cc6ccccc6cc5c34)c3c([2H])c([2H])c([2H])c([2H])c3c(-c3ccc4ccccc4c3)c2c1[2H]]

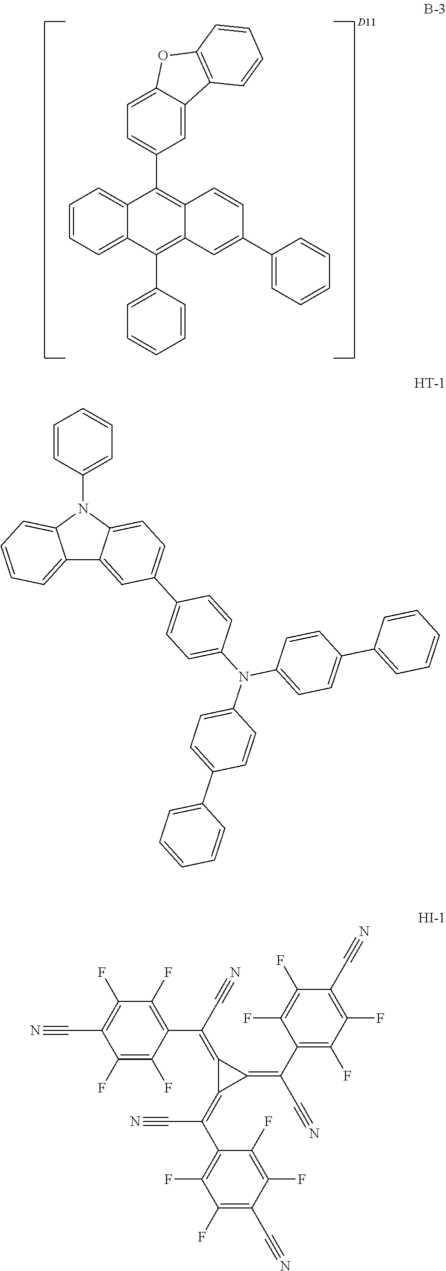

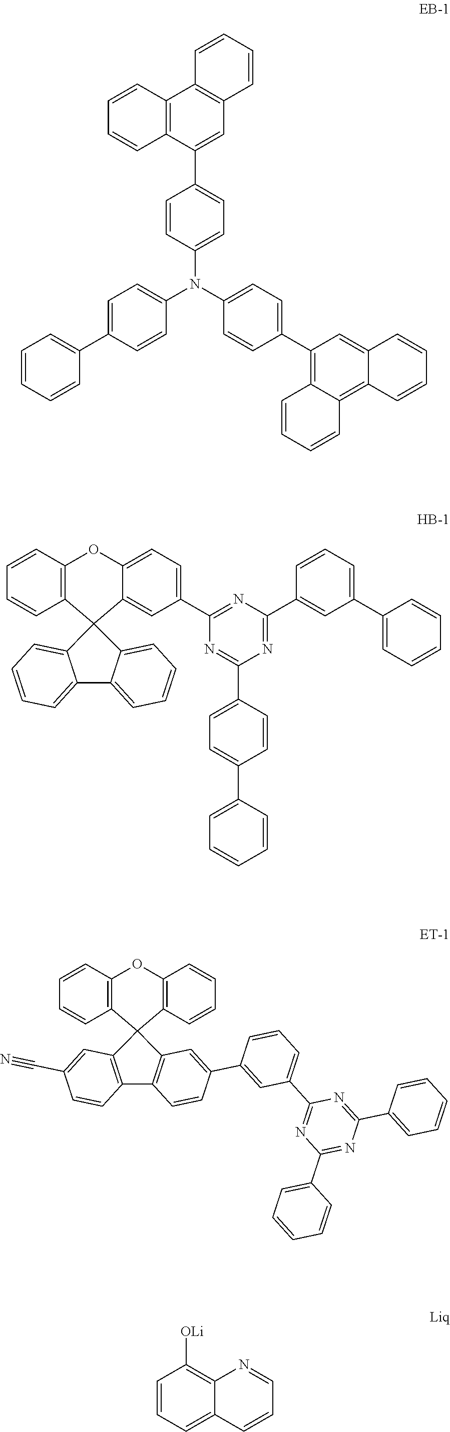

TABLE 2

| | | | 10 mA/cm² | | |
|---|---|---|---|---|---|
| | | | Opera- | | 15 |
| | Blue light-emitting layer | | tion | Effi- | mA/cm² |
| | First | Second | voltage | ciency | Lifespan |
| | compound | compound | (V) | (EQE, %) | ratio |
| Device | A-3 | — | 3.93 | 5.97 | 1.10 |
| Example 1-1 | | | | | |
| Device | A-1 | B-2 | 3.70 | 7.32 | 1.67 |
| Example 1-2 | | | | | |
| Device | A-3 | B-1 | 3.73 | 7.44 | 1.58 |
| Example 1-3 | | | | | |
| Device | A-7 | B-2 | 3.70 | 7.40 | 1.73 |
| Example 1-4 | | | | | |
| Device | A-8 | B-3 | 3.74 | 7.47 | 1.75 |
| Example 1-5 | | | | | |
| Device | A-3 | B-1 | 3.80 | 7.23 | 1.61 |
| Example 1-6 | 6:4, wt % | | | | |
| Comparative | X-2 | — | 3.89 | 5.82 | 1.00 |
| Example 3 | | | | | |
| Comparative | X-2 | B-1 | 3.69 | 7.09 | 1.45 |
| Example 4 | | | | | |
| Comparative | X-3 | B-1 | 3.72 | 7.16 | 1.36 |
| Example 5 | | | | | |
| Comparative | X-4 | B-2 | 3.75 | 7.19 | 1.36 |
| Example 6 | | | | | |

### Device Example 2

### <Device Example 2-1> Preparation of Organic Light-Emitting Device

A glass substrate on which indium tin oxide (ITO) was coated to a thickness of 1,000 Å was put in distilled water in which a detergent was dissolved and then cleansed ultrasonically. As the detergent, the product of Fischer Co. was used. And, as the distilled water, distilled water filtered twice with a filter available from Millipore Co. was used. After washing the ITO for 30 minutes, ultrasonic cleansing with distilled water was repeated twice for 10 minutes. After the cleansing with distilled water was completed, ultrasonic cleaning was performed using isopropyl alcohol, acetone and methanol as solvents, followed by drying and transporting to a plasma cleaner. Then, the substrate was cleaned for 5 minutes using oxygen plasma and then transported to a vacuum deposition system.

A hole injection layer was formed by thermally vacuum-depositing the compound of Chemical Formula HT-1 and the compound of Chemical Formula HI-1 on the prepared ITO transparent electrode at a ratio of 98:4 (molar ratio) to a thickness of 80 Å, and on top of that, the compound HT-1, which is a material for transporting holes, was vacuum-deposited to a thickness of 1150 Å to form a hole transport layer. Then, a hole control layer was formed using the compound EB-1 (80 Å). Then, a first light-emitting layer was formed by co-depositing the compound Å-1 (host) and the blue fluorescent dopant BD-1 at a weight ratio (wt %) of 97:3, and a second light-emitting layer was formed by co-depositing the compound B-2 (host) and the blue fluorescent dopant BD-1 at a weight ratio (wt %) of 97:3. The thickness of the first light-emitting layer and the second light-emitting layer is shown in Table 3 below, and the total thickness of the two light-emitting layers was 350 Å.

Then, an electron control layer was formed by depositing the compound HB-1 to a thickness of 50 Å, and an electron transport layer with a thickness of 300 Å was formed from a 7:3 mixture of the compounds ET-1 and Liq.

After forming a cathode with magnesium and silver (1:4) to a thickness of 150 Å, aluminum was deposited to 600 Å to complete a device. In the above process, the deposition rate of organic materials was maintained at 0.4 to 0.7 Å/sec, and the deposition rate of aluminum was maintained at 2 Å/sec. The degree of vacuum during the deposition was maintained at 2×10⁻⁷ to 5×10⁻⁶ torr.

The host used in the light-emitting layer is described in in Table 3. The operation voltage and luminous efficiency (EQE) of the organic light-emitting device prepared therefrom were measured at a current density of 10 mA/cm². The time at which the luminance was decreased to 95% of the initial luminance (T95) was measured at a current density of 15 mA/cm², and the result is shown as relative values with respect to Comparative Example 7.

### Device Examples 2-2 to 2-8

Organic light-emitting devices were prepared in the same manner as in Device Example 2-1, except that the compounds listed in Table 3 were used as a host and a dopant with the specified thicknesses, instead of those of the first and second light-emitting layers in Device Example 2-1.

### Comparative Example 7

An organic light-emitting device was prepared in the same manner as in Device Example 2-1, except that the single compound listed in Table 3 was used instead of those of the first and second light-emitting layers in Device Example 2-1. The weight ratio (wt %) of B-1 and BD-1 was the same at 97:3.

### Comparative Examples 8 to 11

Organic light-emitting devices were prepared in the same manner as in Device Example 2-1, except that the compounds listed in Table 3 were used as a host and a dopant with the specified thicknesses, instead of those of the first and second light-emitting layers in Device Example 2-1.

TABLE 3

| | | Blue light-emitting layer | | 10 mA/cm² | | 15 mA/cm² |
|---|---|---|---|---|---|---|
| | | First light- | Second light- | Operation | Efficiency | Lifespan, |
| | | emitting layer | emitting layer | voltage (V) | (EQE, %) | ratio |
| Device | Host | A-1, 140 Å | B-2, 210 Å | 3.75 | 7.79 | 1.35 |
| Example 2-1 | Dopant | BD-1 | BD-1 | | | |
| Device | Host | A-2, 140 Å | B-1, 210 Å | 3.73 | 7.95 | 1.28 |
| Example 2-2 | Dopant | BD-2 | BD-1 | | | |
| Device | Host | A-4, 140 Å | B-1, 210 Å | 3.78 | 7.65 | 1.33 |
| Example 2-3 | Dopant | BD-2 | BD-1 | | | |
| Device | Host | A-5, 140 Å | B-2, 210 Å | 3.75 | 7.58 | 1.30 |
| Example 2-4 | Dopant | BD-1 | BD-1 | | | |
| Device | Host | A-6, 140 Å | B-4, 210 Å | 3.80 | 7.96 | 1.44 |
| Example 2-5 | Dopant | BD-1 | BD-1 | | | |
| Device | Host | A-8, 140 Å | B-5, 210 Å | 3.71 | 7.88 | 1.41 |
| Example 2-6 | Dopant | BD-1 | BD-3 | | | |
| Device | Host | A-9, 140 Å | B-5, 210 Å | 3.72 | 7.69 | 1.31 |
| Example 2-7 | Dopant | BD-1 | BD-3 | | | |
| Device | Host | A-7, 175 Å | B-2, 175 Å | 3.69 | 7.64 | 1.29 |
| Example 2-8 | Dopant | BD-1 | BD-3 | | | |
| Comparative | Host | B-1, 350 Å | | 3.51 | 7.02 | 1.00 |
| Example 7 | Dopant | BD-1 | | | | |
| Comparative | Host | B-2, 140 Å | B-1, 210 Å | 3.41 | 7.10 | 1.08 |
| Example 8 | Dopant | BD-1 | BD-1 | | | |
| Comparative | Host | X-4, 140 Å | B-1, 210 Å | 3.71 | 7.59 | 1.22 |
| Example 9 | Dopant | BD-1 | BD-1 | | | |
| Comparative | Host | X-5, 140 Å | B-1, 210 Å | 3.54 | 6.25 | 0.67 |
| Example 10 | Dopant | BD-2 | BD-1 | | | |
| Comparative | Host | X-6, 175 Å | B-5, 175 Å | 3.32 | 5.69 | 0.24 |
| Example 11 | Dopant | BD-2 | BD-1 | | | |

The use of the compound of Chemical Formula 1 of the present disclosure, which has a T₁ higher than that of anthracene-based compounds, in the first light-emitting layer allowed the area where holes and electrons meet to form excitons (recombination zone) to be distinguished from the area where the excitons emit light (light-emitting zone). As a result, the efficiency of the device was improved and the lifespan was increased. As seen from the experimental results of Tables 2 and 3, the efficiency and lifespan performance of the devices varied slightly depending on the thickness ratio of the first host and the second host. And, it was confirmed that the better lifetime and efficiency performance were achieved when the first and second light-emitting layers were formed as a bilayer as in Device Example 2, as compared to when they were co-deposited as in Device Example 1.

1. A compound of Chemical Formula 1: 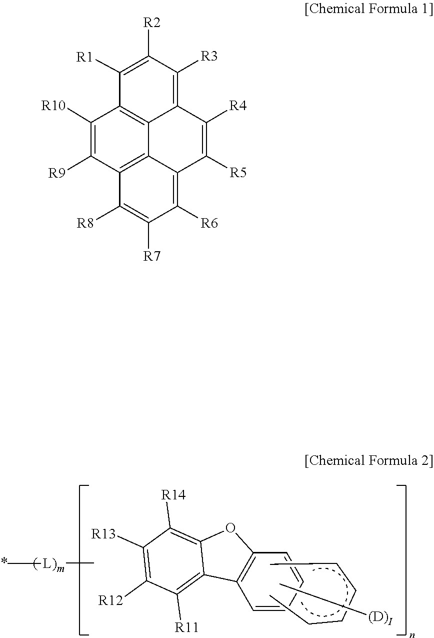
in Chemical Formulae 1 and 2,
one to four of R4, R5, R9 and R10 is independently connected to L of Chemical Formula 2, and L is connected to any one of R11 to R14 of Chemical Formula 2, the remainder of R4, R5, R9 and R10 not connected to L, R1 to R3, R6 to R8, and the remainder of R11 to R14 not connected to L, which are identical or different, are independently hydrogen, deuterium, a substituted or unsubstituted tetrahydronaphthalenyl group, a substituted or unsubstituted silyl group, or a substituted or unsubstituted aryl group,
L is a single bond, a substituted or unsubstituted arylene group, or a substituted or unsubstituted divalent heterocyclic group,
n is an integer from 1 to 4, and the structures within the parentheses are identical or different if n is 2 or greater,
m is an integer from 1 to 4, and two or more Ls are identical or different if m is 2 or greater,
1 is the number of deuterium, which is an integer from 0 to 6, and
* indicates the position of bonding.

2. The compound according to claim 1, wherein Chemical Formula 1 is any one of Chemical Formulas 1-1 to 1-4: 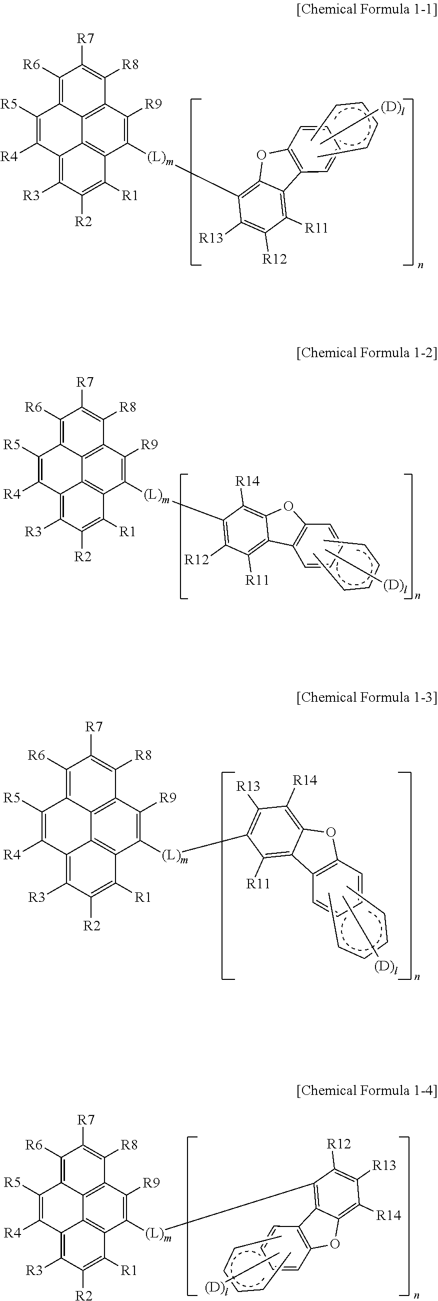
in Chemical Formulas 1-1 to 1-4,
L, l, m and n are the same as defined in Chemical Formula 2, and
R1 to R9 and R11 to R14, which are identical or different, are independently hydrogen, deuterium, a substituted or unsubstituted tetrahydronaphthalenyl group, a substituted or unsubstituted silyl group, or a substituted or unsubstituted aryl group.

3. The compound according to claim 1, wherein Chemical Formula 1 is any one of Chemical Formulas 1-A to 1-D: 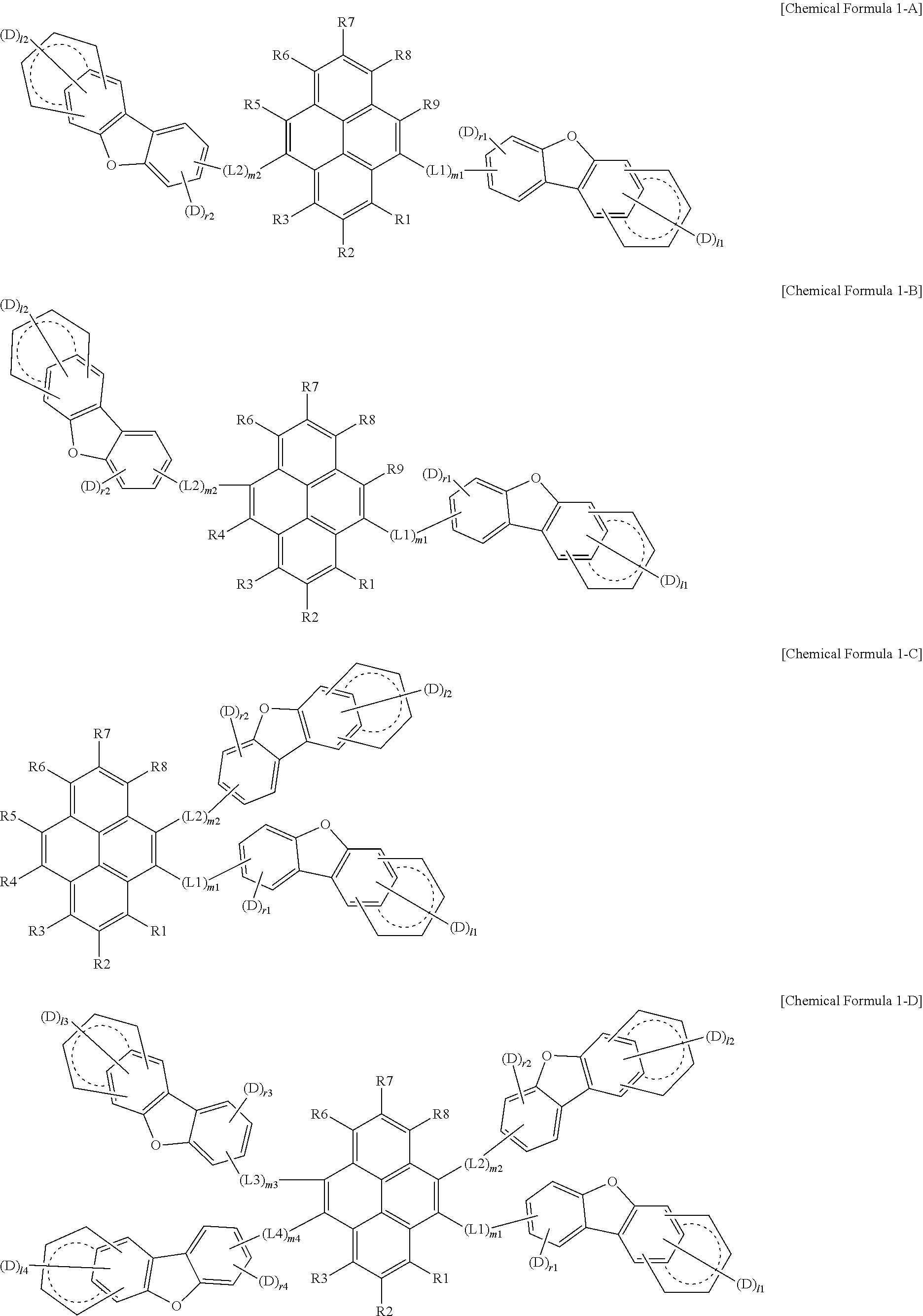
in Chemical Formulas 1-A to 1-D,
R1 to R9, which are identical or different, are independently hydrogen, deuterium, a substituted or unsubstituted tetrahydronaphthalenyl group, a substituted or unsubstituted silyl group, or a substituted or unsubstituted aryl group,
L1 to L4, which are identical or different, are independently a single bond, a substituted or unsubstituted arylene group, or a substituted or unsubstituted divalent heterocyclic group,
each of m1 to m4 is an integer from 1 to 4,
two or more L1s are identical or different if m1 is 2 or greater,
two or more L2s are identical or different if m2 is 2 or greater,
two or more L3s are identical or different if m3 is 2 or greater,
two or more L4s are identical or different if m4 is 2 or greater,
each of l1 to l4 is the number of deuterium, which is an integer from 0 to 6, and
each of r1 to r4 is the number of deuterium, which is an integer from 0 to 3.

4. The compound according to claim 1, wherein Chemical Formula 2 is any one of Chemical Formulas 2-1 to 2-3: 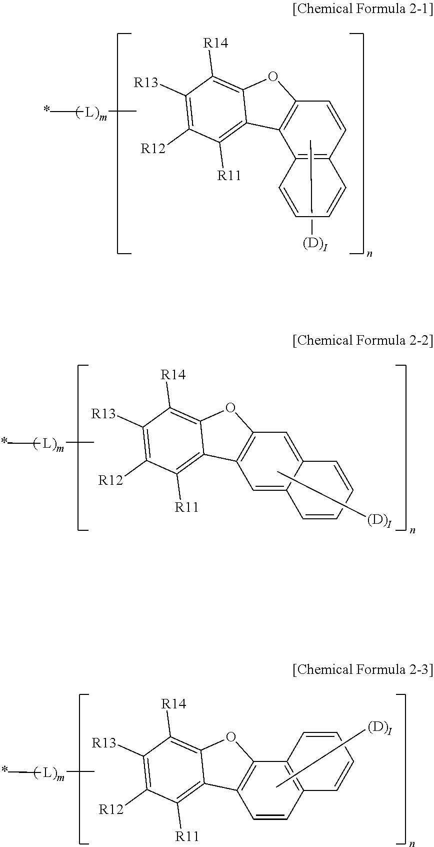
in Chemical Formulas 2-1 to 2-3,
l, m and n are the same as defined in Chemical Formula 2,
L is a single bond, a substituted or unsubstituted arylene group, or a substituted or unsubstituted divalent heterocyclic group,
L is connected to R11 or R14, and
each of the remainder of R11 or R14 not connected to L, R12 and R13 is hydrogen, deuterium, or a substituted or unsubstituted aryl group.

5. The compound according to claim 1, wherein L is a single bond, an arylene group substituted or unsubstituted with deuterium, or a divalent heterocyclic group substituted or unsubstituted with deuterium.

6. The compound according to claim 1, wherein n is 1.

7. The compound according to claim 1, wherein the compound of Chemical Formula 1 is one of the following structures:

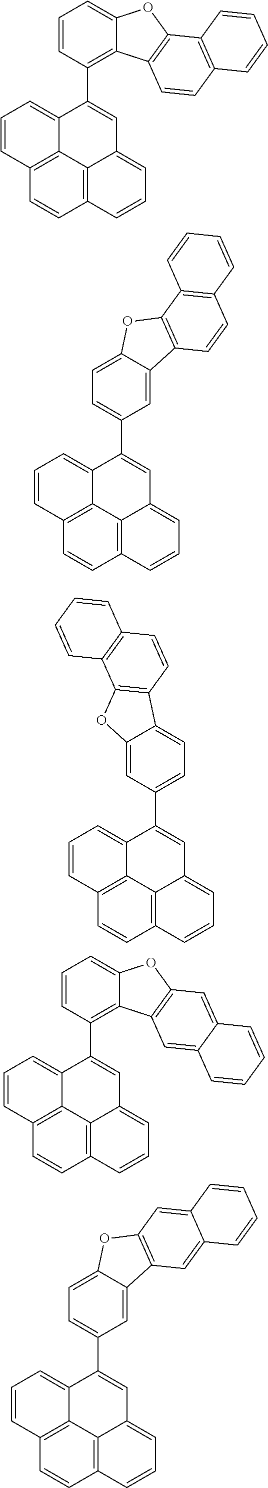

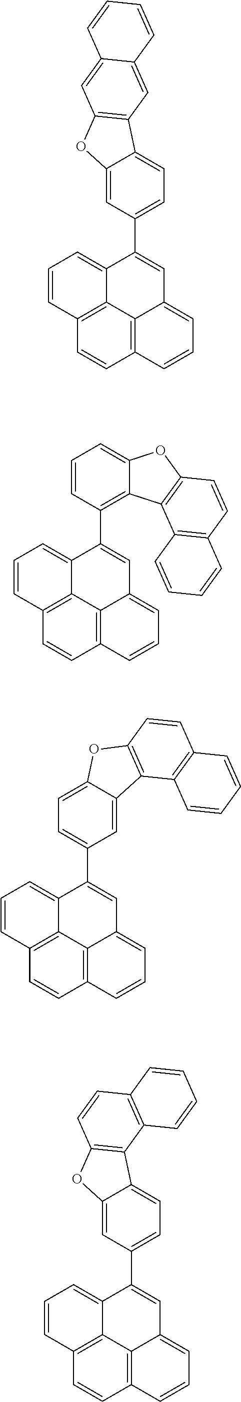 [SMILES: c1ccc2cc3c(cc2c1)oc1cc(-c2cc4cccc5ccc6cccc2c6c54)ccc13 ; c1ccc2c(c1)ccc1oc3cccc(-c4cc5cccc6ccc7cccc4c7c65)c3c12 ; c1ccc2c(c1)ccc1oc3ccc(-c4cc5cccc6ccc7cccc4c7c65)cc3c12 ; c1ccc2c(c1)ccc1oc3cc(-c4cc5cccc6ccc7cccc4c7c65)ccc3c12]

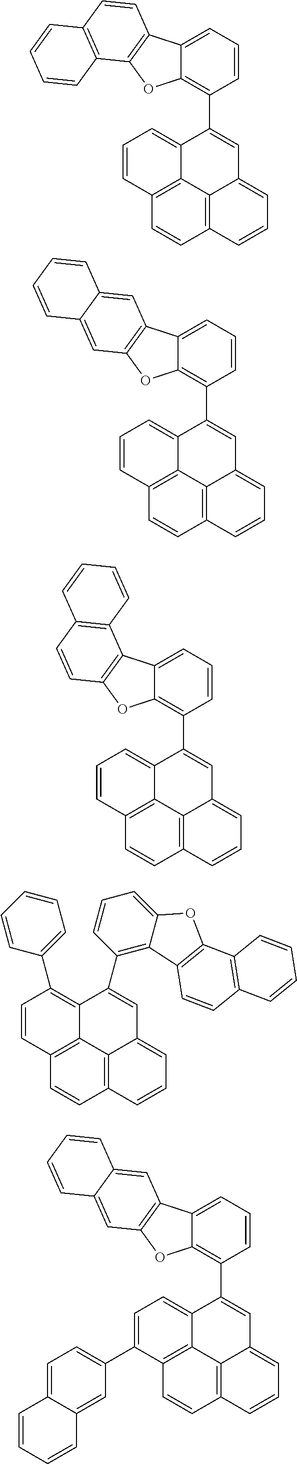

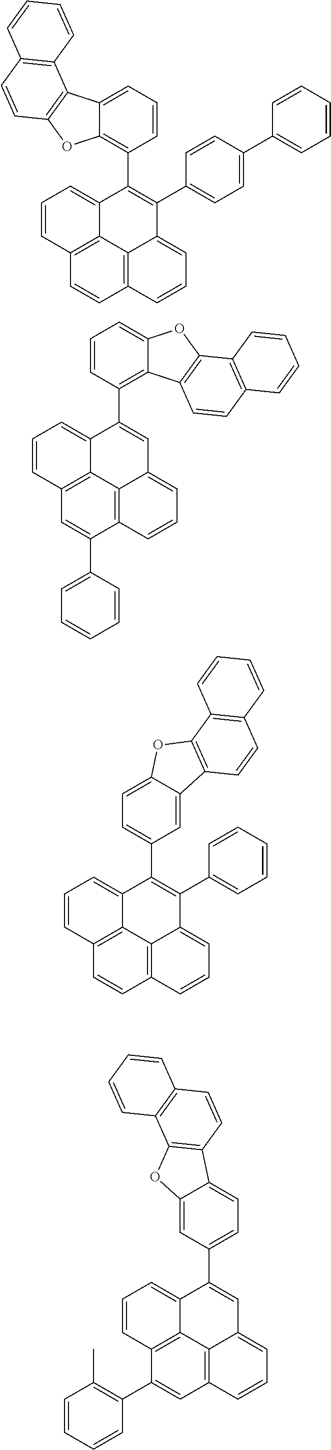

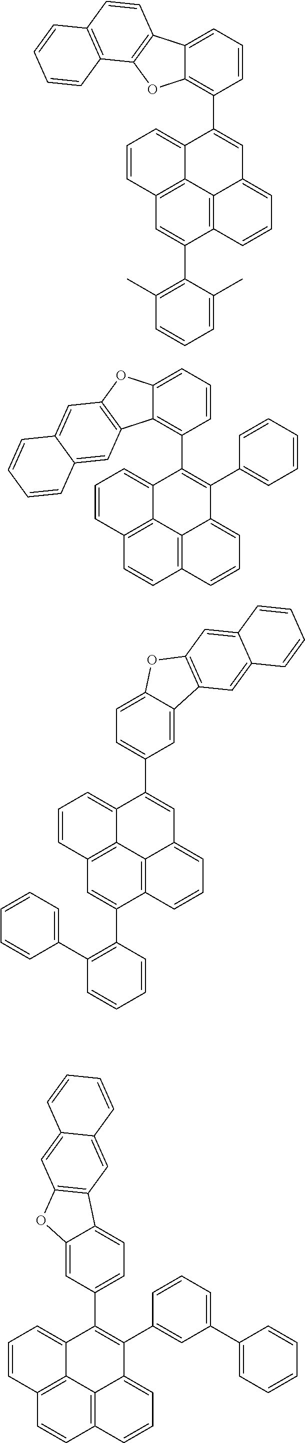

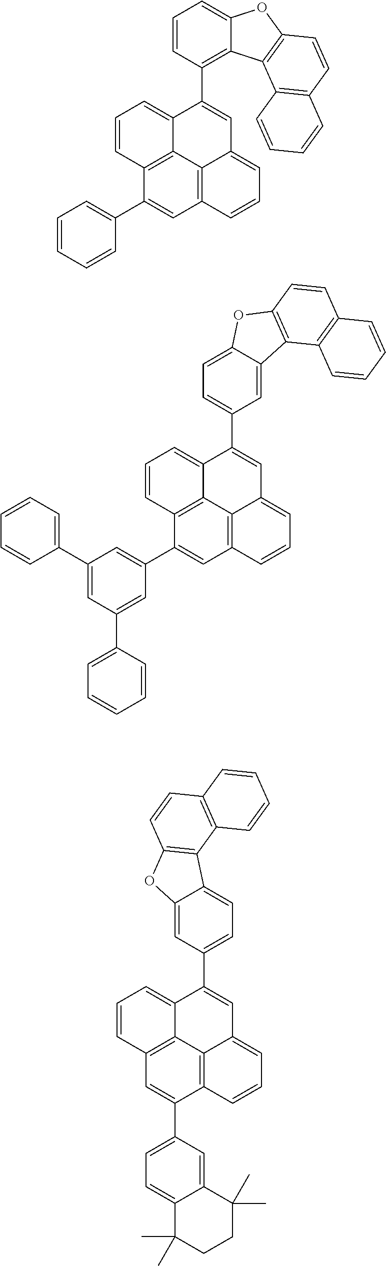 [SMILES: c1ccc(-c2cc3cccc4cc(-c5cccc6oc7ccc8ccccc8c7c56)c5cccc2c5c34)cc1 ; c1ccc(-c2cc(-c3ccccc3)cc(-c3cc4cccc5cc(-c6ccc7oc8ccc9ccccc9c8c7c6)c6cccc3c6c45)c2)cc1 ; CC1(C)CCC(C)(C)c2cc(-c3cc4cccc5c(-c6ccc7c(c6)oc6ccc8ccccc8c67)cc6cccc3c6c45)ccc21]

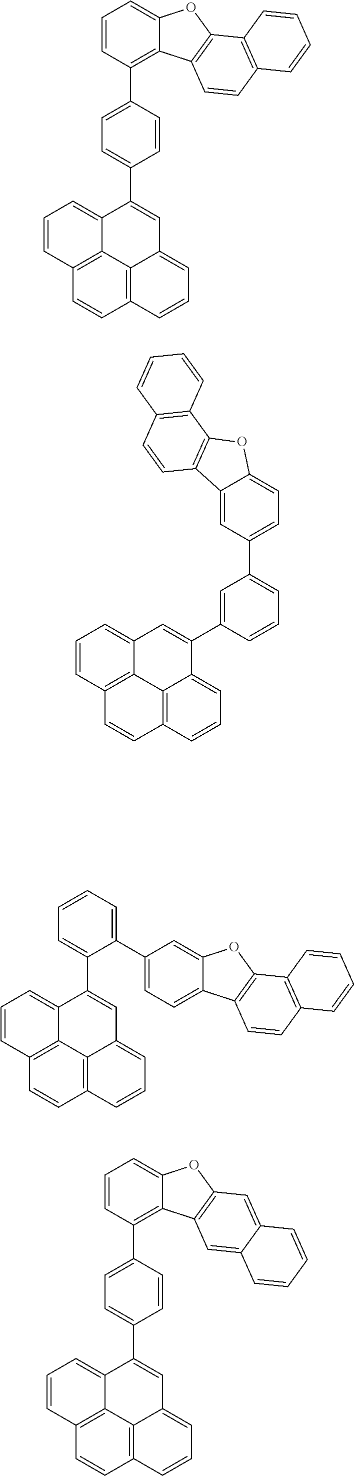

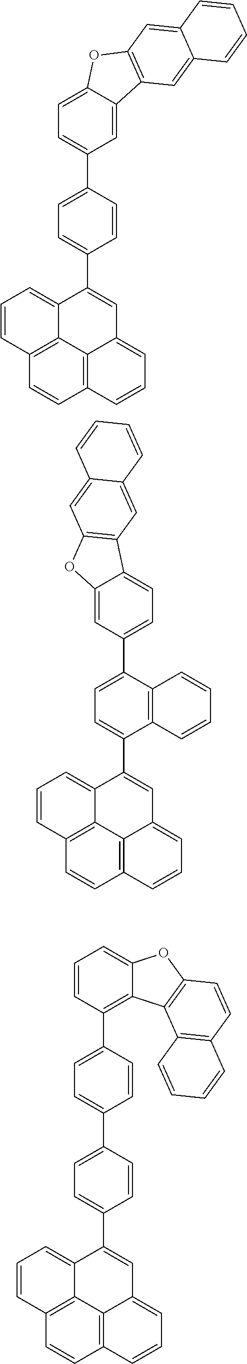

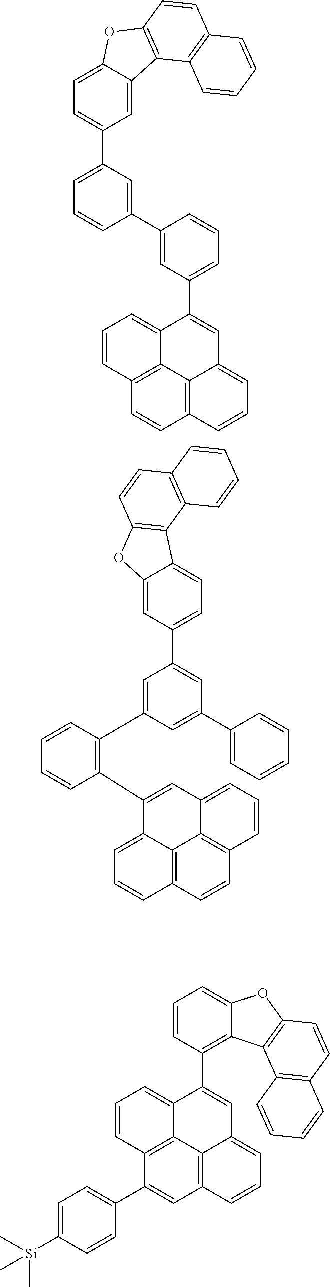

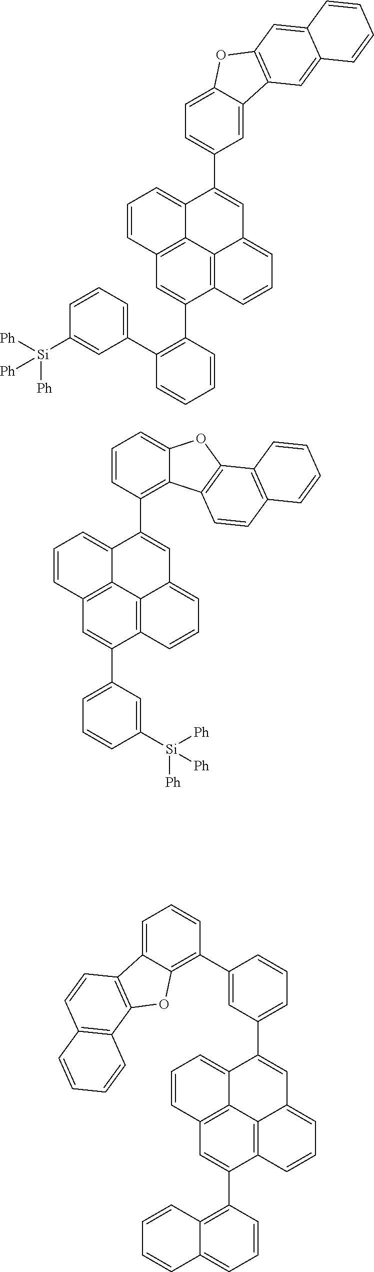

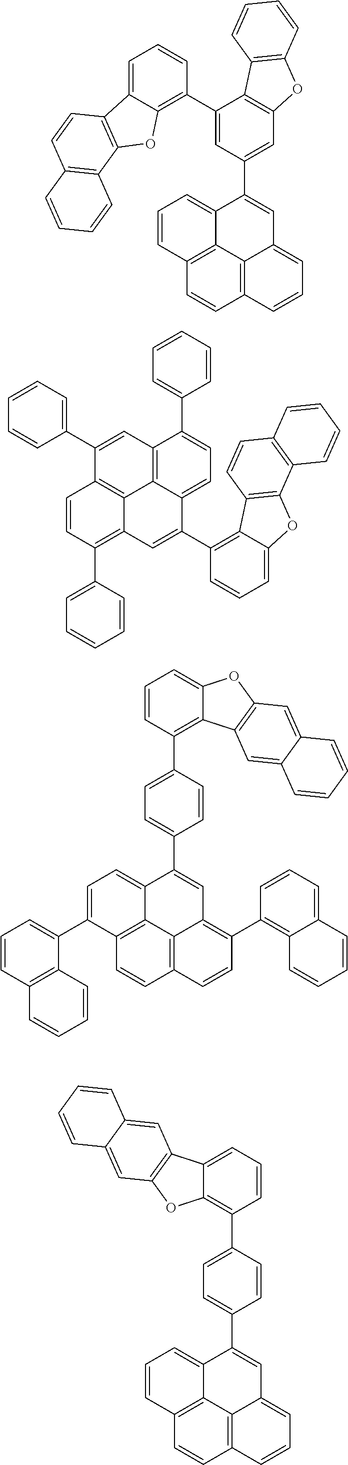

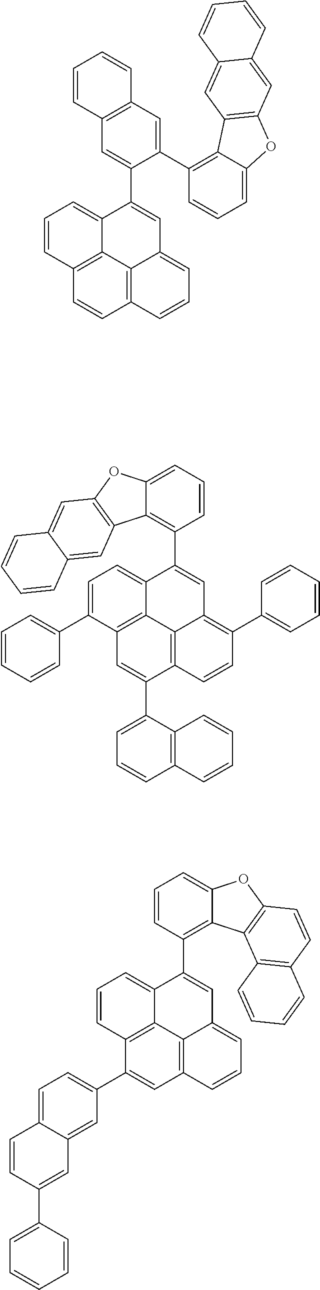 [SMILES: c1ccc2cc(-c3cccc4oc5cc6ccccc6cc5c34)c(-c3cc4cccc5ccc6cccc3c6c54)cc2c1 ; c1ccc(-c2ccc3c(-c4cccc5ccccc45)cc4c(-c5ccccc5)ccc5c(-c6cccc7oc8cc9ccccc9cc8c67)cc2c3c45)cc1 ; c1ccc(-c2ccc3ccc(-c4cc5cccc6cc(-c7cccc8oc9ccc%10ccccc%10c9c78)c7cccc4c7c56)cc3c2)cc1]

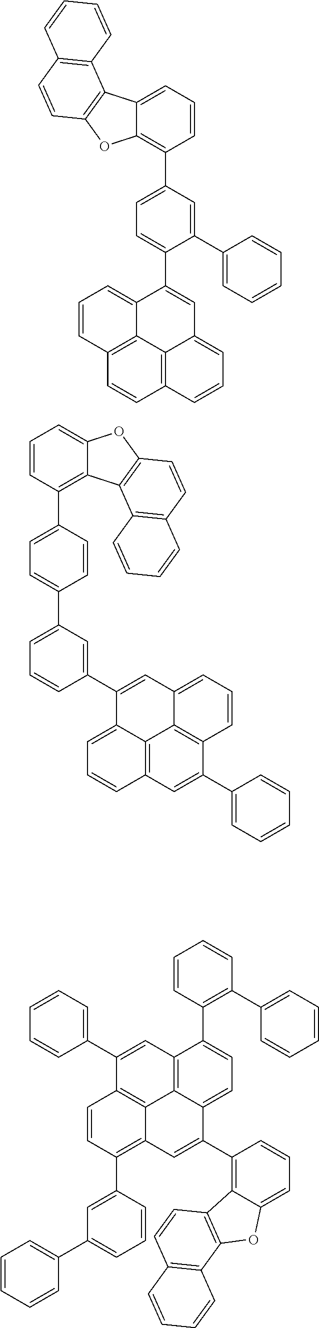

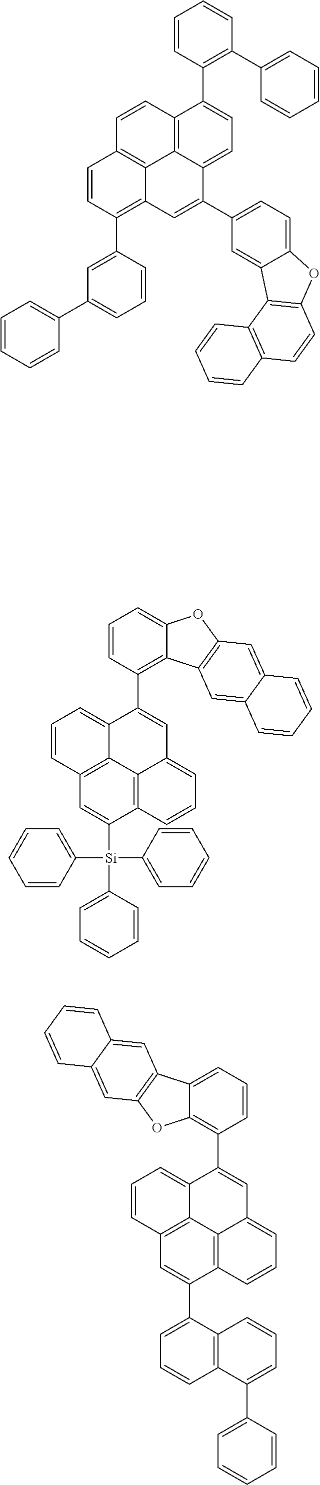 [SMILES: c1ccc(-c2cccc(-c3ccc4ccc5c(-c6ccccc6-c6ccccc6)ccc6c(-c7ccc8oc9ccc%10ccccc%10c9c8c7)cc3c4c65)c2)cc1 ; c1ccc([Si](c2ccccc2)(c2ccccc2)c2cc3cccc4c(-c5cccc6oc7cc8ccccc8cc7c56)cc5cccc2c5c34)cc1 ; c1ccc(-c2cccc3c(-c4cc5cccc6c(-c7cccc8c7oc7cc9ccccc9cc78)cc7cccc4c7c56)cccc23)cc1]

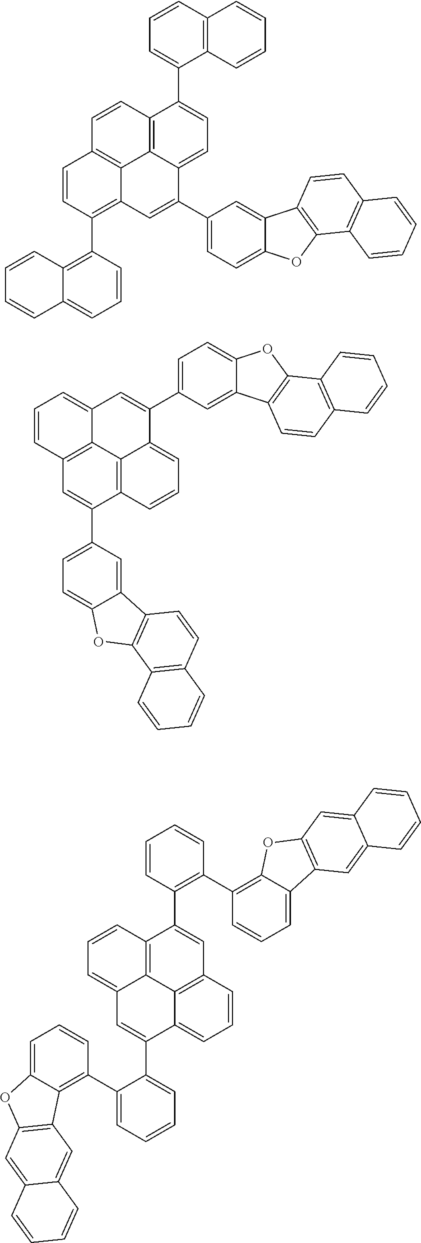 [SMILES: c1ccc2c(-c3ccc4ccc5c(-c6cccc7ccccc67)ccc6c(-c7ccc8oc9c%10ccccc%10ccc9c8c7)cc3c4c65)cccc2c1 ; c1ccc2c(c1)ccc1c3cc(-c4cc5cccc6cc(-c7ccc8oc9c%10ccccc%10ccc9c8c7)c7cccc4c7c56)ccc3oc21 ; c1ccc(-c2cccc3c2oc2cc4ccccc4cc23)c(-c2cc3cccc4c(-c5ccccc5-c5cccc6oc7cc8ccccc8cc7c56)cc5cccc2c5c34)c1]

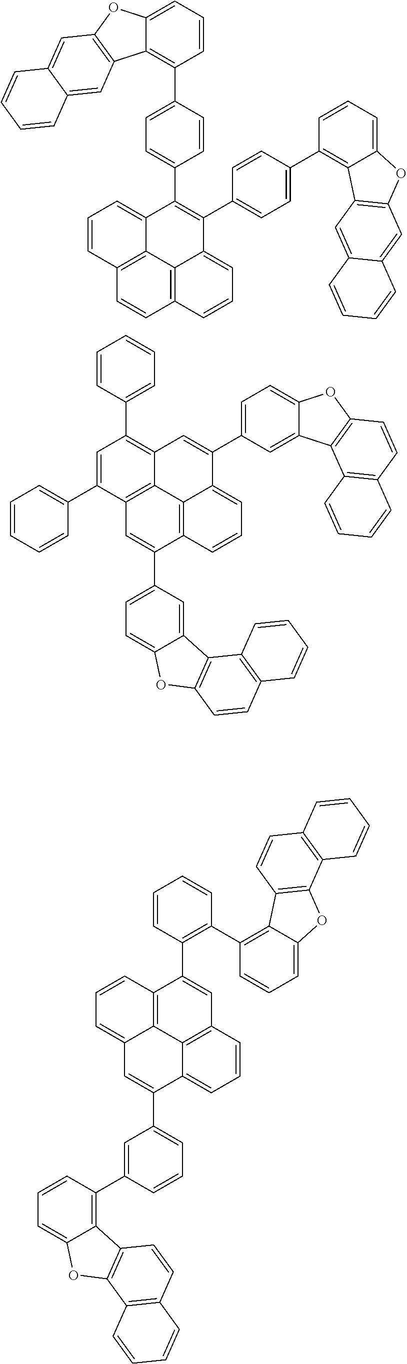

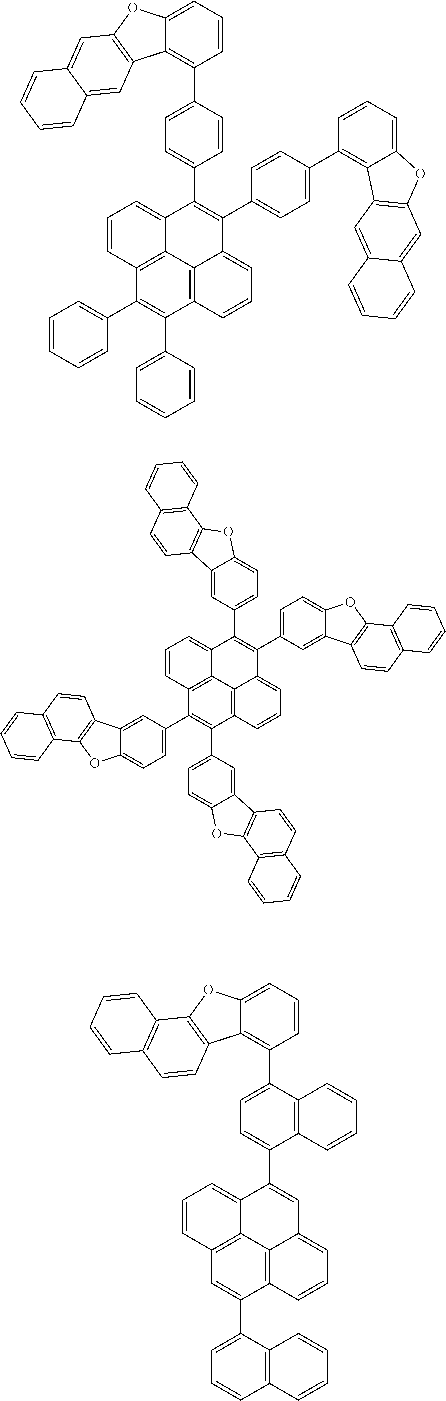 [SMILES: c1ccc(-c2c(-c3ccccc3)c3cccc4c(-c5ccc(-c6cccc7oc8cc9ccccc9cc8c67)cc5)c(-c5ccc(-c6cccc7oc8cc9ccccc9cc8c67)cc5)c5cccc2c5c34)cc1 ; c1ccc2c(c1)ccc1c3cc(-c4c(-c5ccc6oc7c8ccccc8ccc7c6c5)c5cccc6c(-c7ccc8oc9c%10ccccc%10ccc9c8c7)c(-c7ccc8oc9c%10ccccc%10ccc9c8c7)c7cccc4c7c56)ccc3oc21 ; c1ccc2c(-c3cc4cccc5c(-c6ccc(-c7cccc8oc9c%10ccccc%10ccc9c78)c7ccccc67)cc6cccc3c6c45)cccc2c1]

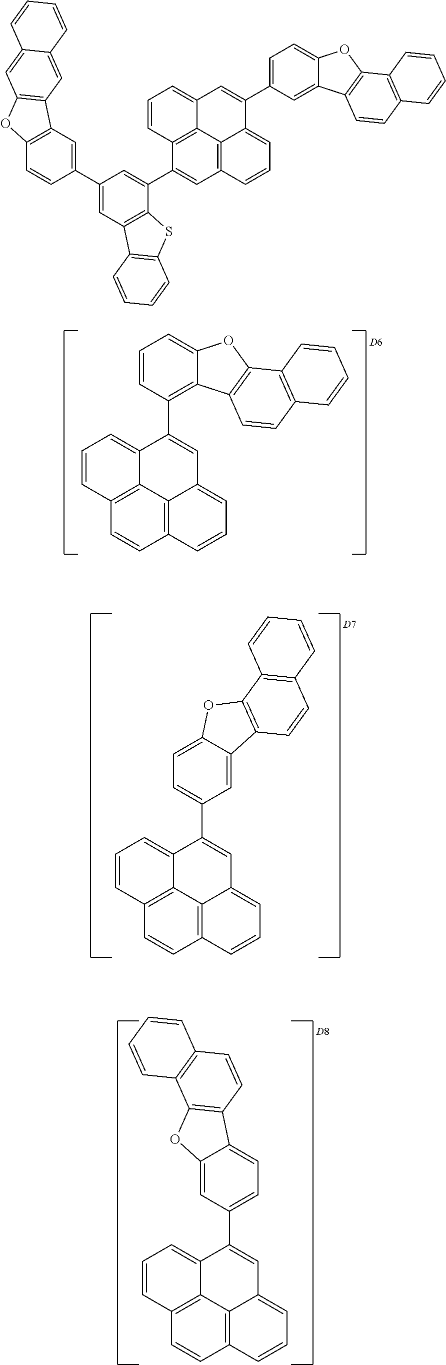

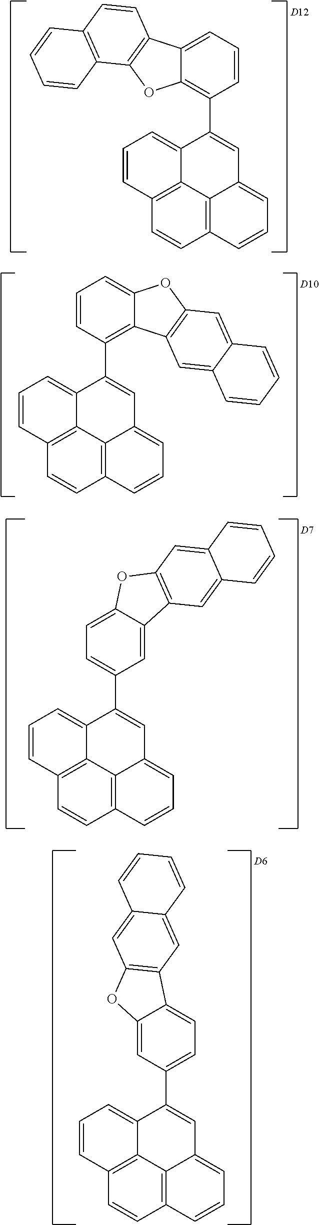

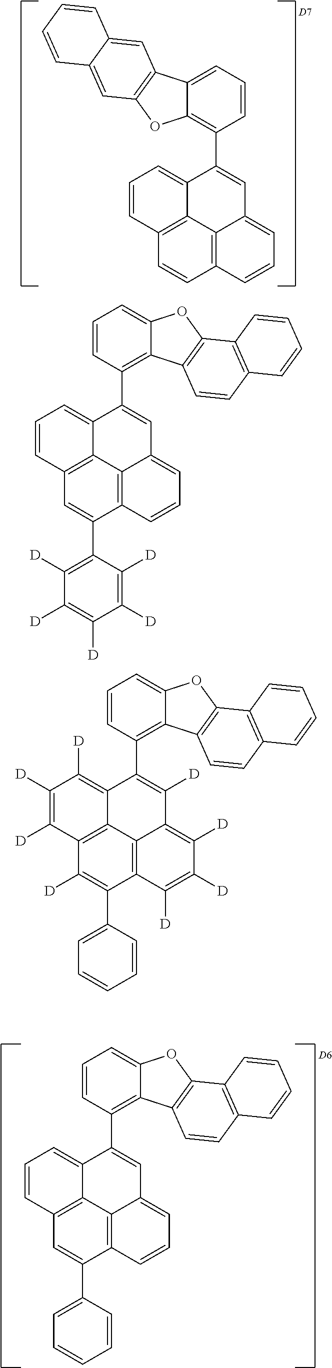

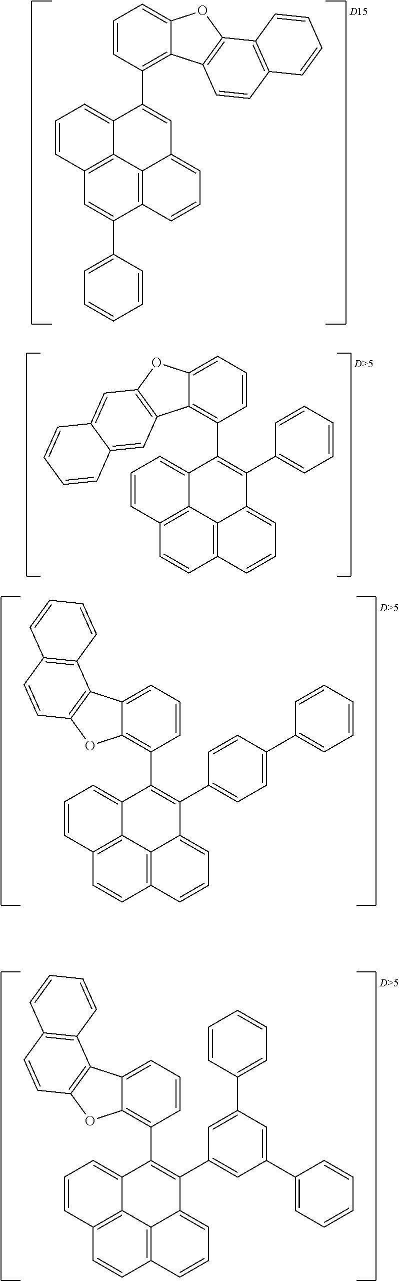

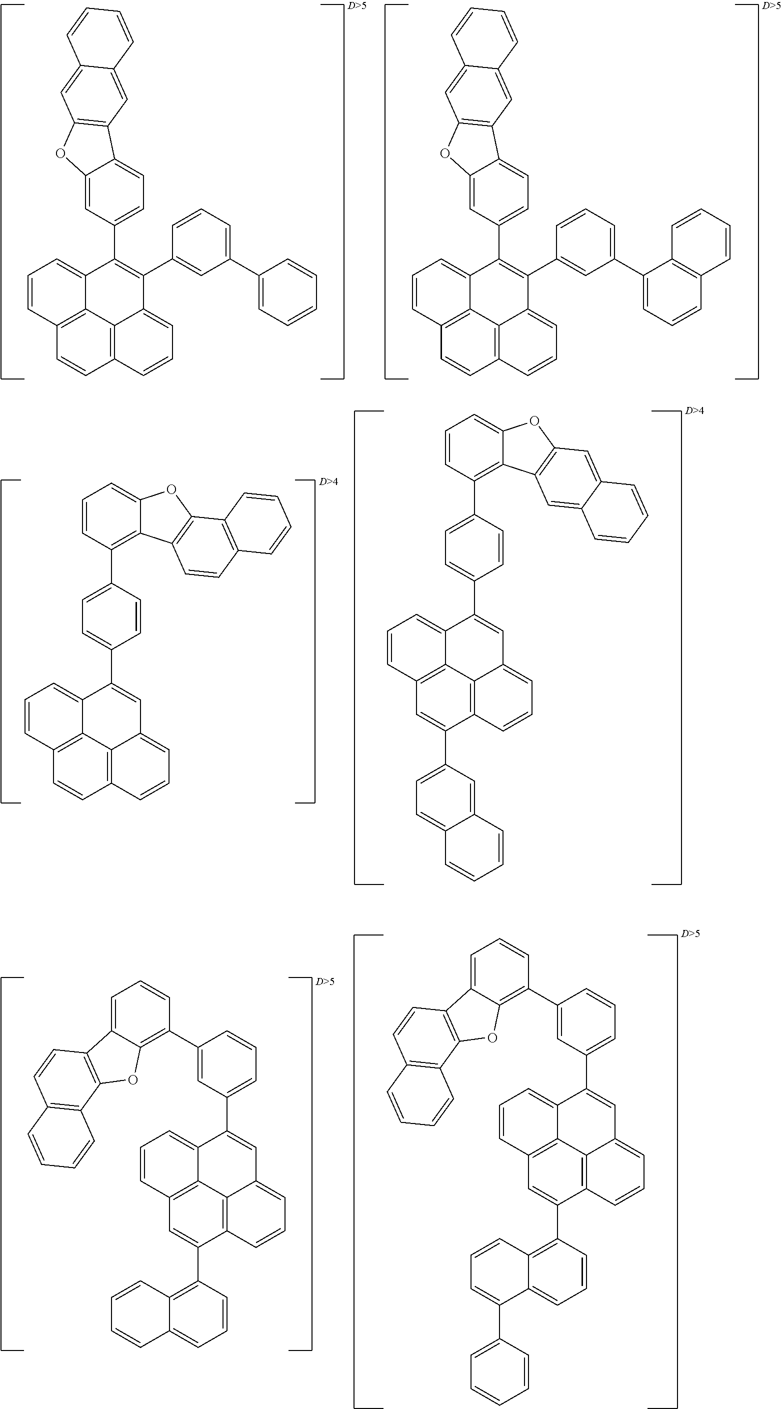

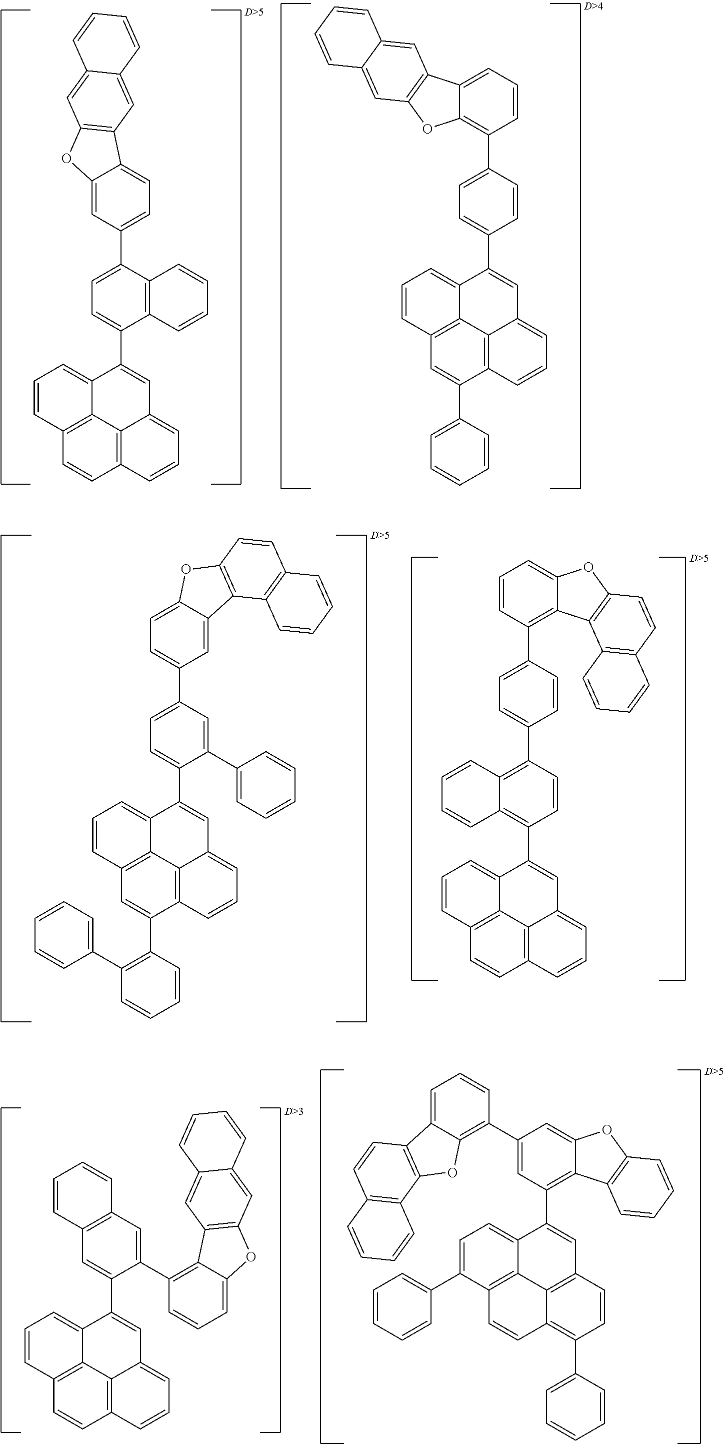

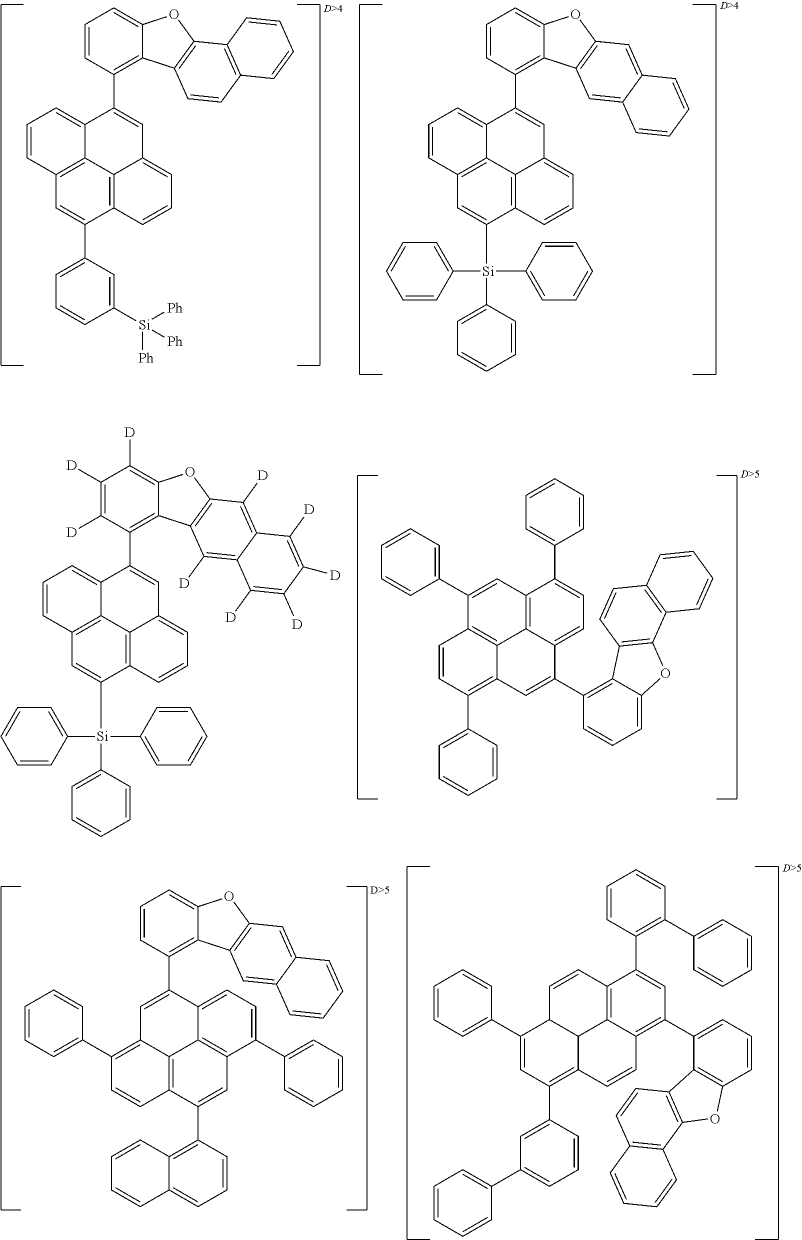

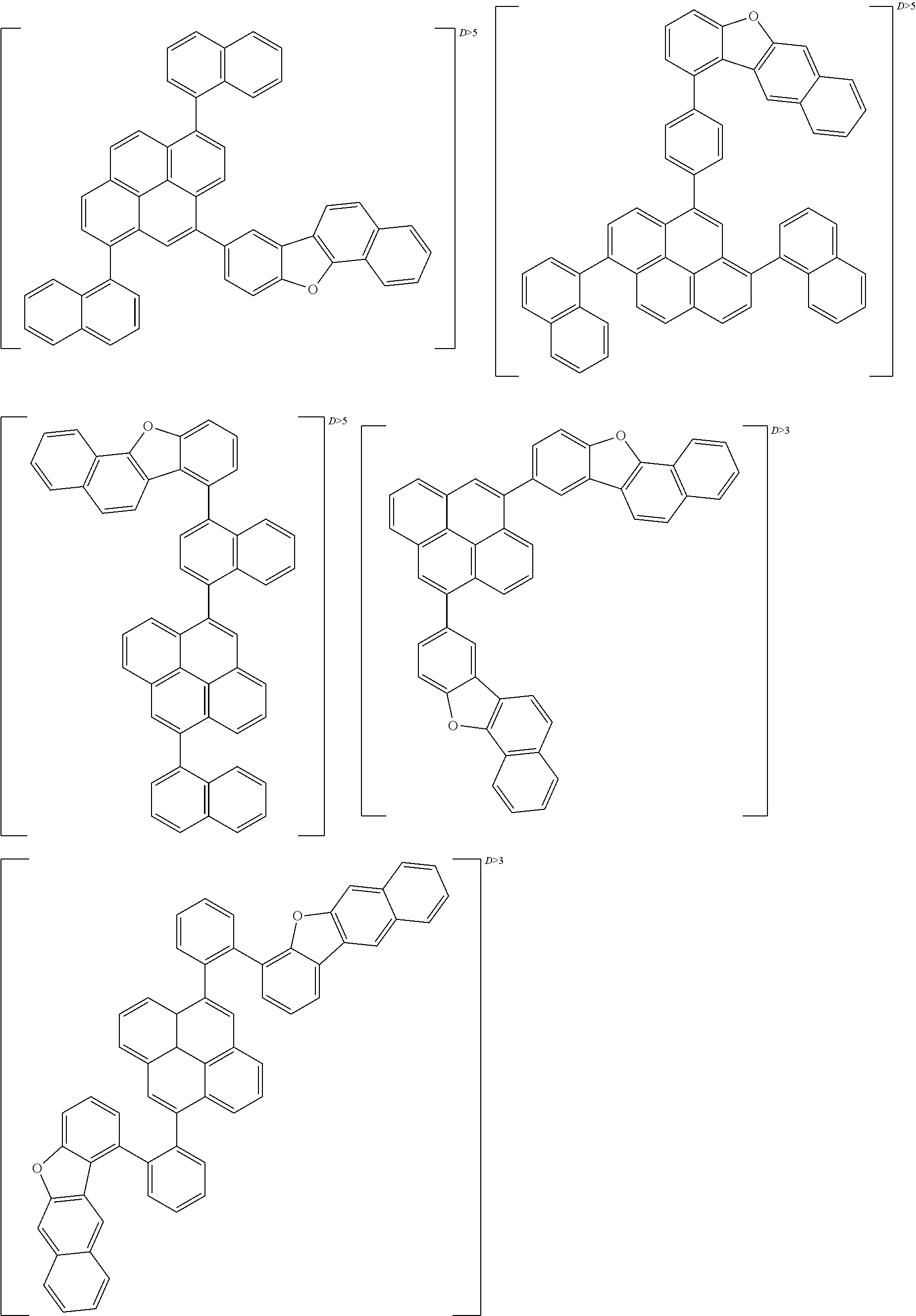

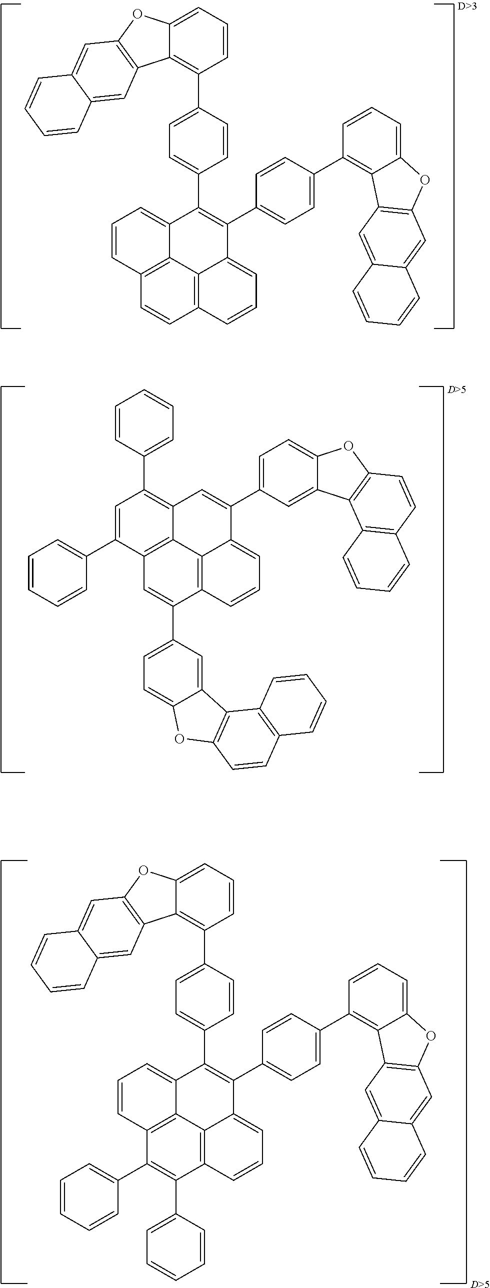

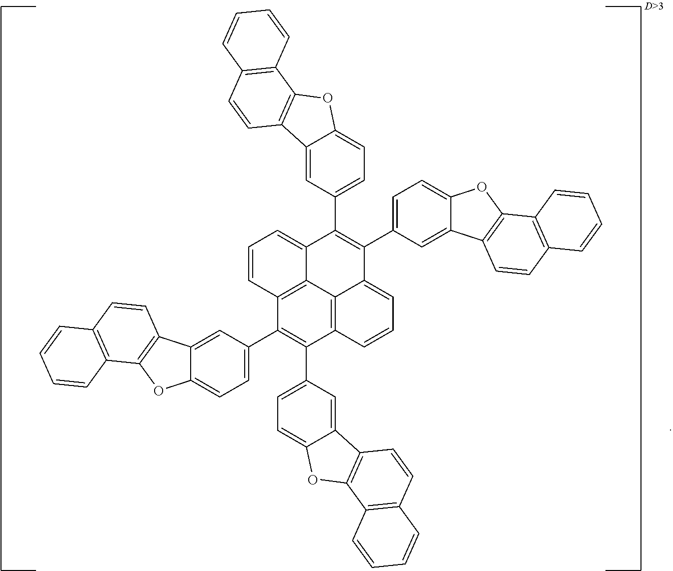

8. An organic light-emitting device comprising an anode; a cathode; and one or more organic layer provided between the anode and the cathode, wherein one or more of the organic layer comprises the compound according to claim 1.

9. The organic light-emitting device according to claim 8, wherein the organic layer comprises a light-emitting layer, and the light-emitting layer comprises the compound.

10. The organic light-emitting device according to claim 9, wherein the light-emitting layer comprises the compound as a host.

11. The organic light-emitting device according to claim 9, wherein the light-emitting layer comprises the compound as a host and further comprises a dopant.

12. The organic light-emitting device according to claim 11, wherein the dopant is a compound of Chemical Formula D or E: 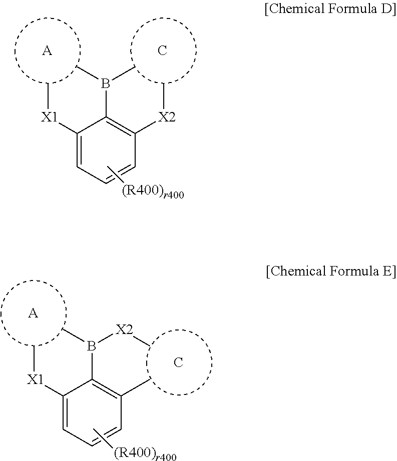
in Chemical Formula D and E,
X1 and X2, which are identical or different, are independently CR′; or NR″; and at least one of X1 and X2 is NR″;
A and C, which are identical or different, are independently a substituted or unsubstituted hydrocarbon ring; or a substituted or unsubstituted heterocycle,
R400, R′ and R″, which are identical or different, are independently hydrogen; deuterium; a halogen group; a nitrile group; a substituted or unsubstituted alkyl group; a substituted or unsubstituted alkenyl group; a substituted or unsubstituted cycloalkyl group; a substituted or unsubstituted alkoxy group; a substituted or unsubstituted alkylthioxy group; a substituted or unsubstituted aryloxy group; a substituted or unsubstituted arylthioxy group; a substituted or unsubstituted silyl group; a substituted or unsubstituted amine group; a substituted or unsubstituted aryl group; or a substituted or unsubstituted heterocyclic group, or it is optionally bonded to an adjacent group to form a substituted or unsubstituted ring, and
r400 is an integer from 0 to 3, and the substituents in the parentheses are identical or different if r400 is 2 or greater.

13. The organic light-emitting device according to claim 12, wherein Chemical Formula D is Chemical Formula D-1 or Chemical Formula D-2: 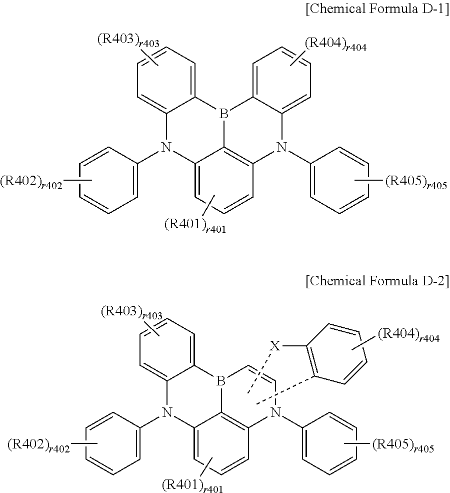
in Chemical Formulae D-1 and D-2,
X is O or S,
R401 to R405, which are identical or different, are independently hydrogen; deuterium; a halogen group; a nitrile group; a substituted or unsubstituted alkyl group; a substituted or unsubstituted alkenyl group; a substituted or unsubstituted cycloalkyl group; a substituted or unsubstituted alkoxy group; a substituted or unsubstituted alkylthioxy group; a substituted or unsubstituted aryloxy group; a substituted or unsubstituted arylthioxy group; a substituted or unsubstituted silyl group; a substituted or unsubstituted amine group; a substituted or unsubstituted aryl group; or a substituted or unsubstituted heterocyclic group, or it is optionally bonded to an adjacent group to form a substituted or unsubstituted cycloalkane ring; a substituted or unsubstituted aromatic hydrocarbon ring; or a substituted or unsubstituted heterocycle,
each of r401 to r405 is an integer from 0 to 3,
two or more R401s are identical or different if r401 is 2 or greater,
two or more R402s are identical or different if r402 is 2 or greater,
two or more R403s are identical or different if r403 is 2 or greater,
two or more R404s are identical or different if r404 is 2 or greater, and
two or more R405s are identical or different if r405 is 2 or greater.

14. The organic light-emitting device according to claim 12, wherein Chemical Formula E is Chemical Formula E-1: 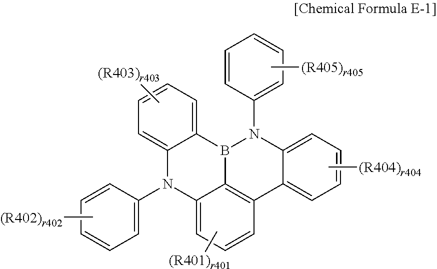
in Chemical Formula E-1,
R401 to R405, which are identical or different, are independently hydrogen; deuterium; a halogen group; a nitrile group; a substituted or unsubstituted alkyl group; a substituted or unsubstituted alkenyl group; a substituted or unsubstituted cycloalkyl group; a substituted or unsubstituted alkoxy group; a substituted or unsubstituted alkylthioxy group; a substituted or unsubstituted aryloxy group; a substituted or unsubstituted arylthioxy group; a substituted or unsubstituted silyl group; a substituted or unsubstituted amine group; a substituted or unsubstituted aryl group; or a substituted or unsubstituted heterocyclic group, or it is optionally bonded to an adjacent group to form a substituted or unsubstituted cycloalkane ring; a substituted or unsubstituted aromatic hydrocarbon ring; or a substituted or unsubstituted heterocycle,
each of r401 to r405 is an integer from 0 to 3,
two or more R401s are identical or different if r401 is 2 or greater,
two or more R402s are identical or different if r402 is 2 or greater,
two or more R403s are identical or different if r403 is 2 or greater,
two or more R404s are identical or different if r404 is 2 or greater, and
two or more R405s are identical or different if r405 is 2 or greater.

15. The organic light-emitting device according to claim 9, wherein
the light-emitting layer comprises the compound of Chemical Formula 1 as a second host and further comprises a first host, and
the first host is a compound of Chemical Formula H: 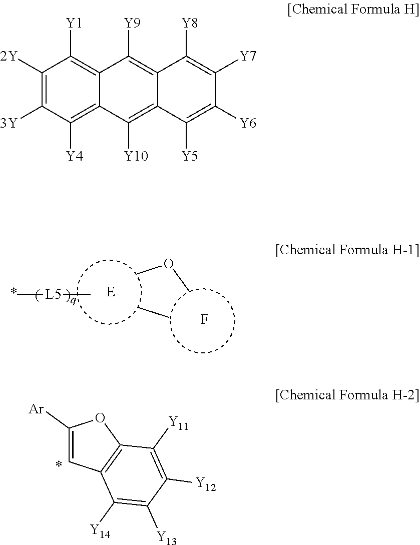
in Chemical Formula H, H-1 and H-2,
one or more of Y1 to Y10 is bonded to the * site of Chemical Formulas H-1 or H-2, and the remainder of Y1 to Y10 not bonded to Chemical Formula H-1 or H-2, and Y11 to Y14, which are identical or different, are independently hydrogen, deuterium, a substituted or unsubstituted aryl group, a substituted or unsubstituted cycloalkyl group, a substituted or unsubstituted heteroaryl group, or a substituted or unsubstituted silyl group,
E and F, which are identical or different, are independently a substituted or unsubstituted aromatic hydrocarbon ring; or a substituted or unsubstituted aromatic heterocycle,
L5 is a direct bond, a substituted or unsubstituted arylene group, or a substituted or unsubstituted heteroarylene group,
q is an integer from 1 to 3, and
Ar is a substituted or unsubstituted aryl group, or a substituted or unsubstituted heteroaryl group.

16. The organic light-emitting device according to claim 9, wherein
the light-emitting layer comprises a first light-emitting layer provided between the anode and the cathode; and a second light-emitting layer provided between the first light-emitting layer and the cathode and in contact with the first light-emitting layer,
the first light-emitting layer comprises the compound of Chemical Formula 1, and
the second light-emitting layer comprises a compound of Chemical Formula H: 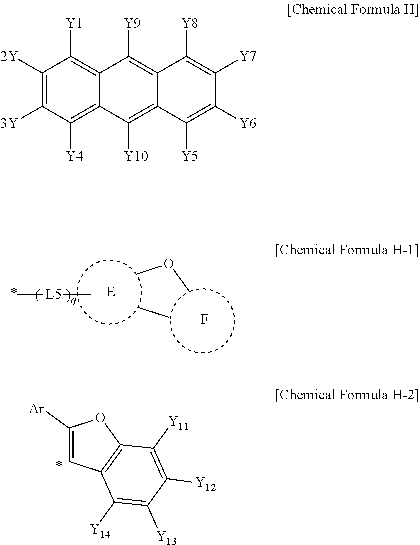
in Chemical Formula H, H-1 and H-2,
one or more of Y1 to Y10 is bonded to the * site of Chemical Formulas H-1 or H-2, and the remainder of Y1 to Y10 not bonded to Chemical Formula H-1 or H-2, and Y11 to Y14, which are identical or different, are independently hydrogen, deuterium, a substituted or unsubstituted aryl group, a substituted or unsubstituted cycloalkyl group, a substituted or unsubstituted heteroaryl group, or a substituted or unsubstituted silyl group,
E and F, which are identical or different, are independently a substituted or unsubstituted aromatic hydrocarbon ring; or a substituted or unsubstituted aromatic heterocycle,
L5 is a direct bond, a substituted or unsubstituted arylene group, or a substituted or unsubstituted heteroarylene group,
q is an integer from 1 to 3, and
Ar is a substituted or unsubstituted aryl group, or a substituted or unsubstituted heteroaryl group.

17. The organic light-emitting device according to claim 16, wherein
the light-emitting layer further comprises a third light-emitting layer provided between the second light-emitting layer and the cathode and in contact with the second light-emitting layer, and
the third light-emitting layer comprises the compound of Chemical Formula 1.
